(19) United States
(12) Patent Application Publication
(10) Pub. No.: US 2025/0312320 A1
(43) Pub. Date: Oct. 9, 2025

(54) METHOD AND TREATMENT FOR OSTEOARTHRITIS AND DISEASES OF CHONDROCYTE HYPERTROPHY

(71) Applicant: The Royal Veterinary College, London (GB)

(72) Inventors: Scott Roberts, London (GB); Leah Wells, London (GB)

(73) Assignee: The Royal Veterinary College, London (GB)

(21) Appl. No.: 18/872,124

(22) PCT Filed: Jun. 5, 2023
(86) PCT No.: PCT/GB2023/051465

§ 371 Date: Dec. 5, 2024

(30) Foreign Application Priority Data
Jun. 6, 2022 (GB) 2208226.7

(51) Int. Cl. A61K 31/4353; A61K 31/44; A61K 31/47; A61K 31/496; A61P 19/02; A61P 19/04; A61P 19/08; C40B 30/06; G01N 33/50

(52) U.S. Cl. CPC A61K 31/4353; A61K 31/44; A61K 31/47; A61K 31/496; A61P 19/02; A61P 19/04; A61P 19/08; C40B 30/06; G01N 33/5008; G01N 33/502; G01N 2400/10

(57) ABSTRACT

The disclosure concerns a method of treating a disorder characterised by chondrocyte hypertrophy e.g. osteoarthritis, and a composition for use in such a method. The disclosure also concerns a method of producing a miniaturised model of endochondral ossification, and a miniaturised model of endochondral ossification producible by such method. The disclosure further provides a method of screening for compositions for use in treating a disorder characterised by chondrocyte hypertrophy, and a composition identified by such method.

### FIELD OF THE DISCLOSURE

The disclosure concerns a method of treating a disorder characterised by chondrocyte hypertrophy such as osteoarthritis, and a composition for use in such a method. The disclosure also concerns a method of producing a miniaturised model of endochondral ossification, and a miniaturised model of endochondral ossification producible by such method. The disclosure further provides a method of screening for compositions for use in treating a disorder characterised by chondrocyte hypertrophy, and a composition identified by such method.

### BACKGROUND

Osteoarthritis is the most common form of arthritis, and a major cause of joint pain and disability. The prevalence of osteoarthritis is steadily increasing, and it is expected that osteoarthritis will ultimately be the single greatest cause of disability in the general population. It is estimated that by 2050, 130 million people worldwide will suffer from osteoarthritis, with 40 million will being severely disabled by the disease. Recent statistics (2014) for England indicate that 52% of the over 50s report osteoarthritis in at least one of four joint regions (hand, hip, foot, knee), with about 22% reporting disabling osteoarthritis.

Osteoarthritis is a degenerative joint disease that results from the breakdown of joint cartilage and underlying bone. Osteoarthritis is, though, a “whole joint disease”, involving changes in many joint-associated tissues. Such changes may include loss of articular cartilage, exposure of underlying bone, synovial inflammation, narrowing of the joint, thickening of the joint capsule, development of osteophytes, subchondral sclerosis, development of bone cysts, and degeneration of menisci. The primary symptoms resulting from such changes are joint pain and stiffness.

Osteoarthritis is thought to arise (at least partially) from a disorder in chondrogenic differentiation (FIG. 1). Chondrogenic differentiation is an important part of endochondral ossification, the process by which cartilage forms bone during skeletal development, bones grow longitudinally during growth postnatally and bones repair following fracture. Chondrocytes are resident cartilage cells which are ultimately derived from mesenchymal cells. During endochondral ossification, chondrocytes follow a differentiation pathway to form a cartilaginous tissue, and then further differentiate towards hypertrophic chondrocytes. Mineralisation of the matrix by hypertrophic chondrocytes is followed by transdifferentiation of hypertrophic chondrocytes to osteoblasts, which results in the formation of new bone, or by apoptosis.

In healthy articular cartilage, chondrocytes are maintained in a steady state and do not differentiate into hypertrophic chondrocytes permissive of matrix mineralization/bone formation. In this way, cartilage homeostasis is maintained and healthy joint function enabled. In osteoarthritis, this homeostasis is disrupted. Rather than being maintained in a stable, mature form, chondrocytes follow the same differentiation pathway as in endochondral ossification, leading to the generation of hypertrophic chondrocytes. In turn, hypertrophic chondrocytes promote mineralisation of joint cartilage. As a result, cartilage is degraded, with little regeneration. Accordingly, articular cartilage is progressively lost.

Hypertrophic chondrocyte differentiation underlies several other diseases in addition to osteoarthritis. For instance, degenerative disc disease, heterotopic ossification, and hereditary multiple exostoses are all characterised by chondrocyte hypertrophy. In degenerative disc disease, chondrocyte hypertrophy contributes to loss of disc flexibility and subsequent narrowing of the gap between adjacent vertebrae. The function of the intervertebral joint is thereby impaired, leading to pain and inflammation. In heterotopic ossification, chondrocyte hypertrophy contributes the formation of bone tissue outside of the skeleton, in muscle and soft tissues. This can lead to firm swellings that may be tender to the touch and reduce the range of motion of the joint served by the muscle or soft tissue. In hereditary multiple exostoses, chondrocyte hypertrophy contributes to the development of benign osteocartilaginous masses towards the ends of long bones of the limbs or on flat bones such as the pelvic bone or scapula. These masses are commonly known as exostoses or osteochondromas, and can disrupt physeal growth, limit the range of motion, and cause joint pain. Conversely, atrophic non-union following bone fracture is characterised by an absence of chondrocyte hypertrophy resulting in the permanent failure of fracture repair.

Currently, treatment for osteoarthritis and other disorders characterised by hypertrophic chondrocyte differentiation aim to relieve symptoms and delay disease progression. For example, anti-inflammatories may be administered to manage inflammation, and analgesic drugs may be administered to manage pain. Physical therapy may also assist in these respects. In osteoarthritis, intra-articular injections of viscosity agents and/or steroids may help to relieve symptoms of osteoarthritis. In degenerative disc disease, steroid injections may be administered epidurally. However, disorders characterised by hypertrophic chondrocyte differentiation tend to progress despite these therapies, and surgery may be indicated. For instance, late-stage osteoarthritis may require total joint replacement. Abnormal bone growths (e.g. in heterotopic ossification or hereditary multiple exostoses) may be removed. In degenerative disc disease, it may be necessary to remove all or part of the disc, to fuse vertebrae together, and/or to modify the anatomy of vertebrae to relieve pressure on the spinal cord or nerves.

There is therefore a need for improved treatments for disorders characterised by chondrocyte hypertrophy. There is a particular need for treatments that minimise or stop disease progression, for instance by slowing the loss of healthy cartilage or the inappropriate formation of bone. Treatments that reverse pathogenic changes, for example by promoting the formation of articular or annular or endplate cartilage, are especially desired.

### SUMMARY OF THE DISCLOSURE

The inventors have developed a miniaturised model of endochondral ossification, in which chondrocytes undergo hypertrophic differentiation. The miniaturised model therefore also models the disease process underlying disorders characterised by chondrocyte hypertrophy, such as osteoarthritis, degenerative disc disease, heterotopic ossification and hereditary multiple exostoses. Accordingly, the model can be used to screen for compositions that can be used to treat disorders characterised by chondrocyte hypertrophy, such as agents that minimise or stop disease progression and/or promote cartilage repair. The model can also be used to screen for compositions that can be used to treat a fracture, such as agents that promote hypertrophy, mineralisation and/or bone repair. As the model is miniaturised, it is well-suited to high-throughput screening of agents. By using the model to screen for agents, the inventors have identified that Mediator kinase inhibitors may surprisingly be used to treat disorders characterised by chondrocyte hypertrophy.

Accordingly, the disclosure provides a method of treating a disorder characterised by chondrocyte hypertrophy in an individual, comprising administering a Mediator kinase inhibitor to the individual. The disclosure further provides:
a Mediator kinase inhibitor for use in a method of treating a disorder characterised by chondrocyte hypertrophy in an individual, the method comprising administering the Mediator kinase inhibitor to the individual;
a method of producing a miniaturised model of endochondral ossification, comprising: (a) providing a micromass of ATDC5 cells having a volume of about 1 μl to about 7 μl; (b) culturing the micromass in differentiation medium for at least 7 days; and (c) culturing the micromass in mineralisation medium for a further 7 days;
a miniaturised model of endochondral ossification, producible by the method of the disclosure;
a method of screening for compositions for use in treating (1) a disorder characterised by chondrocyte hypertrophy or (2) a fracture, comprising: (a) providing a micromass of ATDC5 cells having a volume of about 1 μl to about 7 μl; (b) culturing the micromass in differentiation medium for at least 7 days, wherein a test composition is provided to the micromass on day 1; (c) culturing the micromass in mineralisation medium comprising the test composition for up to a further 7 days; and (d) quantifying the amount of glycosaminoglycans and/or the degree of mineralisation in the micromass following the culture of step (c); and
a composition for use in treating (1) a disorder characterised by chondrocyte hypertrophy or (2) a fracture, identified by the method of the disclosure.

### DESCRIPTION OF THE FIGURES

FIG. 1: Schematic of chondrogenic differentiation and disorders therein. Chondrocytes are resident cells within cartilage. In growth cartilage during endochondral ossification, chondrocytes follow a distinct differentiation pathway to form a cartilaginous intermediate that paves the way for new bone formation through chondrocyte hypertrophy and cartilage mineralisation. In healthy adult articular cartilage, however, chondrocytes are maintained in a steady state and do not continue differentiation into hypertrophy. Cartilage homeostasis is thus maintained, enabling healthy joint function. In osteoarthritis, chondrocytes are pushed into hypertrophy following the same differentiation pathway seen in endochondral ossification (although initiators may be different), leading to an imbalance whereby cartilage degradation is apparent with little regeneration. Articular cartilage is thus progressively lost.

FIG. 2: Morphological Differences in BI1347 Treated ATDC5 Micromasses. ATDC5 Micromasses treated with 1 μM BI1347 for 14 days display a visible increase in extracellular matrix production with micromasses observably larger, rounder and have a glossy appearance closely resembling cartilage.

FIG. 3: Schematic outlining the optimised screening platform methodology. Chondrogenic cells are seeded onto 96-well plates at high densities of 5.4×10+ cells per high density culture (micromass). Upon addition of differentiation media, cells are co-treated with library at desired concentration for 7 days before induction of mineralisation for a following 7 days. Micromass plates can then be fixed and stained for observation of effects of library on chondrogenesis. In the case of screening of endochondral ossification effectors on chondrocyte hypertrophy, this may be observing a reduction in mineralisation.

FIG. 4: Absorbance Ratio Identifying BI1347 In The Epigenetic Library Screen. (A) Alcian blue:alizarin red ratios for each compound identified BI1347 as a modulator of endochondral ossification, that maintains/promotes healthy differentiation and impedes hypertrophic, disease-like differentiation in ATDC5 cell line. Lilac data points represent DMSO control ratios with dotted line indicating the window of DMSO control ratios. (B) Images of alizarin red and alcian blue stained wells from DMSO control, BI1347 and BI1374 negative control prior to stain leaching.

FIG. 5: Scanned Images of 96-well Plate Screening Method. (A) No observed difference in Micromass phenotype when compound library is added at the point of mineralisation after 2 weeks of differentiation. (B) A variety of mass phenotypes observed when compound is added at point of differentiation for the duration of the 2-week screen.

FIG. 6: BI1347 Treatment of ATDC5 and Primary Equine Chondrocytes Gene Expression Analysis. (A) ATDC5 micromass treated with 1 μM BI1347 showed a 6-fold Increase in ACAN (N.S) and 4.7-fold COL2A1 (p≤0.05) expression at day 7 and a 6-fold increase in ACAN (p≤0.05) and 5.3-fold decrease in COL10A1 (p≤0.01) on day 14 of culture when compared to DMSO control. (B) Primary equine chondrocytes (N=1) treated with 1 μM BI1347 showed a 9-fold increase in COL2A1 (p≤0.05), 1.5-fold increase in SOX9 (N.S) and 14-fold increase in ACAN (p≤0.01) expression compared to DMSO control, with identical RUNX2 expression observed in the two groups. Error bars=SD, statistical analysis=T-test with Welch correction and N=3 unless otherwise stated.

FIG. 7: IL-1β Induced Degradation of ATDC5 Micromass BI1347 Treated Gene Expression Analysis. (A) ACAN expression analysis of ATDC5 micromass treated without or with 10 ng/ml IL-1β and 1 μM BI1347 from days 12-14 (1.2-fold (N.S) and 3-fold (p≤0.05) and increase respectively). Addition of 1 μM BI1347 throughout the differentiation period with 10 ng/ml IL-1β treatment on day 12-14 increased expression 20-fold compared to DMSO control (p≤0.01). (B) COL2A1 expression analysis of ATDC5 without or with 10 ng/ml IL-1β and 1 μM BI1347 from days 12-14 (1.7-fold (p≤0.01) and 1.5-fold (p≤0.05) increase respectively). Addition of 1 μM BI1347 throughout the differentiation period with 10 ng/ml IL-1β from days 12-14 increased expression 3-fold compared to DMSO control (p≤0.01). (Error bars=SD, statistical analysis=T-test with Welch correction and N=3 unless otherwise stated.)

FIG. 8: Dose Response of CDK8 inhibitors on ATDC5 Micromass. Similar trends observed in effects on alizarin red and alcian blue staining across CDK8 inhibitors with a progressive decrease in alizarin red (black) with increasing concentrations of CDK8 inhibitors whilst alcian blue staining (purple) is generally maintained. Data plotted using log (inhibitor) vs. response-Variable slope fit (four parameters).

FIG. 9: Dose Response of CDK8 Inhibitors on ATDC5 Micromass Stained Plate Images. A decrease in alizarin red (left) and maintenance of alcian blue (right) staining observed with increasing CDK8 inhibition across compounds, with micromasses having a similar appearance.

FIG. 10: Primary Osteoclast Treatment with BI1347. C57/BL-6 bone marrow cells treated with 1 μM BI1347 show no change in pre-osteoclast or mature osteoclast number compared to DMSO control, however, a 2.3-fold reduction in osteoclast resorptive activity is observed (p≤0.0001). (Error bars=SD, statistical analysis=T-test with Welch correction and N=8)

FIG. 11: Osteoblast-like cell Treatment with BI1347. (A) MC3T3 treated with 1 μM BI1347 display reduced mineralisation by alizarin red stain over 14 days culture compared to DMSO control. Imaged wells are from day 14 stained cultures. (B) Cell viability assay via resazurin staining indicates no toxicity observed in MC3T3 cells treated with 1 μM BI1347. C) Microscopy of MC3T3 cells differentiated with 50 μg/ml ascorbate acid 2 phosphate (AA, day 0-7) then 50 μg/ml AA+2 mM βGlycerophosphate (BGP, 7-14). Fresh AA, BGP and 100 nM MSC or DMSO added each media change, note ablation of mineralization in MSC treated cultures. D) Quantification of alizarin red dye (mineralization) leached from culture after 14 days (n=3, **p=≤0.01). (Error bars=SD, statistical analysis=T-test with Welch correction and N=3)

FIG. 12: Macrophage Treatment with BI1347. THP-1 derived Macrophages treated with 1 μM BI1347 display significantly decreased gene expression of the pro-inflammatory mediator TLR4 (9-fold p≤0.01) and NF-κB (6-fold p≤0.05) and an upregulated gene expression of anti-inflammatory mediator PPAR-Y (11-fold p≤0.01) compared to DMSO control. (Error bars=SD, statistical analysis=T-test with Welch correction and N=3 unless otherwise stated.)

FIG. 13: RNA-Seq Cluster Analysis of Differentially Expressed Genes in BI1347 Treatment Vs DMSO. FPKM cluster analysis, clustered using the log 2 (FPKM+1) value. Wide-ranging differences in high gene expression (Red) and low gene expression (Blue) in BI1347 treated micromasses compared to DMSO control indicating CDK8 inhibition alters cell phenotype.

FIG. 14: Upregulated Biological Processes Observed in ATDC5 Treated with B11347. Collagen fibril organisation (10-fold), Chondrocyte differentiation (6-fold), cartilage development (5.5-fold) and extracellular matrix organisation (5-fold) are all highlighted in the top 10 enriched biological processes in BI1347 treatment.

FIG. 15: Downregulated Biological Processes Observed in ATDC5 Treated With B11347. The top 10 downregulated pathways are mostly immune system related, such as response to interferon-beta (15-fold), innate immune response (3-fold) and response to cytokines (3-fold).

FIG. 16: Dose response with B11347 and MSC2530818 treatment on genes of interest (from RNAseq). ATDC5 micromasses treated with 1 μM, 100 nM or 10 nM of CDK8i (BI1347 or MSC2530818) or DMSO control during 14 day hypertrophic differentiation. All OA protective genes (TIMP4, MATN3, SMOC2, CNMD) display a dose dependent increase in gene expression in response to CDK8i The hypertrophic/mineralised matrix marker IBSP displays a dose dependent decrease. (all data N=3, error bars=SD, all significant from DMSO control p=≤0.05)

FIG. 17: Phased dosing of BI1347 and MSC250818 with ATDC5 micromasses during 14 days differentiation to hypertrophy. A) Glycosaminoglycan (GAG) deposition (leached alcian blue absorbances) from micromasses treated with a phased dosing schedule of 1 μM MSC2530818, BI1347 or DMSO control throughout. Note the increase in GAG deposition from DMSO control at all timepoints (top). Scanned images of alcian blue stained micromasses treated with phased dosing of MSC2530818, B11347 or DMSO prior to leaching (bottom). B) Mineralization (leached alizarin red absorbances) from micromasses treated with a phased dosing schedule of 1 μM MSC2530818, BI1347 or DMSO control throughout. Note the decrease in mineralisation from DMSO control at all timepoints (top). Scanned images of alizarin red stained micromasses treated with a phased dosing schedule of 1 μM MSC2530818, BI1347 or DMSO prior to leaching (bottom). (all data N=3, error bars=SD, *p=≤0.05, **p=≤0.01, ***p=≤0.001. ****p=≤0.0001 significance from DMSO control.) Phased dosing refers to number of days treated with CDK8i prior to switching to control media (i.e. 2 refers to 2 days of CDK8i treatment followed by 12 days of control media treatment).

FIG. 18: Mediator complex gene expression in ATDC5 micromasses treated with BI1347 or DMSO. Gene expression analysis on days 7 and 14 of ATDC5 treated with 1 μM B11347 or DMSO control. Note overall reduction of expression at genes on day 7 with B11347 treatment compared to DMSO control, suggesting early modulation of mediator components *p=≤0.05, **p=≤0.01, ***p=≤0.001)

FIG. 19: Treatment of ihMSC with MSC2530818 (MSC) during chondrogenesis and in a IL1β OA disease degradation model. A) Scanned images of ihMSC micromasses treated with 100 nM MSC2530818 (MSC) or DMSO control in chondrogenic (−IL1β, left) or OA disease model (+IL1β, right), note the increase in glycosaminoglycan (GAG; alcian blue stain) intensity with MSC2530818 (MSC) treatment compared to DMSO control in both conditions. B) Gene expression analysis of ihMSC micromasses treated with 100 nM MSC2530818 (MSC) or DMSO control in chondrogenic (−IL1β) or disease model (+IL1β) . Significant increase in healthy matrix component gene expression in chondrogenic (−IL1β) and OA disease model (+IL1β) conditions with MSC2530818 (MSC) treatment for ACAN (3-fold, 1.6-fold respectively) and MATN3 (4-fold, 3-fold respectively), with a 1.3-fold reduction gene expression of the aggrecanase ADAMTS5 (all data N=3, error bars=SD, *p=≤0.05).

FIG. 20: Treatment of primary human articular chondrocytes with MSC2530818 (MSC) in chondrogenesis and IL1β OA disease degradation model. Gene expression analysis of primary human articular chondrocytes treated with 100 nM MSC2530818 (MSC) or DMSO control in chondrogenic (−IL1β) or OA disease model (+IL1β). Significant increase in healthy matrix component gene expression in chondrogenic (−IL1β) and disease model (+IL1β) conditions with MSC2530818 (MSC) treatment for MATN3 (100-fold, 12-fold respectively) and SMOC2 (6-fold, 7-fold respectively) with a 2.5-fold reduction in gene expression of the aggrecanase ADAMTS5 (p=0.053) in the OA disease model. (all data N=1, error bars=SD, *p=≤0.05)

FIG. 21: Treatment of primary canine articular chondrocytes with MSC2530818 (MSC) in chondrogenesis and IL1β OA disease degradation model. Gene expression analysis of primary canine chondrocytes treated with 100 nM MSC2530818 (MSC) or DMSO control in chondrogenic (−IL1β) or OA disease model (+IL1β). Significant increase in healthy matrix component gene expression in chondrogenic (−IL1β) and OA disease model (+IL1β) conditions with MSC2530818 (MSC) treatment for ACAN (2-fold, 3-fold respectively), COL2A1 (5-fold, 13-fold ns, respectively), TIMP4 (4-fold, 1.1-fold ns, respectively), CNMD (7-fold, 6-fold respectively), SMOC2 (2-fold for both conditions), MATN3 (2 fold, 6-fold respectively) with a 1.4-fold reduction gene expression of the aggrecanase ADAMTS5 (ns) in the disease model. (all data N=1, error bars=SD, *p=≤0.05, **p=≤0.01, ***p=≤0.001, ****p=≤0.0001, ns=nonsignificant)

FIG. 22: Treatment of primary equine articular chondrocytes with MSC2530818 (MSC) in chondrogenesis and IL1β OA disease degradation model. Gene expression analysis of primary equine chondrocytes treated with 100 nM MSC2530818 (MSC) or DMSO control in chondrogenic (−IL1β) or OA disease model (+IL1β). Healthy matrix component gene expression from chondrogenic (−IL1β) and OA disease model (+IL1β) conditions with MSC2530818 (MSC) treatment for ACAN (2-fold ns, 1.8-fold ns increase respectively), COL2A1 (1.25-fold decrease ns, 11-fold increase respectively), TIMP4 (no change, 1.14-fold increase ns respectively), CNMD (41-fold, 70-fold increase respectively), MATN3 (2 fold ns, 2-fold ns respectively), SMOC2 (2.3-fold, 3-fold increase), with a 2.7-fold reduction in gene expression of the aggrecanase ADAMTS5 in the OA disease model. (all data N=2, error bars=SD, *p=≤0.05. **p=≤0.01, ***p=≤0.001, ns=nonsignificant)

FIG. 23: Metabolic analysis of hypertrophic ATDC5 treated with MSC2530818 (MSC) or DMSO control. Basal metabolic analysis of hypertrophic ATDC5 micromasses treated with 1 μM MSC2530818 or DMSO control via the MitoStress Test SeaHorse XF (Agilent). A) Real time measurements of basal oxygen consumption rate (OCR, left), note no significant difference (p=0.5085) in total basal OCR (right) between treatments. B) Real time measurements of extracellular acidification rate (ECAR) from MSC2530818 treated micromasses (left). Note a 37% decrease in total basal glycolysis (inferred from OCR/ECAR measurements) observed compared to DMSO control (right). (all data N=3, error bars=SD, *p=≤0.05).

FIG. 24: Treatment of THP-1 derived macrophages with MSC2530818 (MSC). A) Gene expression of pro-inflammatory cytokines from THP-1 derived macrophages co-treated with 100 nM MSC2530818 (MSC) and LPS for 24 hrs. All pro-inflammatory markers display reduced expression in MSC2530818 (MSC) treated condition compared to DMSO control. Approx. 2-fold reduction in TNFα, approx. 10-fold reduction in IL1β and approx. 10-fold reduction in IL6. B) production of IL1β is reduced by 1.5-fold in MSC2530818 (MSC) treated cells. (all data N=3, error bars=SD, *p=≤0.05, **p=≤0.01. ***p=≤0.001, ****p=≤0.0001)

FIG. 25: Number of hypertrophic cells in MSC2530818 (MSC) treated 5 dpf zebrafish endochondral jaw bone. Images of dissected region of endochondral bone within the zebrafish jaw following treatment with 50 nM or 100 nM MSC2530818 (MSC) or DMSO control (top). Note the dose dependent reduction in the size of the hypertrophic region and corresponding reduction in number of cells (bottom) (all data N=3, error bars=SD. *p=≤0.05. **p=≤0.01)

FIG. 26: Cartilage manifestations and biomarker readout in MSC2530818 (MSC) treated STR/Ort mouse pilot study. Male STR/Ort aged 18 weeks dosed intermittently with MSC2530818 (MSC) 5 mg/kg or DMSO for 2 weeks. A) representative images of articular cartilage post-dissection following DMSO control and MSC2530818 (MSC) treatment. Note the infiltration of blood vessels observed in the DMSO control treated mice which is not present in the MSC2530818 (MSC) treated group. Cartilage in the MSC2530818 (MSC) treated group also appears thicker compared to control. B) Gene expression of cartilage anabolic biomarkers shows increase in expression in MSC treatment group compared to DMSO control. (N=2, data not powered for significance)

FIG. 27: Gait analysis of MSC2530818 (MSC) treated STR/Ort. A) Still captures from video analysis of STR/Ort mouse gait when dosed intermittently with 5 mg/kg MSC2530818 (MSC, top) compared to DMSO control (bottom). B) Number of steps per second from rear left (left), rear right (middle) and left/right ratio (right) with DMSO and MSC2530818 (MSC) treated mice. An increase in steps per limb is observed in MSC2530818 (MSC) treated mice (1.25-fold increase left limb), (1.5-fold increase right limb). MSC2530818 (MSC) treated mice display gait symmetry with step ratio being closer to 1. (error bars=SD, *p=≤0.05, **p=≤0.01, MSC N=6, DMSO N=5).

FIG. 28: MicroCT analysis of subchondral bone of MSC2530818 (MSC) treated STR/Ort. Male STR/Ort aged 22 weeks were dosed intermittently with MSC2530818 (MSC; 5 mg/kg; oral) for 12 weeks. Analysis of manually segmented subchondral bone. Note no difference in bone volume/total volume (left), bone surface density (middle) or bone surface area (right) indicating MSC2530818 (MSC) does not have a bone anabolic or catabolic effect (N=3/group).

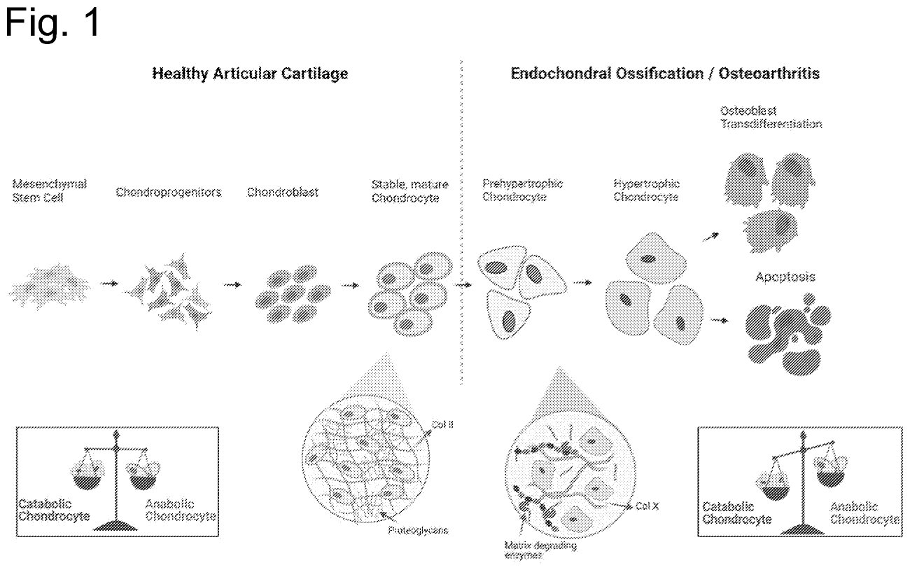

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a Mediator kinase inhibitor” includes “Mediator kinase inhibitors”, reference to “an anabolic mediator” includes two or more such anabolic mediators, and the like.

In general, the term “comprising” is intended to mean including but not limited to. For example, the phrase “a method comprising administering a Mediator kinase inhibitor to the individual” should be interpreted to mean that the method contains a step of administering such an inhibitor, but that the method may contain additional steps such as, for example, administering a further therapeutic agent.

In some aspects of the disclosure, the word “comprising” is replaced with the phrase “consisting of”. The term “consisting of” is intended to be limiting. For example, the phrase “a method consisting of administering a Mediator kinase inhibitor to the individual” should be interpreted to mean that the method contains a step administering such an inhibitor, and no additional steps.

### Treatment of Disorders Characterised by Chondrocyte Hypertrophy

Disclosed herein is a method of treating a disorder characterised by chondrocyte hypertrophy in an individual, comprising administering a Mediator kinase inhibitor to the individual. The disclosure further provides a Mediator kinase inhibitor for use in a method of treating a disorder characterised by chondrocyte hypertrophy in an individual, the method comprising administering the Mediator kinase inhibitor to the individual. The Mediator kinase inhibitor may treat the disorder of chondrocyte hypertrophy by reducing chondrocyte hypertrophy. For instance, the number or proportion of hypertrophic chondrocytes in a given region of cartilage may be reduced.

The utility of Mediator kinase inhibitors in treating disorders characterized by chondrocyte hypertrophy is surprising. Mediator is a conserved multi-subunit protein complex, which is pivotal in many aspects of stimulus-responsive gene expression, including organisation of chromatin structure and regulation of different phases of RNA polymerase II-mediated transcription. Mediator activity is guided by reversible association with a four-subunit kinase module. The kinase module consists of one of the two highly similar cyclin-dependent protein kinase (CDK) paralogues, CDK8 or CDK19. CDK8 or CDK19 is complexed with cyclin C (CCNC), MED12 or 12L, and MED13 or 13L. CDK8 and CDK19 control transcription through phosphorylation of both the C-terminal domain of RNA polymerase II and transcription factors to alter their activity or mark them for degradation.

Accordingly, CDK8 typically exists as part of a multi-protein complex comprising CDK8, CCNC, MED12 and MED13, that associates with the Mediator complex to regulate transcription. Most functions ascribed to the Mediator complex kinase activity are demonstrated to be driven by CDK8.

CDK8 is a known oncogene in certain cancers, such as colorectal cancer. CDK8 inhibitors have thus previously been investigated as a cancer treatment. However, the present inventors have demonstrated for the first time that CDK8 has a role in disorders characterized by chondrocyte hypertrophy, and that Mediator kinase inhibitors (i.e. CDK8 inhibitors) may be used to treat such disorders. Treatment using a CDK8 inhibitor (or CDK8i) is advantageous over existing treatments for disorders characterized by chondrocyte hypertrophy. While existing treatments primarily aim to alleviate symptoms, CDK8 inhibitors instead minimise or stop disease progression. For instance, CDK8 inhibitors may slow the loss of healthy cartilage or the inappropriate formation of bone, reduce pathological bone remodeling in the context of subchondral sclerosis, and/or promote the formation of new healthy cartilage. This could not have been predicted from prior art use of CDK8 inhibitors to treat cancer.

Less is known about CDK19 than CDK8. CDK19 is though known to assemble into a Mediator kinase module analogous to CDK8, and to function in a kinase-independent manner in transcriptional responses to various stimuli. Given certain similarities between CDK8 and CDK19, CDK19 may also have a role in disorders characterized by chondrocyte hypertrophy, and CDK19 inhibitors may be used to treat such disorders. Like treatment using a CDK8 inhibitor, treatment using a CDK19 inhibitor (or CDK19i) may be advantageous over existing treatments for disorders characterized by chondrocyte hypertrophy. While existing treatments primarily aim to alleviate symptoms, CDK19 inhibitors may instead minimise or stop disease progression. For instance, CDK19 inhibitors may slow the loss of healthy cartilage or the inappropriate formation of bone, reduce pathological bone remodeling in the context of subchondral sclerosis, and/or promote the formation of new healthy cartilage.

### Disorder Characterised by Chondrocyte Hypertrophy

The Mediator kinase inhibitor is administered to the individual in order to treat a disorder characterised by chondrocyte hypertrophy. Any disorder characterised by chondrocyte hypertrophy may be treated in this way. Such disorders are well-known in the art.

As explained above, in healthy adult articular cartilage, chondrocytes are maintained in a steady state and do not differentiate into hypertrophic chondrocytes permissive of mineralization and bone formation. In this way, cartilage homeostasis is maintained. This cartilage homeostasis is disrupted in disorders characterised by chondrocyte hypertrophy. In such disorders, chondrocytes differentiate to form hypertrophic chondrocytes, rather than being maintained in a stable, mature form. In essence, the differentiation pathway involved in endochondral ossification is inappropriately followed and leads to the formation of hypertrophic chondrocytes.

Hypertrophic chondrocytes promote the formation of collagen X and a mineralised cartilage matrix, which can be detrimental to the health of the individual. For example, chondrocyte hypertrophy in a joint may lead to the degradation and/or loss of articular cartilage. Chondrocyte hypertrophy in an intervertebral disc may lead to loss of disc flexibility and subsequent narrowing of the gap between adjacent vertebrae. The function of the intervertebral joint may thus be impaired, leading to pain and inflammation. In heterotopic ossification, chondrocyte hypertrophy may lead to the formation of bone tissue outside of the skeleton, for instance in muscle and soft tissues. In this way, chondrocyte hypertrophy may result in the formation of firm, tender swellings which may reduce the range of motion of the joint served by the muscle or soft tissue. In hereditary multiple exostoses, chondrocyte hypertrophy may lead to the development of benign osteocartilaginous masses known as exostoses or osteochondromas. Typically, such masses develop towards the ends of long bones of the limbs or on flat bones, and may disrupt physeal growth, limit the range of motion, and cause joint pain.

Accordingly, the disorder characterised by chondrocyte hypertrophy may be a disorder of chondrogenic differentiation. The disorder characterised by chondrocyte hypertrophy may, for instance, comprise abnormal chondrogenic differentiation. The disorder characterised by chondrocyte hypertrophy may, for instance, comprise abnormal hypertrophic differentiation of chondrocytes. As explained above, chondrogenic differentiation (comprising hypertrophic differentiation of chondrocytes) is an important part of endochondral ossification, the process by which growth cartilage forms bone during skeletal development. Abnormal chondrogenic differentiation may therefore be considered to be chondrogenic differentiation that takes place outside of normal endochondral ossification, and/or outside of skeletal development. Similarly, abnormal hypertrophic differentiation of chondrocytes may be considered to be hypertrophic differentiation of chondrocytes that takes place outside of normal endochondral ossification, and/or outside of skeletal development. The term “normal endochondral ossification” refers to endochondral ossification that occurs during proper development of an individual (for instance, during formation of the skeletal system or growth of long bones), or to maintain the health of an individual (for instance, by healing bone fractures).

The disorder characterised by chondrocyte hypertrophy may, for example, comprise loss of cartilage. The disorder characterised by chondrocyte hypertrophy may, for example, comprise mineralization of cartilage. The disorder characterised by chondrocyte hypertrophy may therefore result in a decrease in the amount (e.g. thickness) of cartilage. The disorder characterised by chondrocyte hypertrophy may result in a decrease in cartilage quality. For instance, cartilage may become more brittle and/or have a reduced shock-absorbing capacity. Accordingly, the disorder characterised by chondrocyte hypertrophy may comprise or consist of cartilage damage. In any case, the cartilage may, for example, be articular cartilage or cartilage comprised in an intervertebral disc. The disorder characterised by chondrocyte hypertrophy may be osteoarthritis or degenerative disc disease, for example.

The disorder characterised by chondrocyte hypertrophy may, for example, comprise formation of bone. The formation of bone may, for instance, be abnormal formation of bone. Abnormal formation of bone may be considered to be the formation of bone outside of normal skeletal development. For instance, abnormal bone formation may result in misshapen bones, for example due to the formation of an exostosis or osteophyte. Abnormal formation of bone may, for instance, result in the production of bone in a non-skeletal tissue, such as a soft tissue. Soft tissues may, for example, include muscle, tendon or ligament. The disorder characterised by chondrocyte hypertrophy may, for example, be heterotopic ossification or hereditary multiple exostoses.

The disorder characterised by chondrocyte hypertrophy may, for example, comprise formation of an osteocartilaginous mass (otherwise known as known as an exostosis or osteochondroma). The disorder characterised by chondrocyte hypertrophy may, for example, be an osteochondroma or hereditary multiple exostoses.

Methods for determining cartilage loss, mineralisation and/or damage are well-known in the art. For example, imaging techniques (such as x-ray, magnetic resonance imaging (MRI) or computed tomography (CT)) may be used to determine joint space narrowing, visualise cartilage, and to determine its amount, shape and degree of mineralisation. Samples may be analysed for the presence of biomarkers indicative of cartilage loss, mineralisation and/or damage. For instance, CTX-II is well-known in the art as a biomarker of cartilage turnover. Elevated levels of CTX-II in a sample obtained from an individual may indicate increased cartilage turnover in the individual, which may be associated with of cartilage loss, mineralisation and/or damage. The sample may, for instance, be a urine sample or a blood sample. Similar techniques may be used to determine bone formation and/or the development of an osteocartilaginous mass.

### Individual

The individual may be any individual that has a disorder characterised by chondrocyte hypertrophy. Such disorders are described in detail above. In one aspect of the disclosure, the individual is an individual in need of cartilage repair and/or inhibition of cartilage degradation.

The individual may, for example, be a mammal. For instance, the individual may be a human. The individual may, for example, be a non-human mammal, such as a dog, cat or horse.

The individual may, for example, be an adult. The individual may, for example, be a juvenile.

### Mediator Kinase Inhibitor

The Mediator kinase inhibitor may be any agent that inhibits a kinase component of the four-subunit kinase module that reversibly associates with Mediator. As set out above, the kinase module consists of:
(i) CDK8 or CDK19;
(ii) cyclin C (CCNC);
(iii) MED12 or 12L; and
(iv) MED13 or 13L.
   Thus, the kinase component may be CDK8 or CDK19.

Accordingly, the Mediator kinase inhibitor may be any agent that inhibits CDK8. The Mediator kinase inhibitor may selectively inhibit CDK8. In the context of the present disclosure, a selective inhibitor of CDK8 is an agent that inhibits CDK8 and has no inhibitory effect (or minimal inhibitory effect) on other CDK enzymes. The Mediator kinase inhibitor may be a CDK8 inhibitor.

The Mediator kinase inhibitor may any agent that inhibits CDK19. The Mediator kinase inhibitor may selectively inhibit CDK19. In the context of the present disclosure, a selective inhibitor of CDK19 is an agent that inhibits CDK19 and has no inhibitory effect (or minimal inhibitory effect) on other CDK enzymes. The Mediator kinase inhibitor may be a CDK19 inhibitor.

The Mediator kinase inhibitor may any agent that inhibits CDK8 and CDK19. The Mediator kinase inhibitor may selectively inhibit CDK8 and CDK19. In the context of the present disclosure, a selective inhibitor of CDK8 and CDK19 is an agent that inhibits CDK8 CDK19, and has no inhibitory effect (or minimal inhibitory effect) on other CDK enzymes. The Mediator kinase inhibitor may be a CDK19 inhibitor.

CDK8 inhibitors and CDK19 inhibitors are described in detail below. The Mediator kinase inhibitor may have any of the properties described below for CDK8 inhibitors or CDK19 inhibitors. For example, the Mediator kinase inhibitor may increase (i.e. promote) expression and/or deposition of one or more cartilage extracellular matrix components. The Mediator kinase inhibitor may increase (i.e. promote) expression of one or more anabolic mediators of cartilage extracellular matrix. The Mediator kinase inhibitor may increase (i.e. promote) expression of one of more transcription factors. The Mediator kinase inhibitor may decrease (i.e. inhibit) expression of one of more transcription factors. The Mediator kinase inhibitor may modulate the activity of one of more transcription factors or signaling proteins by phosphorylation. The Mediator kinase inhibitor may increase (i.e. promote) expression of an inhibitor of a matrix degrading enzyme, and/or (ii) an inhibitor of angiogenesis/hypertrophy. The Mediator kinase inhibitor may increase (i.e. promote) expression of an inhibitor of angiogenesis/hypertrophy. The Mediator kinase inhibitor may reduce mineralisation of cartilage extracellular matrix. That is, the CDK8 inhibitor may reduce the amount of mineral present in the cartilage extracellular matrix. The Mediator kinase inhibitor may decrease expression of one or more promoters of chondrocyte hypertrophy. The Mediator kinase inhibitor may increase expression of one or more inhibitors of chondrocyte hypertrophy. The Mediator kinase inhibitor may decrease expression of one or more promoters of matrix mineralisation. The Mediator kinase inhibitor may increase expression of one or more inhibitors of matrix mineralisation. The Mediator kinase inhibitor may modulate of the expression of protein. The Mediator kinase inhibitor may inhibit osteoblast mineralisation and/or osteoblast differentiation. The Mediator kinase inhibitor may reduce osteoclast resorption. The Mediator kinase inhibitor may have an anti-inflammatory effect. The Mediator kinase inhibitor may exert an anti-inflammatory effect by reducing or eliminating expression of one or more pro-inflammatory mediators, for instance by macrophages such as synovial macrophages. The Mediator kinase inhibitor may exert an anti-inflammatory effect by initiating or increasing expression of one or more anti-inflammatory mediators, for instance by macrophages such as synovial macrophages. The Mediator kinase inhibitor may, for example, have an effect on chondrocyte metabolism. For instance, the Mediator kinase inhibitor may reduce the dependency of chondrocytes on glycolysis for energy production.

The Mediator kinase inhibitor may, for example, have an effect on cartilage, such as articular cartilage, without affecting one or more properties of bone, such as subchondral bone. The effect on cartilage may, for example, be any of the cartilage-related effects described above. The Mediator kinase inhibitor may, for instance have no effect on BV/TV. The Mediator kinase inhibitor may, for instance, have no effect on bone surface density.

The Mediator kinase inhibitor may have a beneficial effect on one or more other properties of bone, such as subchondral bone. For example, as set out above, the Mediator kinase inhibitor may reduce osteoclast resorption. The Mediator kinase inhibitor may therefore have an anti-catabolic effect on bone, such as subchondral bone.

### Cyclin-Dependent Kinase 8 (CDK8) Inhibitor

The CDK8 inhibitor may be any agent that inhibits CDK8. The agent may, for example, selectively inhibit CDK8. In the context of the present disclosure, a selective inhibitor of CDK8 is an agent that inhibits CDK8 and has no inhibitory effect (or minimal inhibitory effect) on other CDK enzymes. Alternatively, the agent may have an inhibitory effect one or more CDK enzymes in addition to CDK8. For instance, the agent may be a CDK8/CDK19 inhibitor.

The CDK8 inhibitor may, for example, impair one or more functions of CDK8. Exemplary CDK8 inhibitors are known in the art, and include BI1347, BRD6989, AS2863619, SEL120-34A, MSC2530818 and CCT251545. The CDK8 inhibitor administered to the individual may therefore comprise or consist of BI1347, BRD6989, AS2863619, SEL120-34A, MSC2530818 or CCT251545, for instance. The CDK8 inhibitor may, for example, comprise or consist of BI1347. The CDK8 inhibitor may, for example, comprise or consist of MSC2530818.

The CDK8 inhibitor may, for example, increase (i.e. promote) expression and/or deposition of one or more cartilage extracellular matrix components. For instance, the CDK8 inhibitor may increase expression and/or deposition of two or more, three or more, four or more or five or more cartilage extracellular matrix components. Components of cartilage extracellular matrix are well-known in the art. The one or more cartilage extracellular matrix components may, for example, comprise collagen. Typically, the collagen is collagen type II (COL2A1). The one or more cartilage extracellular matrix components may, for example, comprise a proteoglycan. The proteoglycan may, for example, comprise one or more glycosaminoglycans, such as hyaluronic acid and/or chondroitin sulfate. The proteoglycan may, for example, comprise aggrecan (ACAN). The one or more cartilage extracellular matrix components may, for example, comprise Martrilin-3 (MATN3). Accordingly, the CDK8 inhibitor may, for example, increase deposition of collagen (such as COLII), a proteoglycan (such as ACAN), MATN3, a glycosaminoglycan, hyaluronic acid and/or chondroitin sulfate, alone or in any combination.

To do so, the CDK8 inhibitor may increase (i.e. promote) expression of one or more anabolic mediators of cartilage extracellular matrix. For instance, the CDK8 inhibitor may increase expression of two or more, three or more, four or more or five or more anabolic mediators of cartilage extracellular matrix. An anabolic mediator of cartilage extracellular matrix may be considered to be an agent that promotes the deposition of one or more cartilage extracellular matrix components. For example, an anabolic mediator of cartilage extracellular matrix may promote the deposition of collagen (such as COLII), a proteoglycan, a glycosaminoglycan, hyaluronic acid and/or chondroitin sulfate, alone or in any combination. Exemplary anabolic mediators of cartilage extracellular matrix are known in the art, and include fibroblast growth factor 18 (FGF-18), insulin like growth factor (IGF-1) and growth differentiation factor 5 (GDF5), and/or their receptors. Typically, anabolic mediators of cartilage extracellular matrix are proteins. Increased expression of an anabolic mediator of cartilage extracellular matrix may, for example, refer to increasing the amount of a nucleic acid (e.g. mRNA) encoding the mediator, and/or increasing the amount of the mediator protein.

The CDK8 inhibitor may, for example, reduce expression of an enzyme that breaks down one or more cartilage extracellular matrix components. For instance, the CDK8 inhibitor may reduce expression of ADAMTS5. ADAMTS5 functions as an aggrecanase, to cleave aggrecan.

The CDK8 inhibitor may, for example, increase (i.e. promote) expression of one of more transcription factors. The transcription factor may be a transcription factor that promotes expression of one or more anabolic mediators of cartilage extracellular matrix. The transcription factor may be a transcription factor that promotes expression and/or deposition of one or more cartilage extracellular matrix components. Anabolic mediators and cartilage extracellular matrix components are described above. The one or more transcription factors may, for example, comprise SOX9, SOX8, SOX6, SOX5. These transcription factors are required for healthy chondrogenesis.

The CDK8 inhibitor may, for example, increase (i.e. promote) expression of an inhibitor of a matrix degrading enzyme, and/or (ii) an inhibitor of angiogenesis/hypertrophy. For instance, the CDK8 inhibitor may increase expression of two or more, three or more, four or more or five or more inhibitors of a matrix degrading enzyme. Inhibitors of matrix degrading enzymes are well known in the art and include, for example, tissue inhibitors of metalloproteinases (TIMPs). TIMPs are a family of four protease inhibitors, namely TIMP1, TIMP2, TIMP3 and TIMP4. The CDK8 inhibitor may increase expression of any one or more of TIMP1, TIMP2, TIMP3 and TIMP4.

The CDK8 inhibitor may, for example, increase (i.e. promote) expression of an inhibitor of angiogenesis/hypertrophy. For instance, the CDK8 inhibitor may increase expression of two or more, three or more, four or more or five or more inhibitors of angiogenesis/hypertrophy. Inhibitors of angiogenesis/hypertrophy are well known in the art and include, for example, chondromodulin-1 (CNMD).

The CDK8 inhibitor may, for example, reduce mineralisation of cartilage extracellular matrix. That is, the CDK8 inhibitor may reduce the amount of mineral present in the cartilage extracellular matrix. The mineral may, for example, comprise calcium. The mineral may, for example, comprise hydroxyapatite. As set out above, mineralization may be determined using imaging methods such as x-ray, MRI or CT. To reduce mineralisation, the CDK8 inhibitor may decrease expression of one or more promoters of chondrocyte hypertrophy. For instance, the CDK8 inhibitor may decrease expression of two or more, three or more, four or more or five or more promoters of chondrocyte hypertrophy. A promoter of chondrocyte hypertrophy may be considered to be an agent that promotes hypertrophic differentiation of chondrocytes. For example, a promoter of chondrocyte hypertrophy may be an agent that positively regulates endochondral ossification. Exemplary promoters of chondrocyte hypertrophy are known in the art, and include bone morphogenetic protein (BMP), transforming growth factor beta (TGF-β) and Wnt. Typically, promoters of chondrocyte hypertrophy are proteins. Increased expression of a promoter of chondrocyte hypertrophy may, for example, refer to increasing the amount of a nucleic acid (e.g. mRNA) encoding the promoter, and/or increasing the amount of the promoter protein.

To reduce mineralisation of cartilage extracellular matrix, the CDK8 inhibitor may increase expression of one or more inhibitors of chondrocyte hypertrophy. For instance, the CDK8 inhibitor may increase expression of two or more, three or more, four or more or five or more inhibitors of chondrocyte hypertrophy. A inhibitor of chondrocyte hypertrophy may be considered to be an agent that inhibits hypertrophic differentiation of chondrocytes. For example, an inhibitor of chondrocyte hypertrophy may be an agent that negatively regulates endochondral ossification. Exemplary inhibitors of chondrocyte hypertrophy are known in the art, and include FGFR3, GDF5, and Chondromodulin. An inhibitor of chondrocyte hypertrophy may be a BMP inhibitor, such as SMOC2. The CDK8 inhibitor may increase expression of SMOC2. Typically, inhibitors of chondrocyte hypertrophy are proteins. Increased expression of a inhibitor of chondrocyte hypertrophy may, for example, refer to increasing the amount of a nucleic acid (e.g. mRNA) encoding the inhibitor, and/or increasing the amount of the inhibitor protein.

To reduce mineralisation, the CDK8 inhibitor may decrease expression of one or more promoters of matrix mineralisation. For instance, the CDK8 inhibitor may decrease expression of two or more, three or more, four or more or five or more promoters of matrix mineralisation. A promoter of matrix mineralisation may be considered to be an agent that promotes mineralisation of the cartilage extracellular matrix. Mineralisation of the cartilage extracellular matrix can be determined using techniques disclosed herein, such as alizarin red staining. Exemplary promoters of matrix mineralisation are known in the art, and include: IBSP, PHOSPHO1, TNAP, COL10A1 and BGLAP. The CDK8 inhibitor may decrease expression of one or more IBSP, PHOSPHO1, TNAP, COL10A1 and BGLAP, in any combination.

To reduce mineralisation of cartilage extracellular matrix, the CDK8 inhibitor may increase expression of one or more inhibitors of matrix mineralisation. For instance, the CDK8 inhibitor may increase expression of two or more, three or more, four or more or five or more inhibitors of matrix mineralisation. An inhibitor of matrix mineralisation may be considered to be an agent that inhibits mineralisation of the cartilage extracellular matrix. Exemplary inhibitors of matrix mineralisation are known in the art, and include MGP, OPN, Enpp1 and ANK. The CDK8 inhibitor may increase expression of one or more of MGP, OPN, Enpp1 and ANK, in any combination

The CDK8 inhibitor may, for example, modulate of the expression of protein. The CDK8 inhibitor may, for example modulate expression of Collagen Type X (ColX) and/or Integrin Binding Sialoprotein (IBSP). For example, the CDK8 inhibitor may decrease expression of Collagen Type X and/or Integrin Binding Sialoprotein. ColX is a well-established marker for hypertrophic chondrocyte differentiation, and its expression may be measured using methods routine in the art such as RNAseq. IBSP a major structural protein of the bone matrix, and its expression may also be measured using methods routine in the art such as RNAseq. By modulating (e.g. decreasing) expression of ColX and/or IBSP, the CDK8 inhibitor may inhibit deposition of an extracellular matrix susceptible to mineralisation. That is, modulating (e.g. decreasing) expression of ColX and/or IBSP may render the matrix less susceptible to mineralisation. The CDK8 inhibitor may, for example, reduce (i.e. inhibit) expression of RUNX2. RUNX2 is a key transcription factor associated with osteoblast differentiation, and its expression and may also be measured using methods routine in the art.

The CDK8 inhibitor may, for example, inhibit osteoblast mineralisation and/or osteoblast differentiation. Osteoblasts are cells that synthesise bone by secreting matrix proteins and transporting mineral into the matrix. Osteoblasts that have encircled themselves with bone matrix eventually differentiate into osteocytes, which regulate the turnover of bone material. Increased osteoblast-mediated bone formation is observed in the later stages of OA, leading to the formation of osteophytes which is associated with debilitating joint pain and limit joint motion. Inhibition of osteoblast-mediated bone formation may therefore be beneficial in the treatment of the disorder characterised by chondrocyte hypertrophy. The CDK8 inhibitor may inhibit osteoblast-mediated bone formation by inhibiting or ablating osteoblast mineralisation. The CDK8 inhibitor may inhibit osteoblast-mediated bone formation by inhibiting or ablating osteoblast differentiation. The CDK8 inhibitor may inhibit osteoblast-mediated bone formation by inhibiting or ablating osteoblast mineralisation and inhibiting or ablating osteoblast differentiation.

The CDK8 inhibitor may, for example, reduce osteoclast resorption. Osteoclasts are cells that break down bone tissue during maintenance, repair and remodeling of bones. An increase in subchondral bone remodelling/sclerosis is observed in the early development of OA, and supports disease progression, and osteoclasts have been shown to degrade cartilage extracellular matrix whilst osteoblasts secrete matrix, as set out above. Inhibition of osteoclast resorption (i.e. bone resorption by osteoclasts) may therefore be beneficial in the treatment of the disorder characterised by chondrocyte hypertrophy. Thus, the CDK8 inhibitor may reduce or eliminate bone resorption mediated by osteoclasts. In other words, the CDK8 inhibitor may reduce or eliminate osteoclastic activity.

The CDK8 inhibitor may, for example, have an anti-inflammatory effect. The anti-inflammatory effect may be systemic or localised within the individual. The anti-inflammatory effect may, for example, be localized to a tissue or organ affected by the disorder characterised by chondrocyte hypertrophy, such as a joint or intervertebral disc. The CDK8 inhibitor may, for example, exert an anti-inflammatory effect by reducing or eliminating expression of one or more pro-inflammatory mediators, for instance by macrophages such as synovial macrophages. For instance, the CDK8 inhibitor may reduce or inhibit expression of two or more, three or more, four or more, five or more, six or more, seven or more, eight or more, nine or more or ten or more pro-inflammatory mediators. Pro-inflammatory mediators are well-known in the art. Pro-inflammatory mediators expressed by macrophages include TLR4 and NFκB. Other pro-inflammatory mediators include TNF-α, IL-1β and IL-6. The CDK8 inhibitor may, for example, exert an anti-inflammatory effect by initiating or increasing expression of one or more anti-inflammatory mediators, for instance by macrophages such as synovial macrophages. For instance, the CDK8 inhibitor may initiate or increase expression of two or more, three or more, four or more, five or more, six or more, seven or more, eight or more, nine or more or ten or more anti-inflammatory mediators. Anti-inflammatory mediators are well-known in the art. Anti-inflammatory mediators expressed by macrophages include PPAR-γ.

The CDK8 inhibitor may, for example, have an effect on chondrocyte metabolism. For instance, the CDK8 inhibitor may reduce the dependency of chondrocytes on glycolysis for energy production. The CDK8 inhibitor may reduce glycolysis in chondrocytes. Glycolysis may be measured by measuring extracellular acidification rate, for instance in a culture of chondrocytes. For instance, glycolysis can be inferred by extracellular acidification rate when OCR is constant. Thus, the CDK8 inhibitor may reduce extracellular acidification rate, for instance in a culture of chondrocytes.

The CDK8 inhibitor may, for example, (a) increase expression of ACAN, (b) increase expression of COL2A1, (c) increase expression of SOX9, (d) increase expression of TIMP4, (e) increase expression of MATN3, (f) increase expression of SMOC2, (g) increase expression of CNMD, (h) decrease expression of IBSP, and/or (i) decrease expression of ADAMTS5. For example, the CDK8 inhibitor may: (a); (b); (c); (d); (e); (f); (g); (h); (i); (a), (b); (a), (c); (a), (d); (a), (e); (a), (f); (a), (g); (a), (h); (a), (i); (b), (c); (b), (d); (b), (e); (b), (f); (b), (g); (b), (h); (b), (i); (c), (d); (c), (e); (c), (f); (c), (g); (c), (h); (c), (i); (d), (e); (d), (f); (d), (g); (d), (h); (d), (i); (e), (f); (e), (g); (e), (h); (e), (i); (f), (g); (f), (h); (f), (i); (g), (h); (g), (i); (h), (i); (a), (b), (c); (a), (b), (d); (a), (b), (e); (a), (b), (f); (a), (b), (g); (a), (b), (h); (a), (b), (i); (a), (c), (d); (a), (c), (e); (a), (c), (f); (a), (c), (g); (a), (c), (h); (a), (c), (i); (a), (d), (e); (a), (d), (f); (a), (d), (g); (a), (d), (h); (a), (d), (i); (a), (e), (f); (a), (e), (g); (a), (e), (h); (a), (e), (i); (a), (f), (g); (a), (f), (h); (a), (f), (i); (a), (g), (h); (a), (g), (i); (a), (h), (i); (b), (c), (d); (b), (c), (e); (b), (c), (f); (b), (c), (g); (b), (c), (h); (b), (c), (i); (b), (d), (e); (b), (d), (f); (b), (d), (g); (b), (d), (h); (b), (d), (i); (b), (e), (f); (b), (e), (g); (b), (e), (h); (b), (e), (i); (b), (f), (g); (b), (f), (h); (b), (f), (i); (b), (g), (h); (b), (g), (i); (b), (h), (i); (c), (d), (e); (c), (d), (f); (c), (d), (g); (c), (d), (h); (c), (d), (i); (c), (e), (f); (c), (e), (g); (c), (e), (h); (c), (e), (i); (c), (f), (g); (c), (f), (h); (c), (f), (i); (c), (g), (h); (c), (g), (i); (c), (h), (i); (d), (e), (f); (d), (e), (g); (d), (e), (h); (d), (e), (i); (d), (f), (g); (d), (f), (h); (d), (f), (i); (d), (g), (h); (d), (g), (i); (d), (h), (i); (e), (f), (g); (e), (f), (h); (e), (f), (i); (e), (g), (h); (e), (g), (i); (c), (h), (i); (f), (g), (h); (f), (g), (i); (f), (h), (i); (g), (h), (i); (a), (b), (c), (d); (a), (b), (c), (e); (a), (b), (c), (f); (a), (b), (c), (g); (a), (b), (c), (h); (a), (b), (c), (i); (a), (b), (d), (e); (a), (b), (d), (f); (a), (b), (d), (g); (a), (b), (d), (h); (a), (b), (d), (i); (a), (b), (e), (f); (a), (b), (e), (g); (a), (b), (e), (h); (a), (b), (e), (i); (a), (b), (f), (g); (a), (b), (f), (h); (a), (b), (f), (i); (a), (b), (g), (h); (a), (b), (g), (i); (a), (b), (h), (i); (a), (c), (d), (e); (a), (c), (d), (f); (a), (c), (d), (g); (a), (c), (d), (h); (a), (c), (d), (i); (a), (c), (e), (f); (a), (c), (e), (g); (a), (c), (e), (h); (a), (c), (e), (i); (a), (c), (f), (g); (a), (c), (f), (h); (a), (c), (f), (i); (a), (c), (g), (h); (a), (c), (g), (i); (a), (c), (h), (i); (a), (d), (e), (f); (a), (d), (e), (g); (a), (d), (e), (h); (a), (d), (e), (i); (a), (d), (f), (g); (a), (d), (f), (h); (a), (d), (f), (i); (a), (d), (g), (h); (a), (d), (g), (i); (a), (d), (h), (i); (a), (e), (f), (g); (a), (e), (f), (h); (a), (e), (f), (i); (a), (e), (g), (h); (a), (e), (g), (i); (a), (e), (h), (i); (a), (f), (g), (h); (a), (f), (g), (i); (a), (f), (h), (i); (a), (g), (h), (i); (b), (c), (d), (e); (b), (c), (d), (f); (b), (c), (d), (g); (b), (c), (d), (h); (b), (c), (d), (i); (b), (c), (e), (f); (b), (c), (e), (g); (b), (c), (e), (h); (b), (c), (e), (i); (b), (c), (f), (g); (b), (c), (f), (h); (b), (c), (f), (i); (b), (c), (g), (h); (b), (c), (g), (i); (b), (c), (h), (i); (b), (d), (e), (f); (b), (d), (e), (g); (b), (d), (e), (h); (b), (d), (e), (i); (b), (d), (f), (g); (b), (d), (f), (h); (b), (d), (f), (i); (b), (d), (g), (h); (b), (d), (g), (i); (b), (d), (h), (i); (b), (e), (f), (g); (b), (e), (f), (h); (b), (e), (f), (i); (b), (e), (g), (h); (b), (e), (g), (i); (b), (e), (h), (i); (b), (f), (g), (h); (b), (f), (g), (i); (b), (f), (h), (i); (b), (g), (h), (i); (c), (d), (e), (f); (c), (d), (e), (g); (c), (d), (e), (h); (c), (d), (e), (i); (c), (d), (f), (g); (c), (d), (f), (h); (c), (d), (f), (i); (c), (d), (g), (h); (c), (d), (g), (i); (c), (d), (h), (i); (c), (e), (f), (g); (c), (e), (f), (h); (c), (e), (f), (i); (c), (e), (g), (h); (c), (e), (g), (i); (c), (e), (h), (i); (c), (f), (g), (h); (c), (f), (g), (i); (c), (f), (h), (i); (c), (g), (h), (i); (d), (e), (f), (g); (d), (e), (f), (h); (d), (e), (f), (i); (d), (e), (g), (h); (d), (e), (g), (i); (d), (e), (h), (i); (d), (f), (g), (h); (d), (f), (g), (i); (d), (f), (h), (i); (d), (g), (h), (i); (e), (f), (g), (h); (e), (f), (g), (i); (e), (f), (h), (i); (e), (g), (h), (i); (f), (g), (h), (i); (a), (b), (c), (d), (e); (a), (b), (c), (d), (f); (a), (b), (c), (d), (g); (a), (b), (c), (d), (h); (a), (b), (c), (d), (i); (a), (b), (c), (e), (f); (a), (b), (c), (e), (g); (a), (b), (c), (e), (h); (a), (b), (c), (e), (i); (a), (b), (c), (f), (g); (a), (b), (c), (f), (h); (a), (b), (c), (f), (i); (a), (b), (c), (g), (h); (a), (b), (c), (g), (i); (a), (b), (c), (h), (i); (a), (b), (d), (e), (f); (a), (b), (d), (e), (g); (a), (b), (d), (e), (h); (a), (b), (d), (e), (i); (a), (b), (d), (f), (g); (a), (b), (d), (f), (h); (a), (b), (d), (f), (i); (a), (b), (d), (g), (h); (a), (b), (d), (g), (i); (a), (b), (d), (h), (i); (a), (b), (e), (f), (g); (a), (b), (e), (f), (h); (a), (b), (e), (f), (i); (a), (b), (e), (g), (h); (a), (b), (e), (g), (i); (a), (b), (e), (h), (i); (a), (b), (f), (g), (h); (a), (b), (f), (g), (i); (a), (b), (f), (h), (i); (a), (b), (g), (h), (i); (a), (c), (d), (e), (f); (a), (c), (d), (e), (g); (a), (c), (d), (e), (h); (a), (c), (d), (e), (i); (a), (c), (d), (f), (g); (a), (c), (d), (f), (h); (a), (c), (d), (f), (i); (a), (c), (d), (g), (h); (a), (c), (d), (g), (i); (a), (c), (d), (h), (i); (a), (c), (e), (f), (g); (a), (c), (e), (f), (h); (a), (c), (e), (f), (i); (a), (c), (e), (g), (h); (a), (c), (e), (g), (i); (a), (c), (e), (h), (i); (a), (c), (f), (g), (h); (a), (c), (f), (g), (i); (a), (c), (f), (h), (i); (a), (c), (g), (h), (i); (a), (d), (e), (f), (g); (a), (d), (e), (f), (h); (a), (d), (e), (f), (i); (a), (d), (e), (g), (h); (a), (d), (e), (g), (i); (a), (d), (e), (h), (i); (a), (d), (f), (g), (h); (a), (d), (f), (g), (i); (a), (d), (f), (h), (i); (a), (d), (g), (h), (i); (a), (e), (f), (g), (h); (a), (e), (f), (g), (i); (a), (e), (f), (h), (i); (a), (e), (g), (h), (i); (a), (f), (g), (h), (i); (b), (c), (d), (e), (f); (b), (c), (d), (e), (g); (b), (c), (d), (e), (h); (b), (c), (d), (e), (i); (b), (c), (d), (f), (g); (b), (c), (d), (f), (h); (b), (c), (d), (f), (i); (b), (c), (d), (g), (h); (b), (c), (d), (g), (i); (b), (c), (d), (h), (i); (b), (c), (e), (f), (g); (b), (c), (e), (f), (h); (b), (c), (e), (f), (i); (b), (c), (e), (g), (h); (b), (c), (e), (g), (i); (b), (c), (e), (h), (i); (b), (c), (f), (g), (h); (b), (c), (f), (g), (i); (b), (c), (f), (h), (i); (b), (c), (g), (h), (i); (b), (d), (e), (f), (g); (b), (d), (e), (f), (h); (b), (d), (e), (f), (i); (b), (d), (e), (g), (h); (b), (d), (e), (g), (i); (b), (d), (e), (h), (i); (b), (d), (f), (g), (h); (b), (d), (f), (g), (i); (b), (d), (f), (h), (i); (b), (d), (g), (h), (i); (b), (e), (f), (g), (h); (b), (e), (f), (g), (i); (b), (e), (f), (h), (i); (b), (e), (g), (h), (i); (b), (f), (g), (h), (i); (c), (d), (e), (f), (g); (c), (d), (e), (f), (h); (c), (d), (e), (f), (i); (c), (d), (e), (g), (h); (c), (d), (e), (g), (i); (c), (d), (e), (h), (i); (c), (d), (f), (g), (h); (c), (d), (f), (g), (i); (c), (d), (f), (h), (i); (c), (d), (g), (h), (i); (c), (e), (f), (g), (h); (c), (e), (f), (g), (i); (c), (e), (f), (h), (i); (c), (e), (g), (h), (i); (c), (f), (g), (h), (i); (d), (e), (f), (g), (h); (d), (e), (f), (g), (i); (d), (e), (f), (h), (i); (d), (e), (g), (h), (i); (d), (f), (g), (h), (i); (e), (f), (g), (h), (i); (a), (b), (c), (d), (e), (f); (a), (b), (c), (d), (e), (g); (a), (b), (c), (d), (e), (h); (a), (b), (c), (d), (e), (i); (a), (b), (c), (d), (f), (g); (a), (b), (c), (d), (f), (h); (a), (b), (c), (d), (f), (i); (a), (b), (c), (d), (g), (h); (a), (b), (c), (d), (g), (i); (a), (b), (c), (d), (h), (i); (a), (b), (c), (e), (f), (g); (a), (b), (c), (e), (f), (h); (a), (b), (c), (e), (f), (i); (a), (b), (c), (e), (g), (h); (a), (b), (c), (e), (g), (i); (a), (b), (c), (e), (h), (i); (a), (b), (c), (f), (g), (h); (a), (b), (c), (f), (g), (i); (a), (b), (c), (f), (h), (i); (a), (b), (c), (g), (h), (i); (a), (b), (d), (e), (f), (g); (a), (b), (d), (e), (f), (h); (a), (b), (d), (e), (f), (i); (a), (b), (d), (e), (g), (h); (a), (b), (d), (e), (g), (i); (a), (b), (d), (e), (h), (i); (a), (b), (d), (f), (g), (h); (a), (b), (d), (f), (g), (i); (a), (b), (d), (f), (h), (i); (a), (b), (d), (g), (h), (i); (a), (b), (e), (f), (g), (h); (a), (b), (e), (f), (g), (i); (a), (b), (e), (f), (h), (i); (a), (b), (e), (g), (h), (i); (a), (b), (f), (g), (h), (i); (a), (c), (d), (e), (f), (g); (a), (c), (d), (e), (f), (h); (a), (c), (d), (e), (f), (i); (a), (c), (d), (e), (g), (h); (a), (c), (d), (e), (g), (i); (a), (c), (d), (e), (h), (i); (a), (c), (d), (f), (g), (h); (a), (c), (d), (f), (g), (i); (a), (c), (d), (f), (h), (i); (a), (c), (d), (g), (h), (i); (a), (c), (e), (f), (g), (h); (a), (c), (e), (f), (g), (i); (a), (c), (e), (f), (h), (i); (a), (c), (e), (g), (h), (i); (a), (c), (f), (g), (h), (i); (a), (d), (e), (f), (g), (h); (a), (d), (e), (f), (g), (i); (a), (d), (e), (f), (h), (i); (a), (d), (e), (g), (h), (i); (a), (d), (f), (g), (h), (i); (a), (e), (f), (g), (h), (i); (b), (c), (d), (e), (f), (g); (b), (c), (d), (e), (f), (h); (b), (c), (d), (e), (f), (i); (b), (c), (d), (e), (g), (h); (b), (c), (d), (e), (g), (i); (b), (c), (d), (e), (h), (i); (b), (c), (d), (f), (g), (h); (b), (c), (d), (f), (g), (i); (b), (c), (d), (f), (h), (i); (b), (c), (d), (g), (h), (i); (b), (c), (e), (f), (g), (h); (b), (c), (e), (f), (g), (i); (b), (c), (e), (f), (h), (i); (b), (c), (e), (g), (h), (i); (b), (c), (f), (g), (h), (i); (b), (d), (e), (f), (g), (h); (b), (d), (e), (f), (g), (i); (b), (d), (e), (f), (h), (i); (b), (d), (e), (g), (h), (i); (b), (d), (f), (g), (h), (i); (b), (e), (f), (g), (h), (i); (c), (d), (e), (f), (g), (h); (c), (d), (e), (f), (g), (i); (c), (d), (e), (f), (h), (i); (c), (d), (e), (g), (h), (i); (c), (d), (f), (g), (h), (i); (c), (e), (f), (g), (h), (i); (d), (e), (f), (g), (h), (i); (a), (b), (c), (d), (e), (f), (g); (a), (b), (c), (d), (e), (f), (h); (a), (b), (c), (d), (e), (f), (i); (a), (b), (c), (d), (e), (g), (h); (a), (b), (c), (d), (e), (g), (i); (a), (b), (c), (d), (e), (h), (i); (a), (b), (c), (d), (f), (g), (h); (a), (b), (c), (d), (f), (g), (i); (a), (b), (c), (d), (f), (h), (i); (a), (b), (c), (d), (g), (h), (i); (a), (b), (c), (e), (f), (g), (h); (a), (b), (c), (e), (f), (g), (i); (a), (b), (c), (e), (f), (h), (i); (a), (b), (c), (e), (g), (h), (i); (a), (b), (c), (f), (g), (h), (i); (a), (b), (d), (e), (f), (g), (h); (a), (b), (d), (e), (f), (g), (i); (a), (b), (d), (e), (f), (h), (i); (a), (b), (d), (e), (g), (h), (i); (a), (b), (d), (f), (g), (h), (i); (a), (b), (e), (f), (g), (h), (i); (a), (c), (d), (e), (f), (g), (h); (a), (c), (d), (e), (f), (g), (i); (a), (c), (d), (e), (f), (h), (i); (a), (c), (d), (e), (g), (h), (i); (a), (c), (d), (f), (g), (h), (i); (a), (c), (e), (f), (g), (h), (i); (a), (d), (e), (f), (g), (h), (i); (b), (c), (d), (e), (f), (g), (h); (b), (c), (d), (e), (f), (g), (i); (b), (c), (d), (e), (f), (h), (i); (b), (c), (d), (e), (g), (h), (i); (b), (c), (d), (f), (g), (h), (i); (b), (c), (e), (f), (g), (h), (i); (b), (d), (e), (f), (g), (h), (i); (c), (d), (e), (f), (g), (h), (i); (a), (b), (c), (d), (e), (f), (g), (h); (a), (b), (c), (d), (e), (f), (g), (i); (a), (b), (c), (d), (e), (f), (h), (i); (a), (b), (c), (d), (e), (g), (h), (i); (a), (b), (c), (d), (f), (g), (h), (i); (a), (b), (c), (e), (f), (g), (h), (i); (a), (b), (d), (e), (f), (g), (h), (i); (a), (c), (d), (e), (f), (g), (h), (i); (b), (c), (d), (e), (f), (g), (h), (i); or (a), (b), (c), (d), (e), (f), (g), (h), (i).

The CDK8 inhibitor may, for example, (i) reduce expression of TNF-α; (ii) reduce expression of IL-1B; (iii) reduce expression of IL-6; (iv) reduce expression of TLR4; (v) reduce expression of NF-κB, and/or (vi) increase expression of PPARγ. For instance, the CDK8 inhibitor may: (i); (ii); (iii); (iv); (v); (vi); (i), (ii); (i), (iii); (i), (iv); (i), (v); (i), (vi); (ii), (iii); (ii), (iv); (ii), (v); (ii), (vi); (iii), (iv); (iii), (v); (iii), (vi); (iv), (v); (iv), (vi); (v), (vi); (i), (ii), (iii); (i), (ii), (iv); (i), (ii), (v); (i), (ii), (vi); (i), (iii), (iv); (i), (iii), (v); (i), (iii), (vi); (i), (iv), (v); (i), (iv), (vi); (i), (v), (vi); (ii), (iii), (iv); (ii), (iii), (v); (ii), (iii), (vi); (ii), (iv), (v); (ii), (iv), (vi); (ii), (v), (vi); (iii), (iv), (v); (iii), (iv), (vi); (iii), (v), (vi); (iv), (v), (vi); (i), (ii), (iii), (iv); (i), (ii), (iii), (v); (i), (ii), (iii), (vi); (i), (ii), (iv), (v); (i), (ii), (iv), (vi); (i), (ii), (v), (vi); (i), (iii), (iv), (v); (i), (iii), (iv), (vi); (i), (iii), (v), (vi); (i), (iv), (v), (vi); (ii), (iii), (iv), (v); (ii), (iii), (iv), (vi); (ii), (iii), (v), (vi); (ii), (iv), (v), (vi); (iii), (iv), (v), (vi); (i), (ii), (iii), (iv), (v); (i), (ii), (iii), (iv), (vi); (i), (ii), (iii), (v), (vi); (i), (ii), (iv), (v), (vi); (i), (iii), (iv), (v), (vi); (ii), (iii), (iv), (v), (vi); or (i), (ii), (iii), (iv), (v), (vi).

The CDK8 inhibitor may, for example, have an effect on cartilage, such as articular cartilage, without affecting one or more properties of bone, such as subchondral bone. The effect on cartilage may, for example, be any of the cartilage-related effects described above. The CDK8 inhibitor may, for instance have no effect on BV/TV. The CDK8 inhibitor may, for instance, have no effect on bone surface density.

The CDK8 inhibitor may have a beneficial effect on one or more other properties of bone, such as subchondral bone. For example, as set out above, the CDK8 inhibitor may reduce osteoclast resorption. The CDK8 inhibitor may therefore have an anti-catabolic effect on bone, such as subchondral bone.

### Cyclin-Dependent Kinase 19 (CDK19) Inhibitor

The CDK19 inhibitor may be any agent that inhibits CDK19. The agent may, for example, selectively inhibit CDK19. In the context of the present disclosure, a selective inhibitor of CDK19 is an agent that inhibits CDK19 and has no inhibitory effect (or minimal inhibitory effect) on other CDK enzymes. Alternatively, the agent may have an inhibitory effect one or more CDK enzymes in addition to CDK19. For instance, the agent may be a CDK8/CDK19 inhibitor.

The CDK19 inhibitor may have any of the functions described above for a CDK8 inhibitor. The CDK19 inhibitor may, for example, increase (i.e. promote) expression and/or deposition of one or more cartilage extracellular matrix components. For instance, the CDK19 inhibitor may increase expression and/or deposition of two or more, three or more, four or more or five or more cartilage extracellular matrix components. Components of cartilage extracellular matrix are well-known in the art. The one or more cartilage extracellular matrix components may, for example, comprise collagen. Typically, the collagen is collagen type II (COLII or COL2A1). The one or more cartilage extracellular matrix components may, for example, comprise a proteoglycan. The proteoglycan may, for example, comprise one or more glycosaminoglycans, such as hyaluronic acid and/or chondroitin sulfate. The proteoglycan may, for example, comprise aggrecan (ACAN). The one or more cartilage extracellular matrix components may, for example, comprise Martrilin-3 (MATN3). Accordingly, the CDK19 inhibitor may, for example, increase deposition of collagen (such as COL2A1), a proteoglycan (such as ACAN), MATN3, a glycosaminoglycan, hyaluronic acid and/or chondroitin sulfate, alone or in any combination.

To do so, the CDK19 inhibitor may increase (i.e. promote) expression of one or more anabolic mediators of cartilage extracellular matrix. For instance, the CDK19 inhibitor may increase expression of two or more, three or more, four or more or five or more anabolic mediators of cartilage extracellular matrix. Anabolic mediators of cartilage extracellular matrix are described in detail above. Typically, anabolic mediators of cartilage extracellular matrix are proteins. Increased expression of an anabolic mediator of cartilage extracellular matrix may, for example, refer to increasing the amount of a nucleic acid (e.g. mRNA) encoding the mediator, and/or increasing the amount of the mediator protein.

The CDK19 inhibitor may, for example, reduce expression of an enzyme that breaks down one or more cartilage extracellular matrix components. For instance, the CDK8 inhibitor may reduce expression of ADAMTS5. ADAMTS5 functions as an aggrecanase, to cleave aggrecan.

The CDK19 inhibitor may, for example, have an effect on cartilage, such as articular cartilage, without affecting one or more properties of bone, such as subchondral bone. The effect on cartilage may, for example, be any of the cartilage-related effects described above. The CDK19 inhibitor may, for instance have no effect on BV/TV. The CDK19 inhibitor may, for instance, have no effect on bone surface density.

The CDK19 inhibitor may have a beneficial effect on one or more other properties of bone, such as subchondral bone. For example, as set out above, the CDK19 inhibitor may reduce osteoclast resorption. The CDK19 inhibitor may therefore have an anti-catabolic effect on bone, such as subchondral bone.

### Administration

The Mediator kinase inhibitor may be administered to the individual by any route. Suitable routes include, but are not limited to, the intravenous, oral/buccal, intra-articular, intra-discal, epidural, intramuscular, subcutaneous, intradermal, transdermal, and intraperitoneal routes. The intra-articular route may be especially suitable when the disorder is osteoarthritis. The intra-discal route may be especially suitable when the disease is degenerative disc disease. In these cases, the Mediator kinase inhibitor may be administered directly to the afflicted joint(s) or disc(s) respectively.

The Mediator kinase inhibitor may be formulated using any suitable method. The Mediator kinase inhibitor may, for example, be comprised in a pharmaceutical composition that comprises a pharmaceutically acceptable carrier and/or excipient. The exact nature of a formulation will depend upon several factors including the inhibitor to be administered and the desired route of administration. Suitable types of formulation are fully described in Remington's Pharmaceutical Sciences, 19th Edition, Mack Publishing Company, Eastern Pennsylvania, USA.

The Mediator kinase inhibitor is administered in a manner compatible with the dosage formulation, and in such an amount that will be therapeutically effective. The amount for a given individual may depend on factors such as the disorder to be treated, the severity or stage of the disorder, the age of the patient and so on. The precise amount of Mediator kinase inhibitor to be administered may thus depend on the judgement of the practitioner and may be peculiar to each subject.

### Miniaturised Model

The ATDC5 cell line is derived from a mouse teratocarcinoma. ATDC5 cells undergo chondrogenic differentiation, mimicking endochondral ossification and, therefore, disorders characterised by chondrocyte hypertrophy. High-density cultures of ATDC5 cells (so-called “micromasses”) have previously been used to model endochondral ossification and disorders characterised by chondrocyte hypertrophy. In such models, micromasses of around 10 μl in volume were cultured in wells of a 24-well plate (such wells having a volume of around 2.4 ml). However, the 24-well plate format is not well-suited to high-throughput applications. Existing ATDC5-based models are not, therefore, optimised for use in screening for modulators of endochondral ossification, such as those which may be used to treat disorders characterised by chondrocyte hypertrophy.

The present inventors have addressed this issue by developing a miniaturised model of endochondral ossification. The miniaturised model may be performed in a well of a 96-microwell plate (such wells having a volume of around 400 μl). The miniaturised model is therefore suited to high-throughput applications, such as screening for compositions that may be used to treat (1) disorders characterised by chondrocyte hypertrophy or (2) a fracture.

The disclosure therefore provides a method of producing a miniaturised model of endochondral ossification, comprising: (a) providing a micromass of ATDC5 cells having a volume of about 1 μl to about 7 μl; (b) culturing the micromass in differentiation medium; and (c) culturing the micromass in mineralisation medium for a further 7 days. The disclosure also provides a miniaturised model of endochondral ossification, producible by the method of the disclosure.

### Provision of a Micromass

The method of producing a miniaturised model of endochondral ossification comprises providing a micromass of ATDC5 cells. The term “micromass” refers to a three-dimensional aggregate of cells having a volume in the order of microlitres. Provision of the ATDC5 cells as a micromass is advantageous, as it mimics the structure of naturally-occurring cartilage and permits the types of cellular interactions found therein.

The micromass has a volume of about 1 μl to about 7 μl. This volume is smaller than that of non-miniaturised ATDC5 micromasses described previously, which have a volume of around 10 μl. The present inventors have identified that micromass having a volume of about 1 μl to about 7 μl is suitable for use in a well of a 96-microwell plate. The present inventors have identified that micromass having a volume of about 8 μl or over is not suitable for use in a well of a standard 96-microwell plate. 96-microwell plates are typically used for high-throughput assays, and comprise 96 individual microwells each with a volume of around 400 μl.

The micromass may, for example, have a volume of about 1 μl, about 1.5 μl, about 2 μl, about 2.5 μl, about 3 μl, about 3.5 μl, about 4 μl, about 4.5 μl, about 5 μl, about 5.5 μl, about 6 μl, about 6.5 μl, or about 7 μl. The micromass may, for example, have a volume of about 1 μl to about 5 μl, such as about 1.5 μl to about 3 μl, or about 2 μl. The inventors have identified that a micromass volume of about 2 μl may be optimal for use in a vessel having a volume of around 400 μl. Thus, a micromass volume of about 2 μl may be optimal for use in a well of a standard 96-well plate.

Non-miniaturised ATDC5 micromasses described previously, having a volume of around 10 μl, typically comprise around 2.7×10⁵ per micromass. The micromass of the present disclosure, having a volume of about 1 μl to about 7 μl, may comprise about 5.0×10⁴ to about 6.0×10⁴ ATDC5 cells, for example. For instance, the micromass may comprise about 5.1×10⁴ ATDC5 cells to about 5.9×10⁴ ATDC5 cells, about 5.2×10⁴ ATDC5 cells to about 5.8×10⁴ ATDC5 cells, about 5.3×10⁴ ATDC5 cells to about 5.7×10⁴ ATDC5 cells, or about 5.4×10⁴ ATDC5 cells to about 5.6×10⁴ ATDC5 cells. The micromass may, for example, comprise about 5.1×10⁴ ATDC5 cells, about 5.2×10⁴ about ATDC5 cells, about 5.3×10⁴ ATDC5 cells, about 5.4×10⁴ ATDC5 cells, about 5.5×10⁴ ATDC5 cells, about 5.6×10⁴ ATDC5 cells, about 5.7×10⁴ ATDC5 cells, about 5.8×10⁴ ATDC5 cells, about 5.9×10⁴ ATDC5 cells or about 6.0×10⁴ ATDC5 cells. For a micromass volume of about 2 μl, 5.4×10⁴ ATDC5 cells are typically used.

### Differentiation Step

The method of producing a miniaturised model of endochondral ossification comprises culturing the micromass in differentiation medium. Differentiation medium is a culture medium specifically designed to trigger normal chondrogenic differentiation of the ATDC5 cells. In other words, differentiation medium promotes the production of mature chondrocytes and extracellular matrix components as found in healthy cartilage.

Differentiation medium may, for example, comprise DMEM-F12 as a base medium. DMEM-F12 is a well-known base medium, and its components are standard in the art. The DMEM-F12 may, for example, be supplemented with one or more of (i) foetal bovine serum (FBS), (ii) an antibiotic-antimycotic composition (Ab/Am), (iii) human transferrin, and (iv) an Insulin-Transferrin-Selenium composition (ITS). The DMEM-F12 may be supplemented with (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv). Preferably, the DMEM-F12 is supplemented with (i), (ii), (iii) and (iv).

In any case, component (i) may, for example, be provided at a concentration of 1% to 10%, such as 2% to 9%, 3% to 8%, 4% to 7%, or 5% to 6%. For instance, component (i) may be provided at a concentration of 1%, 2%, 3%, 4%, 5%, 6%, 7%, 8%, 9% or 10%. Preferably, component (i) is provided at a concentration of 5%. Component (ii) may, for example, be provided at a concentration of 0.2% to 2%, such as 0.5% to 1.5%, or 1%. For instance, component (ii) may be provided at a concentration of 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 0.7%, 1.8%, 1.9% or 2.0%. Preferably, component (ii) is provided at a concentration of 1%. Component (iii) may, for example, be provided at a concentration of lug/ml to 10 μg/ml, such as 2 μg/ml to 9 μg/ml, 3 μg/ml to 8 μg/ml, 4 μg/ml to 7 μg/ml, or 5 μg/ml to 6 μg/ml. For instance, component (iii) may be provided at a concentration of lug/ml, 2 μg/ml, 3 μg/ml, 4 μg/ml, 5 μg/ml, 6 μg/ml, 7 μg/ml, 8 μg/ml, 9 μg/ml or 10 μg/ml. Preferably, component (iii) is provided at a concentration of 5 μg/ml. Component (iv) may, for example, be provided at a concentration of 0.5× to 1.5×, such as 1×. Preferably, component (iv) is provided at a concentration of 1×.

In a preferred aspect, the DMEM-F12 is supplemented with 5% FBS, 1% Ab/Am, 5 μg/ml human transferrin and 1×ITS (10 μg/ml insulin; 5.5 μg/ml transferrin and 6.7 ng/ml sodium selenite).

The volume of differentiation medium in which the micromass is cultured is determined by the vessel in which culture takes place. As mentioned above, the culture vessel is typically a well of 96-microwell plate, in which each well has a volume of about 400 μl. Accordingly, the micromass may, for example, be cultured in about 50 μl medium to about 400 μl medium, such as about 100 μl medium to about 350 μl medium, about 150 μl medium to about 300 μl medium, or about 200 μl medium to about 250 μl medium. For instance, the micromass may be cultured in about 50 μl, about 100 μl, about 150 μl, about 200 μl, about 250 μl, about 300 μl, about 350 μl, or about 400 μl medium.

The micromass may be cultured in differentiation medium for any suitable period of time. The micromass may, for example, be cultured in differentiation medium for at least 3 days. That is, the e micromass may be cultured in differentiation medium for 3 days or more. For instance, the micromass may be cultured in differentiation medium for 3 to 14 days, such as 3 days, 4 days, 5 days, 6 days, 7 days, 8 days, 9 days, 10 day, 11 days, 12 days, 13 days or 14 days. The micromass may, for example, be cultured in differentiation medium for at least 7 days. The micromass may, for example, be cultured in differentiation medium for 7 to 14 days, such as 7 days, 8 days, 9 days, 10 days, 11 days, 12 days, 13 days or 14 days. The inventors have found that the exact length of the period in which the micromass is cultured in differentiation medium is unimportant. A culture period of 3 days, 4 days, 5 days, 6 days, 7 days, 8 days, 9 days, 10 days, 11 days, 12 days, 13 days or 14 days is equally suitable for producing a miniaturised model of endochondral ossification. A shorter culture period may though be preferred, as it allows the method to be performed more quickly. Typically, a culture period of 7 days may be used.

### Mineralisation Step

Following culture in differentiation medium as above, the methods of the disclosure comprise culturing the micromass in mineralisation medium. Mineralisation medium is a culture medium specifically designed to promote the differentiation of mature chondrocytes into hypertrophic chondrocytes, as occurs in endochondral ossification and disorders characterised by chondrocyte hypertrophy.

Mineralisation medium may, for example, comprise Alpha-MEM as a base medium. Alpha-MEM is well-known base medium, and its components are standard in the art. The Alpha-MEM may, for example, be supplemented with one or more of (1) foetal bovine serum (FBS), (2) an antibiotic-antimycotic composition (Ab/Am), (3) human transferrin, (4) an Insulin-Transferrin-Selenium composition (ITS), (5) β-glycerophosphate and (6) ascorbic acid. The Alpha-MEM may be supplemented with (1); (2); (3); (4); (5); (6); (1) and (2); (1) and (3); (1) and (4); (1) and (5); (1) and (6); (2) and (3); (2) and (4); (2) and (5); (2) and (6); (3) and (4); (3) and (5); (3) and (6); (4) and (5); (4) and (6); (5) and (6); (1), (2) and (3); (1), (2) and (4); (1), (2) and (5); (1), (2) and (6); (1), (3) and (4); (1), (3) and (5); (1), (3) and (6); (1), (4) and (5); (1), (4) and (6); (1), (5) and (6); (2), (3) and (4); (2), (3) and (5); (2), (3) and (6); (2), (4) and (5); (2), (4) and (6); (2), (5) and (6); (3), (4) and (5); (3), (4) and (6); (3), (5) and (6); (4), (5) and (6); (1), (2), (3) and (4); (1), (2), (3) and (5); (1), (2), (3) and (6); (1), (2), (4) and (5); (1), (2), (4) and (6); (1), (2), (5) and (6); (1), (3), (4) and (5); (1), (3), (4) and (6); (1), (3), (5) and (6); (1), (4), (5) and (6); (2), (3), (4) and (5); (2), (3), (4) and (6); (2), (3), (5) and (6); (2), (4), (5) and (6); (3), (4), (5) and (6); (1), (2), (3), (4) and (5); (1), (2), (3), (4) and (6); (1), (2), (3), (5) and (6); (1), (2), (4), (5) and (6); (1), (3), (4), (5) and (6); (2), (3), (4), (5) and (6); (1), (2), (3), (4), (5) and (6). Preferably, the Alpha-MEM is supplemented with (1), (2), (3), (4), (5) and (6).

In any case, component (1) may, for example, be provided at a concentration of 1% to 10%, such as 2% to 9%, 3% to 8%, 4% to 7%, or 5% to 6%. For instance, component (1) may be provided at a concentration of 1%, 2%, 3%, 4%, 5%, 6%, 7%, 8%, 9% or 10%. Preferably, component (1) is provided at a concentration of 5%. Component (2) may, for example, be provided at a concentration of 0.2% to 2%, such as 0.5% to 1.5%, or 1%. For instance, component (2) may be provided at a concentration of 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 0.7%, 1.8%, 1.9% or 2.0%. Preferably, component (2) is provided at a concentration of 1%. Component (3) may, for example, be provided at a concentration of lug/ml to 10 μg/ml, such as 2 μg/ml to 9 μg/ml, 3 μg/ml to 8 μg/ml, 4 μg/ml to 7 μg/ml, or 5 μg/ml to 6 μg/ml. For instance, component (3) may be provided at a concentration of lug/ml, 2 μg/ml, 3 μg/ml, 4 μg/ml, 5 μg/ml, 6 μg/ml, 7 μg/ml, 8 μg/ml, 9 μg/ml or 10 μg/ml. Preferably, component (3) is provided at a concentration of 5 μg/ml. Component (4) may, for example, be provided at a concentration of 0.5× to 1.5×, such as 1×. Preferably, component (4) is provided at a concentration of 1×. Component (5) may, for example, be provided at a concentration of 1 mM to 10 mM, such as 2 mM to 9 mM, 3 mM to 8 mM, 4 mM to 7 mM, or 5 mM to 6 mM. For instance, component (5) may be provided at a concentration of 1 mM, 2 mM, 3 mM, 4 mM, 5 mM, 6 mM, 7 mM, 8 mM, 9 mM or 10 mM. Preferably, component (5) is provided at a concentration of 7 mM. Component (6) may, for example, be provided at a concentration of 10 μg/ml to 100 μg/ml, such as 20 μg/ml to 90 μg/ml, 30 μg/ml to 80 μg/ml, 40 μg/ml to 70 μg/ml, or 50 μg/ml to 60 μg/ml. For instance, component (6) may be provided at a concentration of 10 μg/ml, 15 μg/ml, 20 μg/ml, 25 μg/ml, 30 μg/ml, 13 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, or 100 μg/ml. Preferably, component (6) is provided at a concentration of 50 μg/ml.

In a preferred aspect, the Alpha-MEM is supplemented with 5% FBS, 1% Ab/Am, 5 μg/ml human transferrin, 1×ITS, 7 mM β-glycerophosphate, and 50 μg/ml Ascorbic acid.

The volume of mineralisation medium in which the micromass is cultured is determined by the vessel in which culture takes place. As mentioned above, the culture vessel is typically a well of 96-microwell plate, in which each well has a volume of about 400 μl. Accordingly, the micromass may, for example, be cultured in about 50 μl medium to about 400 μl medium, such as about 100 μl medium to about 350 μl medium, about 150 μl medium to about 300 μl medium, or about 200 μl medium to about 250 μl medium. For instance, the micromass may be cultured in about 50 μl, about 100 μl, about 150 μl, about 200 μl, about 250 μl, about 300 μl, about 350 μl, or about 400 μl medium.

The micromass may be cultured in mineralisation medium for any suitable period of time. The micromass may, for example, be cultured in mineralisation medium for at least 2 days. That is, the micromass maybe cultured in mineralisation medium for 2 days or more. For instance, the micromass may be cultured in mineralisation medium for 2 to 14 days, such as 2 days, 3 days, 4 days, 5 days, 6 days, 7 days, 8 days, 9 days, 10 days, 11 days, 12 days, 13 days or 14 days. The micromass may, for example, be cultured in mineralisation medium for at least 7 days. The micromass may, for example, be cultured in mineralisation medium for 7 to 14 days, such as 7 days, 8 days, 9 days, 10 days, 11 days, 12 days, 13 days or 14 days. The micromass may, for example, be cultured in mineralisation medium for 2 to 7 days, such as 2 days, 3 days, 4 days, 5 days, 6 days or 7 days. The inventors have found that the exact length of the period in which the micromass is cultured in mineralisation medium is unimportant. A culture period of 2 days, 3 days, 4 days, 5 days, 6 days, 7 days, 8 days, 9 days, 10 day, 11 days, 12 days, 13 days or 14 days is equally suitable for producing a miniaturised model of endochondral ossification. A shorter culture period may though be preferred, as it allows the method to be performed more quickly.

### Screening Method

The miniaturised model of endochondral ossification described above may be used to screen for compositions that may be used to treat disorders characterised by chondrocyte hypertrophy. The miniaturised model of endochondral ossification described above may be used to screen for compositions that may be used to treat a fracture. The miniaturised model of the disclosure is well-suited to use in such screening, because it may be performed in a well of a standard 96-microwell plate and therefore permit high-throughput screening.

Accordingly, the disclosure provides a method of screening for compositions for use in treating (1) a disorder characterised by chondrocyte hypertrophy or (2) a fracture, comprising: (a) providing a micromass of ATDC5 cells having a volume of about 1 μl to about 7 μl; (b) culturing the micromass in differentiation medium for at least 3 days, wherein a test composition is provided to the micromass on day 1; (c) culturing the micromass in mineralisation medium comprising the test composition for a further 2 days or more; and (d) quantifying the amount of glycosaminoglycans and/or the degree of mineralisation in the micromass following the culture of step (c). In essence, the screening method involves producing the miniaturised model of endochondral ossification in the presence of a test composition (steps (a) to (c)), and determining the effect of the test composition (step (d)).

### Provision of a Micromass

Provision of a micromass is described above in connection with the method of producing a miniaturised model of endochondral ossification. Any of the aspects described above in connection with said method may also apply to step (a) of the screening method.

### Differentiation, Mineralization and Provision of Test Composition

In step (b) of the screening method, the micromass is cultured in differentiation medium for at least 3 days. This differentiation step is described above in connection with the method of producing a miniaturised model of endochondral ossification. Any of the aspects described above in connection with said method may also apply to step (b) of the screening method. However, step (b) of the screening method further comprises providing a test composition to the micromass on day 1 or day 2. In other words, a test composition is provided to the micromass at the start of the period in which the micromass is cultured in differentiation medium, or on the second day of the period in which the micromass is cultured in differentiation medium. The test composition may be comprised in the differentiation medium when the medium is supplied to the micromass. Alternatively, the test composition may be added as a separate reagent to the culture comprising the micromass and the differentiation medium. In any case, the test composition is present in the culture for the duration of the period in which the micromass is cultured in differentiation medium.

In step (c) of the screening method, the micromass is cultured in mineralization medium for a further 2 days or more. This mineralisation step is described above in connection with the method of producing a miniaturised model of endochondral ossification. Any of the aspects described above in connection with said method may also apply to step (c) of the screening method. However, in step (c) of the screening method, the mineralization medium comprises a test composition. The test composition may be comprised in the mineralisation medium when the medium is supplied to the micromass. Alternatively, the test composition may be added as a separate reagent to the culture comprising the micromass and the mineralisation medium. In any case, the test composition is present in the culture for the 7-day period in which the micromass is cultured in mineralization medium.

The requirement for the test composition to be present from the start of the differentiation step (step (b)) is surprising. The present inventors expected that provision of test composition during the mineralisation step (step (c)) would be sufficient to reveal any utility of the test composition in treating disorders characterised by chondrocyte hypertrophy. This is rational because, as set out above, it is the mineralisation step that mimics the pathogenesis of disorders characterised by chondrocyte hypertrophy. Provision of a test composition at the start of the disease process (i.e. at the start of step (c)) would be expected to reveal any potential therapeutic effect over the downstream course of disease. However, this was not the case. Provision of test compositions at the start of step (c) did not reveal any compositions having potential therapeutic effect. Rather, the inventors found that presence of the test composition is required for the entire culture period (i.e. during both the differentiation step (step (b)) and the mineralization step (step (c)) in order for effects to be seen.

The test composition may be any composition which it is desired to screen for utility in treating a disorder characterised by chondrocyte hypertrophy. The test composition may, for example, comprise a small molecule. A small molecule may be defined as a low molecular weight (≤1000 daltons) organic compound that may regulate a biological process. The test composition may, for example, comprise a peptide or protein. The protein, may, for instance, comprise a binding molecule such as an antibody or an antigen-binding fragment thereof. The test composition may, for instance, comprise a nucleic acid. The nucleic acid may, for example, comprise a nucleic acid silencing molecule, such as an antisense oligonucleotide, a siRNA, a shRNA, a miRNA, a mRNA or a CRIPSR guide sequence.

The test composition may be provided in any amount considered to have potential utility in treating a disorder characterised by chondrocyte hypertrophy. Such amounts may be determined by the skilled person using common general knowledge of the type of molecule, its biological activities and so on. A titration of the test composition may be performed to determine the amount needed to have utility in treating a disorder characterised by chondrocyte hypertrophy.

### Determining the Effect of the Test Composition

Step (d) of the screening method relates to determining the effect of the test composition.

When screening for compositions that may be used to treat a disorder characterised by chondrocyte hypertrophy, the micromass is essentially examined to determine whether or not the test compositions has counteracted the effect of the mineralisation medium. A test composition that reduces or prevents the effect of the mineralisation medium may have utility in treating a disorder characterised by chondrocyte hypertrophy. Such disorders are described in detail above.

When screening for compositions that may be used to treat a fracture, the micromass is essentially examined to determine whether or not the test compositions has promoted endochondral ossification. A test composition that promotes endochondral ossification m may have utility in treating a fracture. As explained above, endochondral ossification is the process by which bones repair following fracture. An agent that promotes endochondral ossification may, therefore, promote bone repair and thus healing of a fracture. The fracture may be any kind of fracture, of any bone. The fracture may, for example, be a non-union fracture. In the context of the present disclosure, the term “non-union” may refer to a fracture that fails to heal, or fails to heal in the usual length of time (i.e. has delayed healing).

To determine the effect of the test composition, step (d) may comprise quantifying the amount of proteoglycans and/or glycosaminoglycans in the micromass following the culture of step (c). To determine the effect of the test composition, step (d) may comprise quantifying the degree of mineralisation in the micromass following the culture of step (c). Step (d) may comprise quantifying both the amount of proteoglycans/glycosaminoglycans in the micromass and the degree of mineralisation in the micromass following the culture of step (c).

The amount of proteoglycans and/or glycosaminoglycans in the micromass following the culture of step (c) may indicate the type or composition of the extracellular matrix. In this case, higher amounts of proteoglycans and/or glycosaminoglycans may indicate an extracellular matrix of the type found in healthy cartilage, whereas lower amounts of proteoglycans and/or glycosaminoglycans may indicate an extracellular matrix of the type found in diseased cartilage or during endochondral ossification leading to bone repair. The amount of mineralisation in the micromass following the culture of step (c) may indicate the extent to which the endochondral ossification/osteoarthritis differentiation pathway has progressed. In this case, higher amounts of mineralisation may indicate a greater degree of progression, relating to enhanced endochondral ossification or more advanced disease (e.g. osteoarthritis). Lower amounts of mineralisation may indicate a lesser degree of progression, relating to reduced endochondral ossification or less advanced disease (e.g. osteoarthritis).

Methods for quantifying the amount of proteoglycans/glycosaminoglycans are known in the art. The amount of proteoglycans may, for example, be quantified by gene expression analysis. The amount of proteoglycans/glycosaminoglycans may, for example, be quantified by alcian blue staining as demonstrated in the Examples. Alcian blue is a polyvalent basic dye that is used to stain for acidic polysaccharides. The staining of micromasses with alcian blue is routine in the art and may easily be practiced by the skilled person. To quantify the degree of alcian blue staining (and, therefore, the amount of proteoglycans/glycosaminoglycans in the micromass), absorbance may be measured at 650 nm. Prior to the measurement of absorption, excess alcian blue may be leached from the micromass by contacting the micromass with guanidine hydrochloride (GuHCl). The micromass may, for example, be contacted with 8M GuHCl.

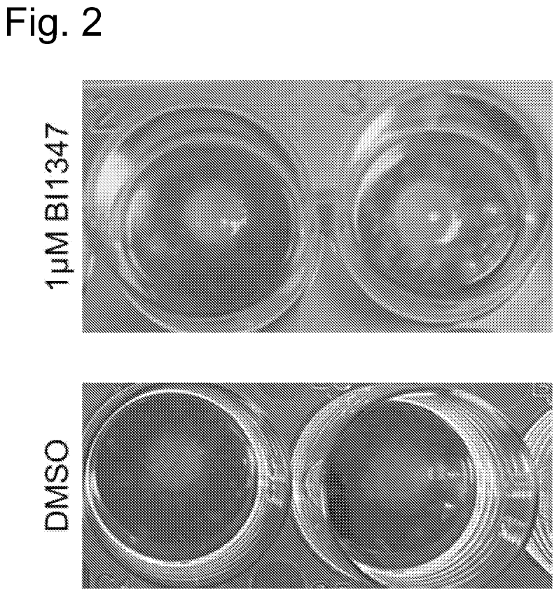

The amount of proteoglycans/glycosaminoglycans may, for example, be quantified by visual inspection of the micromass. That is, the visible phenotype of the micromass may be indicative of the amount of proteoglycans/glycosaminoglycans. The inventors have found that micromasses containing higher amounts of proteoglycans/glycosaminoglycans increase in size (relative to their starting size) from around day 7 of culture. The inventors have also found that micromasses having a higher ratio of proteoglycans/glycosaminoglycans to mineralisation tend to look swollen, rounded and/or shiny. In essence, the appearance of the micromasses containing higher amounts of proteoglycans/glycosaminoglycans to mineralisation closely resembles that of cartilage, as shown in FIG. 2. These visible physical attributes of micromasses having a higher ratio of glycosaminoglycans are thought to be due to increased amounts of proteoglycans/glycosaminoglycans and/or COLII and/or other matrix proteins in the micromass.

Methods for quantifying the degree of mineralization are also known in the art. The degree of mineralization may, for example, be quantified by gene expression analysis. The degree of mineralization may, for example, be quantified by alizarin red staining as demonstrated in the Examples. Alizarin red is a water-soluble sodium salt of Alizarin sulfonic acid that is used to stain for calcium deposits in tissues. The staining of micromasses with alizarin red is routine in the art and may easily be practiced by the skilled person. To quantify the degree of alizarin red staining (and, therefore, the degree of mineralisation in the micromass), absorbance may be measured at 570 nm. Prior to the measurement of absorption, excess alizarin red may be leached from the micromass by contacting the micromass with cetylpyridinium chloride. The micromass may, for example, be contacted with 10% cetylpyridinium chloride.

The degree of mineralisation may, for example, be quantified by visual inspection of the micromass. That is, the visible phenotype of the micromass may be indicative of the degree of mineralisation. The inventors have found that micromasses having a lower degree of mineralisation appear less white and/or less chalky that control masses, once mineralisation has been induced in the culture. These visible physical attributes of micromasses having a lower degree of mineralisation are thought to be due to a reduced amount of ColX and/or other proteins capable of mediating matrix mineralisation (such as PHOSPHO1, BGLAP, IBSP), and/or a reduced amount of mineral deposition, in the micromass.

If it is desired to quantify both the amount of proteoglycans/glycosaminoglycans using alcian blue and the degree of mineralisation using alizarin red, it is typically necessary to perform each stain on a different micromass. To allow for this, steps (a) to (c) of the screening method may be performed in duplicate. The same test composition is used in each of the replicates, at the same concentration. Then, the amount of proteoglycans/glycosaminoglycans may be quantified in one replicate (for instance using alcian blue) and the degree of mineralization may be quantified in a different replicate (for instance using alizarin red). Replicates are performed in separate culture vessels, such as different single microwells. The different single microwells may each be comprised in a different microwell plate, such that one microwell plate is subjected to alcian blue staining and the other alizarin red staining.

A test composition having utility in treating a disorder characterised by chondrocyte hypertrophy may promote an extracellular matrix of the type found in healthy cartilage. This type of extracellular matrix typically contains higher amounts of proteoglycans/glycosaminoglycans. Accordingly, an increase or stabilization in the quantified amount of proteoglycans/glycosaminoglycans may indicate that the test composition has utility in treating the disorder. A test composition having utility in treating a disorder characterised by chondrocyte hypertrophy may reduce or stop progression of the endochondral ossification/osteoarthritis differentiation pathway. Therefore, a decrease in the quantified degree of mineralisation may indicate that the test composition has utility in treating the disorder. Ideally, a test composition having utility in treating a disorder characterised by chondrocyte hypertrophy both promotes an extracellular matrix of the type found in healthy cartilage, and reduces or stop progression of the endochondral ossification/osteoarthritis differentiation pathway. Accordingly an increase or stabilization in the quantified amount of proteoglycans/glycosaminoglycans and a decrease in the quantified degree of mineralisation may indicate that the test composition has utility in treating the disorder.

An increase in the quantified amount of proteoglycans and/or glycosaminoglycans may be an increase relative to the quantified amount of proteoglycans and/or glycosaminoglycans respectively for a negative control composition. A decrease in the quantified degree of mineralisation may be a decrease relative to the quantified degree of mineralization for a negative control composition. The negative control composition may, for example, be DMSO.

A composition that both increases the amount of proteoglycans/glycosaminoglycans and decreases the degree of mineralisation may be particularly beneficial in the treatment of a disorder characterised by chondrocyte hypertrophy. Such a composition may function to both promote the formation or maintenance of healthy cartilage, and reduce or stop disease progression. To assist in identifying such compositions, step (d) may comprise quantifying the amount of proteoglycans/glycosaminoglycans and the degree of mineralisation in the micromass on following the culture of step (c), and calculating the ratio of proteoglycans/glycosaminoglycan production to mineralisation. Such ratio may be calculated by dividing the amount of proteoglycans/glycosaminoglycans by the degree of mineralisation. For instance, if the amount of proteoglycans and the degree of mineralisation is quantified by gene expression analysis, the ratio may be calculated by dividing the measured expression of one or more genes associated with proteoglycan production by the measured expression of one or more genes associated with mineralisation. If the amount of proteoglycans/glycosaminoglycans is quantified by measuring the absorbance of alcian blue staining at 650 nm, and the degree of mineralisation is quantified by measuring the absorbance of alizarin staining at 570 nm, the ratio may be calculated by dividing the measured absorbance at 650 nm by the measured absorbance at 570 nm. The greater the ratio, the more effective the composition in treating a disorder characterised by chondrocyte hypertrophy.

Compositions that markedly reduce the amount of proteoglycans/glycosaminoglycans may be undesirable in the treatment of a disorder characterised by chondrocyte hypertrophy, even if the composition also reduces the degree of mineralisation. The reason for this is that it may be important that a treatment maintains a healthy cartilage extracellular matrix, in order to minimise cartilage loss. This is especially the case in, for example, osteoarthritis and degenerative disc diseases. Accordingly, a reduction in the quantified amount of proteoglycans and/or glycosaminoglycans by over 40% (such as over 50%, over 60%, over 70%, over 80%, over 90%, or over 95%) may indicate that the test composition does not have utility in treating a disorder characterised by chondrocyte hypertrophy, irrespective of the effect of the composition on the degree of mineralisation. The reduction in the quantified amount of proteoglycans and/or glycosaminoglycans may be relative to the quantified amount of proteoglycans and/or glycosaminoglycans respectively for a negative control (such as DMSO).

A test composition having utility in treating a fracture, such as a non-union fracture, may promote an extracellular matrix of the type found during endochondral ossification/bone repair. This type of extracellular matrix typically contains lower amounts of proteoglycans and/or glycosaminoglycans (for instance, relative to mineralization), especially at later stages of fracture repair. Accordingly, a decrease or stablization in the quantified amount of proteoglycans and/or glycosaminoglycans (for instance, relative to mineralization) may indicate that the test composition has utility in treating the fracture. A test composition having utility in treating a fracture may increase or promote progression of the endochondral ossification/osteoarthritis differentiation pathway. Therefore, an increase in the quantified degree of mineralisation may indicate that the test composition has utility in treating a fracture. Ideally, a test composition having utility in treating a fracture both promotes an extracellular matrix of the type found during endochondral ossification/bone repair, and increases or promotes progression of the endochondral ossification/osteoarthritis differentiation pathway. Accordingly a decrease in the quantified amount of proteoglycans and/or glycosaminoglycans and an increase in the quantified degree of mineralisation may indicate that the test composition has utility in treating the fracture.

A decrease in the quantified amount of glycosaminoglycans may be a decrease relative to the quantified amount of glycosaminoglycans for a negative control composition. A decrease in the quantified amount of proteoglycans may be a decrease relative to the quantified amount of proteoglycans for a negative control composition. An increase in the quantified degree of mineralisation may be an increase relative to the quantified degree of mineralization for a negative control composition. The negative control composition may, for example, be DMSO.

A composition that both decreases the amount of proteoglycans/glycosaminoglycans and increases the degree of mineralisation may be particularly beneficial in the treatment of fracture, such as a non-union fracture. Such a composition may function to both promote an extracellular matrix of the type found during endochondral ossification/bone repair, and promote progression of the endochondral ossification/osteoarthritis differentiation pathway. To assist in identifying such compositions, step (d) may comprise quantifying the amount of proteoglycans and/or glycosaminoglycans, and the degree of mineralisation in the micromass, following the culture of step (c), and calculating the ratio of proteoglycan/glycosaminoglycan production to mineralisation. Such ratio may be calculated by dividing the amount of proteoglycans/glycosaminoglycans by the degree of mineralisation. For instance, if the amount of proteoglycans/glycosaminoglycans and the degree of mineralisation is quantified by gene expression analysis, the ratio may be calculated by dividing the measured expression of one or more genes associated with proteoglycan production by the measured expression of one or more genes associated with mineralisation. If the amount of proteoglycans/glycosaminoglycans is quantified by measuring the absorbance of alcian blue staining at 650 nm, and the degree of mineralisation is quantified by measuring the absorbance of alizarin staining at 570 nm, the ratio may be calculated by dividing the measured absorbance at 650 nm by the measured absorbance at 570 nm. The greater the ratio, the more effective the composition in treating a disorder characterised by chondrocyte hypertrophy.

### Assay Design

As mentioned above, the screening method is typically conducted in a well of a microwell plate. That is, the culture vessel may be a well of a microwell plate. The microwell plate may, for example be a 96-microwell plate and/or a microwell plate in which each well has a volume of around 400 μl. The benefit of using such microwell plate is that the screening method can be conducted as a high-throughput assay.

In the screening method, each of steps (a) to (d) may be conducted in a single culture vessel. Thus, each of steps (a) to (d) may be conducted in a single well of a microwell plate. The microwell plate may, for example, be a microwell plate comprising 96 microwells. The microwell plate may, for example, be a microwell plate in which each well has a volume of around 400 μl.

Steps (a) to (d) of the screening method may be repeated in parallel, in order to screen two or more test compositions at the same time. In other words, steps (a) to (d) may be conducted in multiple different culture vessels at the same time, with a different test composition used in one or more of the different culture vessels. The different culture vessels may be different single microwells comprised in the same microwell plate. Accordingly, steps (a) to (d) may be repeated in parallel, wherein (i) each repeat is conducted in a different single well of the same microwell plate and (ii) a different test composition is provided to the micromass in each repeat. The microwell plate may, for example, be a microwell plate comprising 96 microwells. The microwell plate may, for example, be a microwell plate in which each well has a volume of around 400 μl.

### Composition Identified by the Method

The disclosure further provides a composition for use in treating a disorder characterised by chondrocyte hypertrophy, identified by the screening method of the disclosure.

The composition may be any composition for which it is desired to screen for utility in treating a disorder characterised by chondrocyte hypertrophy. The composition may, for example, comprise a small molecule. The composition may, for example, comprise a peptide or protein. The protein, may, for instance, comprise a binding molecule such as an antibody or an antigen-binding fragment thereof. The composition may, for instance, comprise a nucleic acid. The nucleic acid may, for example, comprise a nucleic acid silencing molecule, such as an antisense oligonucleotide, a siRNA, a shRNA, a miRNA, mRNA or a CRIPSR guide sequence.

The following Examples illustrate the invention.

### Example

### Methods

### Miniaturised, High-Throughput Screening Platform for Chondrogenic Cells Aiding Therapeutic Discovery

An epigenetic library consisting of 226 known epigenetic modulator compounds was used to screen against ATDC5 high density micromass cultures to identify any compounds that have an inhibitory effect on matrix mineralisation (catabolic chondrocyte) whilst maintaining and/or promoting healthy extracellular matrix deposition (anabolic chondrocyte). The opposite may be used for identifying molecules that accelerate endochondral ossification for fracture repair i.e. promotes matrix mineralisation.

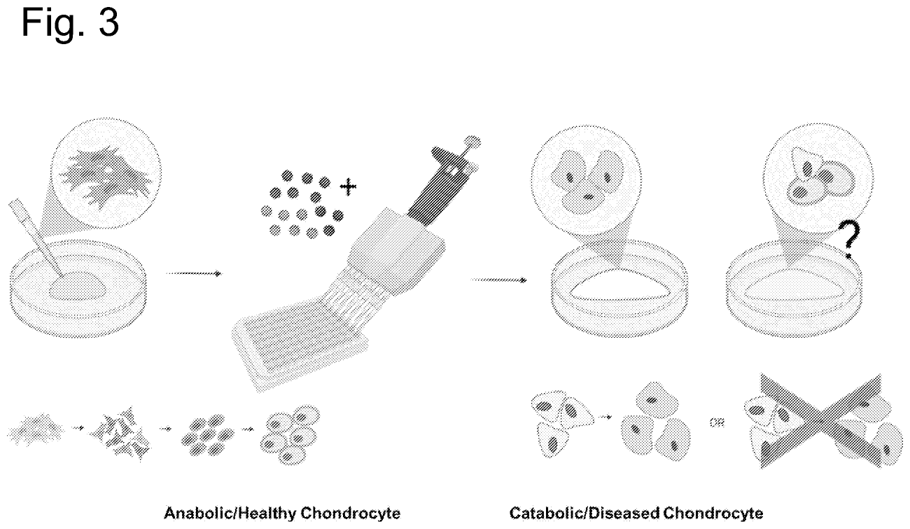

The developed screening method miniaturises the culture whilst shortening the culture period offering a high throughput method for compound screening on chondrogenic cultures. The methodology is shown as a schematic in FIG. 3. Standard micromass cultures consist of a 10 μl droplet of 2.7×10⁵ cells in a 24-well plate, with cells differentiated for 14 days in differentiation media (DMEM-F12, 5% Foetal Bovine Serum (FBS), 1% Antibiotic-Antimycotic (Ab/Am) (Gibco), 5 μg/ml human transferrin, 1× Insulin-Transferrin-Selenium premix (ITS)) and a further 7 days in mineralisation media (Alpha-MEM, 5% FBS, 1% Ab/Am, 7 mM β-glycerophosphate, 5 μg/ml human transferrin, 1×ITS) to induce chondrocyte hypertrophy (Castaño Betancourt, Cailotto et al. 2012)

In the developed screening method, 5.4×10⁴ cells in a total volume of 2 μl standard growth media (DMEM-F12, 5% FBS, 1% Ab/Am 10 μg/ml human transferrin, 3×10⁻⁸ M sodium selenite) were seeded onto single wells of a 96-well plate and incubated at 37° C., 5% CO₂ in a humidified incubator for 2 hours to allow for cell adherence. 100 μl of differentiation media DMEM-F12, 5% FBS, 1% Ab/Am, 5 μg/ml human transferrin, 1×ITS) containing a single compound was added to each well.

Plates were seeded in duplicate to allow for observation each compound has on glycosaminoglycan and mineral deposition at the end of the culture. This enabled for one 96-well plate to screen up to 96 compounds individually. Multiple plates were set up at the same time allowing the entire library to be screened in parallel. Cultures were then differentiated in differentiation media in the presence of the library for 7 days with media changes containing library every 2-3 days. On day 7, media was switched to a mineralisation media (Alpha-MEM, 5% FBS, 1% Ab/Am, 7 mM-GP, 50 μg/ml ascorbic acid, 5 μg/ml human transferrin, 1× Insulin-Transferrin-Selenium premix) and again supplemented with the library for 7 days with media changes every 2-3 days.

On day 14, culture plates were fixed with 95% ice-cold methanol for 25 minutes and each compound treated well stained with either 1% alcian blue pH 2.5, a stain for glycosaminoglycans indicative of healthy ECM or 1% alizarin red pH 4.2, a stain for mineralisation (calcium) indicative of chondrocyte hypertrophy observed in cartilage disease. Alcian blue stained wells were leached with 8M guanidine hydrochloride and alizarin red leached with 10% cetylpyridinium chloride to quantify staining by absorbance 650 nm and 570 nm, respectively. Leached stain absorbances were plotted on GraphPad Prism for each compound for both alizarin red and alcian blue. To identify hits, the alcian blue:alizarin red ratio was plotted and readings higher than the control group ratios were classified as hits. To prevent data skewing, alcian blue absorbances below 60% of the control mean were set to 0 in order to identify compounds that maintain/promote healthy extracellular matrix production and reduced mineralisation simultaneously. Hits were cross referenced with images of stained micromasses to confirm reduction in alizarin red and maintenance/enhancement of alcian blue. This lead to the identification of BI1347, a cyclin-dependent kinase 8 (CDK8) inhibitor (CDK8i), as a strong candidate to take forward for further characterisation.

Preliminary screening efforts were carried out whereby micromasses were seeded as above and differentiated in differentiation media for 14 days. On day 14 the compound library was added to cells and the media was switched to mineralisation media for 7 days to induce hypertrophic differentiation. This initial screening method revealed no compound in the library to have an effect on hypertrophic differentiation, indicating that an earlier time point for compound addition was required in order to identify novel modulators via this screening technique.

### Isolation and Culture of Primary Chondrocytes: Equine, Canine and Human

Articular cartilage was isolated, placed into a petri dish containing phosphate buffer saline (PBS) supplemented with 2% Ab/Am (PBS-Ab/Am) and transferred to a laminar flow hood for processing. Cartilage shavings were minced using a sterile scalpel to approximately 1 mm³ and washed in fresh PBS-Ab/Am by centrifugation at 500×g for 10 minutes. Supernatant was removed and the cartilage pellet resuspended in 5 ml TrypLE™ (Gibco) before incubation at 37° C. for 10 minutes with gentle agitation. Dissociated cartilage fragments were pelleted and washed to remove excess TrypLE and resuspended in DMEM-complete media (DMEM/F12 (Gibco), 1% Ab/Am, 10% FBS) supplemented with 2 mg/ml Collagenase Type II and incubated overnight at 37° C. with gentle agitation. The cartilage digest was pelleted, washed and subsequently resuspended in DMEM-complete. Released cells were counted using a haematocytometer and seeded at 10,000 cells per cm². The cultures were incubated at 37° C., 5% CO₂ in a humidified incubator for 2 days before refreshing DMEM-complete every 2 days until confluency. For differentiation experiments, primary chondrocytes were differentiated in DMEM/F12 containing 2% FBS, 1% Ab/Am, 1× Insulin-Transferrin-Selenium premix, 10 ng/ml TGFβ3 (primary chondrocyte differentiation media).

### Dose Response of BI1347 and Additional CDK8 Inhibitors

To observe whether the effects observed on chondrogenesis was specific to BI1347 or a class effect of CDK8 inhibition, several CDK8 inhibitors were tested in a dose response assay. In brief, 2 μl micromasses were seeded in a 96-well plate and cultured as previous in the presence of BI1347 and additional CDK8i compounds namely BRD6989, AS2863619, SEL120-34A, MSC2530818 and CCT251545. Compound concentrations were titrated 10-fold from 10 μM down to 0.1 nM. On day 14 micromasses were fixed, stained and subsequently leached to allow for dose response analysis to be conducted. This assay allowed for identification of the MSC2530818 as an additional compound take forward.

### Micromass Cultures of CDK8i Treated Chondrogenic Cells for Gene Expression Analysis

ATDC5 and primary equine chondrocytes were cultured in micromasses and treated with a CDK8i to determine effects on gene expression during chondrogenic differentiation.

In more detail, for ATDC5 cells, 2.7×10⁵ cells were seeded in a 10 μl droplet onto a 24-well plate and cultured in differentiation media supplemented with CDK8i for 7 days before switching to mineralisation media supplemented with CDK8i for a further 7 days to induce hypertrophic differentiation as previous. The CDK8i was either 1 μM BI1347 or 100 nM MSC2530818.

On days 7 and 14 DMSO control and BI1347/MSC2530818 treated ATDC5 micromasses were lysed in triplicate and RNA extracted using Reliaprep RNA miniprep system (Promega) before cDNA synthesis using High-capacity reverse transcriptase kit (Applied BioScience). Quantitative PCR (qPCR) analysis was carried out using 10 ng cDNA in iTaq mastermix (BioRad) according to manufacturer's instructions. Primer sequences for qPCR reactions are shown in Table 1. ΔCt values of genes expressed were normalised to reference housekeeper genes and subsequently used to generate relative expression. Significance was observed by conducting unpaired T-test with Welch correction.

For primary equine chondrocytes, chondrocytes were seeded in 10 μl micromasses as above and differentiated for 14 days in presence of primary chondrocyte differentiation media supplemented with 1 μM BI1347 before processing for gene expression analysis as described above.

TABLE 1
qPCR Primer Sequences

| Target Gene | Forward Primer | Reverse Primer |
|---|---|---|
| Equine | GGCAAAACAATGCAAACCTT | CAAGGGCATATCCTACGACAA |
| HPRTI | | |
| Equine ACAN | GAGGAGATGGAGGGTGAGGT | GATGGTGATGTCCTCCTCGC |
| Equine SOX9 | CTGGAGACTGCTGAACGAGA | GAGATGTGTGTCTGCTCCGT |
| Equine | TCCTGGTGTCAAAGGTCACA | TCCCTTAGCACCATCCAGAC |
| COL2A1 | | |
| Equine | ATAGGCTCAGTCGGGGCTC | CCGGATTGCAAGCGCC |
| TIMP4 | | |
| Equine | GGGGAGCGACAATCACATTT | TCTCTCCTCCAGCAAAACGG |
| CNMD | | |
| Equine | TTGGTACACACGGCTGTCAA | CGCACCTGTTACGAACTGAA |
| MATN3 | | |
| Equine | GCACCTACAGTCAGGTCCAG | AGGCATCATCTGTTTTTCCTGT |
| SMOC2 | | |
| Equine | TGGCTCACGAAATTGGACATC | AGGTCTAGCAAACAGTTACCATGACC |
| ADAMTS5 | | |
| Equine | CAGACCAGCAGCACTCCATA | GCAGCATTCTGGAAGGAGAC |
| RUNX2 | | |
| Mouse | CTGGTGAAAAGGACCTCTCGAA | CTGAAGTACTCATTATAGTCAAGGGCAT |
| HPRTI | | |
| Mouse TBP | GAAGAACAATCCAGACTAGCAGCA | CCTTATAGGGAACTTCACATCACAG |
| Mouse PPIA | CGCGTCTCCTTCGAGCTGTTTG | TGTAAAGTCACCACCCTGGCACAT |
| Mouse ACAN | GCTGCAGTGATCTCAGAAGAAG | GATGGTGAGGGAAGACCCTA |
| Mouse SOX9 | GCTGGAAGTCGGAGAGCCGAGA | AGAGAACGAAACCGGGGCCAC |
| Mouse | CTGCCAGTGGAAAATTAGGG | TTCTCCCTTGTCACCACGAT |
| COL2A1 | | |
| Mouse | CCTGGTTCATGGGATGTTTT | CAGGAATGCCTTGTTCTCCT |
| COL10A1 | | |
| Mouse | TCTCCCGCATCATCGACACT | GTCGGAATAGGTGTTGAGCTG |
| MATN3 | | |
| Mouse | CCTGAGGACGTTGAGTTTTGC | CAGCTCCTACCTTGAGCAGC |
| CNMD | | |
| Mouse | CACTCGGCTCTAGTGATACGG | CTTGGCCTTCTCGAACCCTTT |
| TIMP4 | | |
| Mouse | AAGATATTGCCTCACGCTACCC | TCCTCAAGAGCTGACTGATGC |
| SMOC2 | | |
| Mouse | GGAGCGAGGCCATTTACAAC | CGTAGACAAGGTAGCCCACTTT |
| ADAMTS5 | | |
| Mouse IBSP | ATGGAGACGGCGATAGTTCC | CTAGCTGTTACACCCGAGAGT |
| Mouse CDK8 | CGGGTCGAGGACCTGTTTG | TGCCGACATAGAAATTCCAGTTC |
| Mouse CCNC | GTGAATGATACCTACAGGACGGA | GCAAACCACTGTCTAGCATCT |
| Mouse | TTGAGCCAACAGCCTTTCTT | TCATGCATTAGCTGCTTTGG |
| MED12 | | |
| Mouse | ATTACAAGGGTGTTCGAGA | TATAACCTCTCTTGCTGCCAGA |
| MED23 | | |
| Mouse | CTTTGGGGGAGACTATGGTGG | TCGAGCCAGGTGACAAACTC |
| MED25 | | |
| Mouse | TCCGGATTCTCACCAATCGAA | CGATTTCCAGCATCATCTGTCTC |
| MED31 | | |

### IL-1β Induced Extracellular Matrix Degeneration Model

IL-1β is a potent inducer of cartilage degradation and its expression is shown to be significantly upregulated in the synovial fluid of patients with OA and degenerative disc disease (Gorth, Shapiro et al. 2019, Vincent 2019).

To mimic this route of cartilage degradation in vitro, 10 μl ATDC5 micromasses were seeded and differentiated in the presence of 1 μM BI1347 or DMSO for 11 days to allow for sufficient ECM deposition. Cells were then serum starved for 24 hours in differentiation media containing 1% FBS before treatment with 1 μM BI1347 or DMSO for 1 hour and subsequent induction of ECM degradation by co-treatment with 10 ng/ml IL-1β (PeproTech) for 48 hours. Compound only conditions were used as a negative control and additional micromasses were differentiated in the presence of 1 μM BI1347 for the entire culture period to observe whether pre-treatment of ATDC5 with BI1347 alters cellular responses to IL-1B. On day 14 micromasses were stained with alcian blue and leached as previous and RNA extracted for gene expression analysis as above.

Additional experiments were carried out on primary chondrocytes (equine, canine and human) and ihMSC (TERT immortalised human mesenchymal stromal cell line) to investigate whether CDK8i protects from IL-1β induced degradation when added throughout the entirety of differentiation/IL-1β treatment. Briefly, ihMSCs were seeded at a density of 5×10⁴ cells per 10 μl micromass and differentiated in ihMSC chondrogenic media (1% FBS, 1% Ab/Am, 1× Insulin-Transferrin-Selenium premix, 10 μM Y-27632, 100 nM dexamethasone, 50 μg/ml ascorbic acid, 20 ng/ml TGF-β3 (Peprotech)). Primary chondrocytes were seeded at 2.7×10⁵ cells per 10 μl micromass and differentiated in primary chondrocyte differentiation media. All micromasses were differentiated for 12 days in the presence of 100 nM MSC2350818 or DMSO. Some micromasses were processed for gene expression analysis/alcian blue staining on day 12, whilst others were treated with IL-β media (1% FBS, 1% Ab/Am, 10 ng/ml IL-1B) and CDK8i for 48-hours before processing for alcian blue staining/gene expression analysis on day 14 as previously described.

Primer sequences for qPCR reactions are shown in Table 2.

TABLE 2
qPCR Primer Sequences

| Target Gene | Forward Primer | Reverse Primer |
|---|---|---|
| Equine | GGCAAAACAATGCAAACCTT | CAAGGGCATATCCTACGACAA |
| HPRTI | | |
| Equine ACAN | GAGGAGATGGAGGGTGAGGT | GATGGTGATGTCCTCCTCGC |
| Equine SOX9 | CTGGAGACTGCTGAACGAGA | GAGATGTGTGTCTGCTCCGT |
| Equine | TCCTGGTGTCAAAGGTCACA | TCCCTTAGCACCATCCAGAC |
| COL2A1 | | |
| Equine | ATAGGCTCAGTCGGGGCTC | CCGGATTGCAAGCGCC |
| TIMP4 | | |
| Equine | GGGGAGCGACAATCACATTT | TCTCTCCTCCAGCAAAACGG |
| CNMD | | |
| Equine | TTGGTACACACGGCTGTCAA | CGCACCTGTTACGAACTGAA |
| MATN3 | | |
| Equine | GCACCTACAGTCAGGTCCAG | AGGCATCATCTGTTTTTCCTGT |
| SMOC2 | | |
| Equine | TGGCTCACGAAATTGGACATC | AGGTCTAGCAAACAGTTACCATGACC |
| ADAMTS5 | | |
| Equine | CAGACCAGCAGCACTCCATA | GCAGCATTCTGGAAGGAGAC |
| RUNX2 | | |
| Mouse | CTGGTGAAAAGGACCTCTCGAA | CTGAAGTACTCATTATAGTCAAGG |
| HPRTI | | GCAT |
| Mouse | TCCGGATTCTCACCAATCGAA | CGATTTCCAGCATCATCTGTCTC |
| MED31 | | |
| Canine | AGCTTGCTGGTGAAAAGGAC | TTATAGTCAAGGGCATATCC |
| HPRTI | | |
| Canine | TCCACAGCCTATCCAGAACA | CTGCTGCTGTTGTCTCTGCT |
| TBP | | |
| Canine | GCAGCAAGAGCAAGGAC | TTCTGAGAGCCCTCGGT |
| COL2A1 | | |
| Canine | GGACACTCCTTGCAATTTGAG | GTCATTCCACTCTCCCTTCTC |
| ACAN | | |
| Canine | GATGGGTGAGGCGTGCAG | GGATCTGGACTGGCAGGAAC |
| TIMP4 | | |
| Canine | CGGAGGAGCAAAGTGCTACA | GTTGTCCTTCACAGGCTGGT |
| CNMD | | |
| Canine | GGCACAGGAAAAACAGATGATGC | CTTGTTCGGTCCAAAGCGTG |
| SMOC2 | | |
| Canine | ACTGTGAATGCTACGAGGGC | AACAGTCGCAGTGATAGGCG |
| MATN3 | | |
| Canine | TGTCTGACCAAGAAGCTGCC | GCAAACTGTACTCCTCCCCC |
| ADAMTS5 | | |
| Human | TGAGGATTTGGAAAGGGTGT | GAGCACACAGAGGGCTACAA |
| HPRTI | | |
| Human | CCGCCGGCTGTTTAACTTCG | GCGCTGGAACTCGTCTCACT |
| TBP | | |
| Human | ACTGGCGAGCACTGTAACAT | AGCATGTGAAAGAGTCGATGGC |
| ACAN | | |
| Human | TCTCCCGGATAATCGACACTC | CAAGGGTGTGATTCGACCCA |
| MATN3 | | |
| Human | ATGGCAGACGTTGGGACCAT | AATCGTCATGGGAGAGGCCAA |
| ADAMTS5 | | |
| Human | ATGACGACGGCACCTACAG | TCGCGTTGGGGTAACTTTTCA |
| SMOC2 | | |
| Human | AGGCGGTGCTTGTTCCTC | GTTCGAGAAGATGATCTGACTGCC |
| TNF-α | | |
| Human | AGCTACGAATCTCCGACCAC | CGTTATCCCATGTGTCGAAGAA |
| IL-1β | | |
| Human | ACTCACCTCTTCAGAACGAATTG | CCATCTTTGGAAGGTTCAGGTTG |
| IL-6 | | |
| Human | ATAGCGAGCCACGCATTCACA | TGCCAGCTCTTCTGTGTGGTTT |
| TLR4 | | |
| Human | AAGAGGAGGTTTCGCCACCG | AAGGGCAGGGGAAGCTGTCA |
| NF-κβ | | |
| Human | AGCCTGCATTTCTGCATTCTG | TGGCATCTCTGTGTCAACCA |
| PPARγ | | |

### Dose Response of BI1347 and Additional CDK8 Inhibitors

To observe whether the effects observed on chondrogenesis was specific to BI1347 or a class effect of CDK8 inhibition, several CDK8 inhibitors were tested in a dose response assay. In brief, 2 μl micromasses were seeded on a 96-well plate and cultured as previous in the presence of BI1347 and additional Mediator kinase inhibitor compounds namely BRD6989, AS2863619, SEL120-34A, MSC2530818 and CCT251545. Compound concentrations were titrated 10-fold from 10 μM down to 0.1 nM. On day 14 micromasses were fixed, stained and subsequently leached to allow for dose response analysis to be conducted.

### Osteoclast Activity

Osteoclasts degrade bone through resorptive activity and subsequently initiate bone remodelling. An increase in subchondral bone remodelling is observed in the early development of OA supporting disease progression. Additionally, osteoclasts have been shown to degrade cartilage ECM in vitro (Löfvall, Newbould et al. 2018). Inhibition of osteoclast activity via bisphosphonate treatment has been shown to reduce remodelling and slow disease progression (Fernández-Martin, López-Peña et al. 2021). We therefore wanted to observe the effect of CDK8 inhibition via BI1347 treatment on osteoclast function.

Osteoclasts were isolated from the bone marrow of the long bones of 8-week old C57/BL-6 mice and cultured for 24 hours in osteoclast basal media (MEM supplemented with 10% FBS, 2 mM L-glutamine, 1% Ab/Am). After 24 hours, 1×10⁶ osteoclasts were seeded onto dentin discs in osteoclast differentiation media (basal media supplemented with 100 nM PGE2, 200 ng/ml MCSF, 3 ng/ml RANKL pH 7.3) for 24 hours before addition of 1 μM BI1347 or DMSO for 3 days (N=8). On day 5 of culture, osteoclast resorption was initiated by addition of 10 mM hydrochloric acid to acidify differentiation media to pH 7.0 for 2 days. On day 7, discs were fixed in 2.5% glutaraldehyde, tartrate resistant acid phosphatase (TRAP) stained and imaged using Axiovert Microscope at ×10 magnification to observe osteoclast resorption.

Pre-osteoclast and osteoclast numbers were counted using the automated counting method developed by Davies et al. 2021 (currently under review) using Ilastik software in Image J (Fiji, US). Resorption area on each disc was manually counted and analysed using Image J software.

### Osteoblast Activity and Viability

Osteoblasts synthesise bone matrix, which is subsequently mineralised during the bone remodelling process. Increased osteoblast mediated bone formation is observed in the later stages of OA leading to the formation of osteophytes which is associated with debilitating joint pain and limit joint motion (Maruotti, Corrado et al. 2017). It was therefore essential to ensure that CDK8 inhibition via BI1347 did not promote osteoblast mineralisation.

2×10⁴ MC3T3 (subclone 14) osteoblast-like cells were seeded on a 24-well plate and cultured for 2 days in osteoblast growth media (Alpha-MEM supplemented with 10% FBS, 1% Ab/Am) before induction of differentiation with osteoblast differentiation media (Alpha-MEM, 10% FBS, 1% Ab/Am, 50 μg/ml ascorbic acid, 5 mM β-glycerophosphate) in the presence of 1 μM BI1347 or DMSO, with media changes every 2-3 days.

Another MC3T3 differentiation method was also carried out with MSC2530818 treatment. Briefly, MC3T3 cells were seeded as above and differentiated for 7 days in osteogenic induction media (Alpha-MEM, 10% FBS, 1% Ab/Am, 50 μg/ml ascorbic acid) with DMSO or 100 nM MSC2530818, before switching to osteogenic induction media with 2 mM β-glycerophosphate in the presence of 100 nM MSC2530818 or DMSO.

Cells were fixed in 95% ice-cold methanol and stained with 1% Alizarin Red on day 7, 10 and 14 of culture for BI1347 and on day 14 only for MSC2530818 treated cells to observe effects of compounds on mineralization compared to DMSO control. Alizarin red stain was leached and measured as previously described.

To determine potential toxicity of CDK8i on MC3T3 cells, a cell viability assay was carried out on day 7 and 14 of MC3T3 cells treated with 1 μM BI1347. Briefly, culture media was replaced with differentiation media containing 0.2 mM resazurin dye and incubated for 2 hours at 37° C. before measurement at 570 nm normalized to 600 nm.

### Macrophage Inflammatory Response

Macrophages are one of the predominant immune cells present in the synovium and regulate both pro-inflammatory and anti-inflammatory responses within the joint. It is well documented that during OA and IVD, macrophages exacerbate disease progression through upregulation of pro-inflammatory pathways whilst anti-inflammatory mechanisms are downregulated. Additionally, CDK8 inhibition has been shown to successfully dampen pro-inflammatory responses both in vitro and in vivo). We therefore wanted to confirm this is also true with BI1347 and MSC2530818 treatment as this would enhance its therapeutic profile.

THP-1 monocytes were differentiated into macrophages via treatment with 50 ng/ml phorbol 12-myristate 13-acetate (PMA) in THP-1 growth media (RPMI-1640, 100 μg/ml Primocin, 10% FBS, 0.05 mM β-mercaptoethanol) for 48 hours. Macrophages were then washed twice to remove PMA containing media before a 1-hour pretreatment with 1 μM BI1347 or 100 nM MSC2530818. Inflammatory stimulation was then initiated via the addition of lug/ml lipopolysaccharide (LPS) for 24 hours in the presence of 1 μM BI1347 or 100 nM MSC2530818. Gene expression analysis was subsequently carried out as previously outlined. For MSC2530818 treated/LPS stimulated cells, media supernatant was used for IL-1β cytokine analysis quantification via ELISA (PeproTech, ABTS Human IL-1β development ELISA) as per manufacturer instructions.

### RNA Sequencing and Analysis

2.7×10⁵ ATDC5 cells were seeded in a 10 μl micromasses and differentiated in the presence of 1 μM BI1347 or DMSO control for 7 days before RNA extraction as previously described. Two wells were pooled per extraction and treatment conditions were set up in triplicate.

Total RNA quantification and integrity was determined via Bioanalyzer 2100 (Agilent) and RNA samples with an integrity number above 9.0 were processed for RNA sequencing.

The RNA library was formed by polyA capture using poly-T oligo attached magnetic beads and subsequently reverse transcribed for cDNA synthesis and library construction. The library was then sequenced using the Illumina PE150 technology and clean reads with high quality data were taken forward for downstream analysis. Paired-end clean reads were aligned to the Mus Musculus reference genome and reads numbers mapped to each gene. Fragments Per Kilobase of transcript sequence per Millions base pairs sequenced (FPKM) of each gene was calculated based on the length of the gene and reads count mapped to the gene.

Differential expression analysis of BI1347 treatment versus DMSO control was performed. Genes with an adjusted P-value≤0.05 and a fold change≥2 were considered differentially expressed.

Analysis of Gene Ontology for biological processes was carried out using ShinyGO 0.76 with a false discover rate (FDR) cut-off set to 0.05 for both upregulated and downregulated genes individually.

Cluster analysis was carried out on all the differentially expressed genes in each group and biological replicate using hierarchical clustering to cluster the FPKM values of genes.

Genes of interest relating to chondrocyte hypertrophy and cartilage homeostasis were identified.

### Dose Response of CDK8i on Genes of Interest

To confirm that the gene expression profile observed in the day 7 RNAseq dataset was reproducible in hypertrophic cells, we selected genes of interest from the RNAseq dataset and analysed gene expression analysis on hypertrophic ATDC5 cells. 2.7×10⁵ ATDC5 cells were cultured in 10 μl micromasses in the presence of BI1347 and MSC2530818 at concentrations 1 μM, 100 nM, 10 nM and DMSO control for 7 days in differentiation media before switching to mineralisation media for 7 days. On day 14 cells were processed for qPCR analysis as previously described.

### Phased Dosing of B11347 and MSC2350818 on ATDC5 Micromasses

To understand the longevity of the CDK8i effect observed with BI1347 and MSC2530818, ATDC5 micromasses were exposed to a phased dosing regimen. This consisted of treating standard 10 μl ATDC5 micromasses with differentiation media containing 1 μM of BI1347 or MSC2530818 for 2 days before switching to control differentiation media for the remaining culture period ending on day 14 of differentiation. This was repeated for 4, 6, 9, 11 and 14 days of treatment. On day 14, cells were fixed and stained with alizarin red or alcian blue as previously described.

### Metabolic Analysis

Metabolic dysfunction in chondrocytes has been shown to support OA development with increased glycolysis observed within diseased tissue. This higher dependency on glycolysis for energy production is linked to enhanced catabolic processes, therefore reducing glycolysis has been proposed as a therapeutic approach for OA (Tan et al. (2022) “A new strategy for osteoarthritis therapy: Inhibition of glycolysis”. Front Pharmacol. 2022 Nov. 10; 13:1057229. doi: 10.3389/fphar.2022.1057229.). To assess energy metabolism of hypertrophic ATDC5 cells in the presence of CDK8i, 2 μl micromasses were seeded onto a Seahorse XF HS Miniplate (Agilent). Micromasses were differentiated in the presence of MSC2530818 as previously described for 1 week before switching to mineralisation media containing MSC2530818 for 24 hours. The MitoStress Test (Agilent) was carried out on hypertrophic micromasses using 1.5 μM oligomycin, 0.5 μM rotenone A and 0.25 μM FCCP which measured real-time extracellular acidification and oxygen consumption rate. Analysis via the Agilent Seahorse Analytics platform basal oxygen consumption rate and glycolysis measurements.

### In Vivo: Zebrafish Jaw Analysis of Hypertrophic Zones

Several bones during zebrafish (Danio rerio) craniofacial development exist via endochondral ossification, thus the developing zebrafish embryo is a useful in vivo model of chondrocyte hypertrophy (Dietrich et al. (2021) “Skeletal Biology and Disease Modeling in Zebrafish”. J Bone Miner Res, 36:436-458. https://doi.org/10.1002/jbmr.4256). Embryos at 24 hpf were cultured in the E3 water (mM NaCl, 0.17 mM KCl, 0.33 mM CaCl2, 0.33 mM MgSO4, and 0.1% Methylene Blue) in the presence of 50 nM or 100 nM MSC2530818 or DMSO control up until 5 dpf, with E3 water refreshed every 2 days. At 5 dpf embryos were fixed in 4% PFA overnight at 4° C. before washing in 70% EtOH for 10 mins followed by 50% EtOH for a further 10 mins. Specimens were then incubated in alcian blue stain (0.1% Alcian blue, 70% EtOH, 30% Glacial acetic acid) overnight with gentle agitation before 30 min washes in 100, 80, 70% EtOH and 0.1% PBST. Specimens were digested for 1 hour in enzyme solution (10 mg/ml trypsin in 30% saturated sodium tetraborate) followed by a 1-hour incubation in bleaching solution (3% hydrogen peroxide, 1% potassium hydroxide). Specimens were washed in 0.1% PBST and stored in 70% glycerol at 4° C. until imaging. Jaw sections were dissected and the number of enlarged cells within the entopterygoid/ceratohyal region were counted.

### Pilot In Vivo Study for Gene Expression Markers

The STR/Ort mouse is a spontaneous mouse model of osteoarthritis which follows a similar trajectory as primary osteoarthritis disease development as humans and companion animals. Of note, all males develop disease with age and do not require experimental initiation making the STR/Ort mouse a good in vivo osteoarthritis model (Staines et al., 2017). An initial pilot study consisting of 2 male STR/Ort mice aged 18 weeks per group was carried out. Mice were dosed 5 mg/kg MSC2350818 (N=2) or DMSO (N=2) in 60% PEG400 via oral gavage 3 times a week intermittently for 2 weeks. On day 14, mice were sacrificed, and articular cartilage was harvested from the knee joint using a micro rongeur. Tissue was homogenised in Qiazol using 2.4 mm beads in pre-chilled Tissue Lyser II racks (Qiagen) at 30 Hz for 5 minutes in total with intervals for samples to be chilled on ice every 60 seconds to prevent sample heating. Homogenised samples were directly processed for RNA extraction using the Direct-Zol microprep kit (Zymo) and RNA was processed for gene expression analysis via qPCR as previous.

### Str/ORT In Vivo 12 Week Study

Following the pilot study, a larger in vivo study using the STR/Ort OA mouse model was conducted. Str/ORT male mice aged 22 weeks (post OA onset) were dosed with either DMSO (N=5) or MSC2530818 5 mg/kg (N=6) intermittently as above for 12 weeks.

On week 11 mice were placed on the DigiGate™ system (MouseSpecifics), a treadmill based video gait analysis software. For Gait analysis, each mouse was placed on the DigiGate™ and allowed to acclimatise for 5 minutes, the belt speed was then set to 13 cm/s for 10 seconds to capture videos of mice whilst walking. Videos were then cropped to 5 seconds of active walking and number of steps per hind limb was counted for each mouse.

On week 12 mice were sacrificed and hind limbs were fixed in 4% PFA for 48 hours before storing in 70% EtOH. Micro computed tomography (μCT) scans were carried out on hind limbs to analyse MSC2530818 effects on bone. Samples were wrapped in plastic film before scanning to prevent drying and scanned using the Skyscan 1172F (Bruker). Whole limbs were scanned with X-ray settings 50 kV and 200 μA, using an aluminum 0.5 mm filter, 960 ms exposure time and 5 μm pixel size. Scanned images were reconstructed into tomograms using NRecon 1.7.3.1 (Bruker) using a reconstruction threshold with a minimum value of −1000 and maximum value of 9486 in Hounsfield units and repositioned using Dataviewer 1.5.4 (Bruker). Tibial subchondral bone regions were manually segmented and analysed using CTAn 1.18.4 (Bruker).

### Results

### BI1347 Identified as a Modulator of Endochondral Ossification in a Novel Minaturised High-Throughput Screen

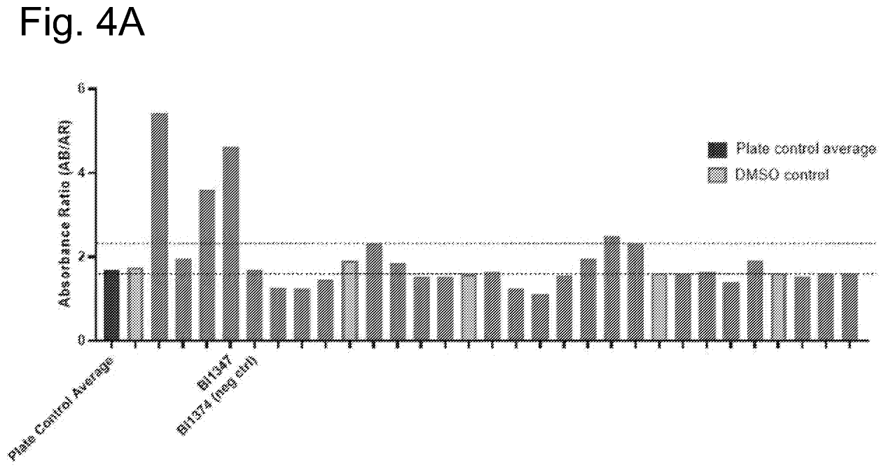

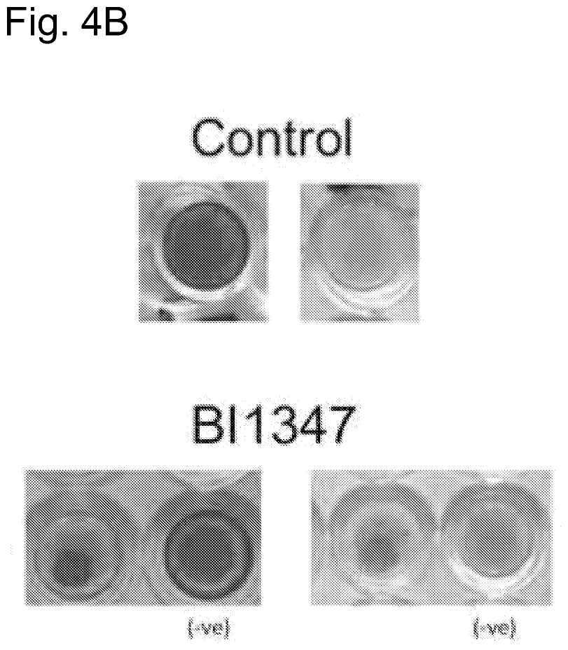

Screening an epigenetic library on the miniaturised endochondral ossification model using ATDC5 micromasses revealed BI1347, a CDK8 inhibitor, as a compound which promotes glycosaminoglycan (GAG) deposition and reduces mineralisation when compared to its negative control BI1374 and DMSO vehicle control (FIG. 4).

Interestingly, changes in GAG deposition were not seen when BI1347 was provided to a monolayer culture of ATDC5 cells. A micromass allows ATDC5 cells to form contacts and relationships that are similar to those found in cartilage tissue. By permitting such contacts and relationships, a micromass provides a more biologically relevant model of cartilage formation/endochondral ossification than does a monolayer. This emphasises the importance of using a micromass for screening. Had only a monolayer system been used, an effect for BI1347 would not have been observed, and CDK8 inhibitors would not have been identified as modulators of endochondral ossification.

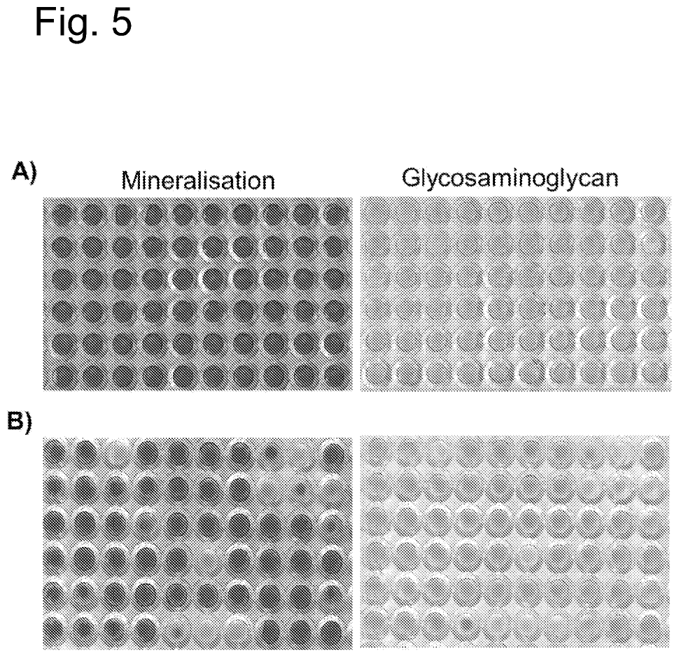

Initial efforts to screen for potential modulators of chondrocyte hypertrophy included differentiating the micromasses for 14 days and adding the compound library for the mineralisation period (days 14-21). This proved to be inadequate as no compound in the library had any phenotypic effect on the micromasses during this window (FIG. 5). When adding the compound library on day 0, at the point of differentiation, notable phenotypic changes were observed across the screening plates indicating that adding the compound at this time point is essential in revealing modulators of endochondral ossification using this system. It is expected that similar results would also be achieved by adding the compound library 1 or 2 days later.

Typically, micromasses were cultured in differentiation medium for 7 days, and then in mineralisation medium for 7 days, as set out in the “Methods” section above. Changes in matrix staining in compound-treated micromasses were though seen after just 3 days in differentiation medium+compound. Therefore, effects (such as anabolic effects) on the micromass may be determined after three or more days, and/or the time in differentiation medium could be reduced to 3 or more days.

Typically, micromasses were cultured in mineralisation medium for 7 days, as set out in the “Methods” section above. Mineralisation were though seen after just 2 days in mineralisation medium. Therefore, mineralisation may be determined after 2 or more days, and/or the time in mineralisation medium could be reduced to 2 or more days.

### CDK8 Inhibition via BI1347 Treatment Promotes Gene Expression of Healthy Cartilage Extracellular Matrix Components and Inhibits COL10A1 Expression In Vitro

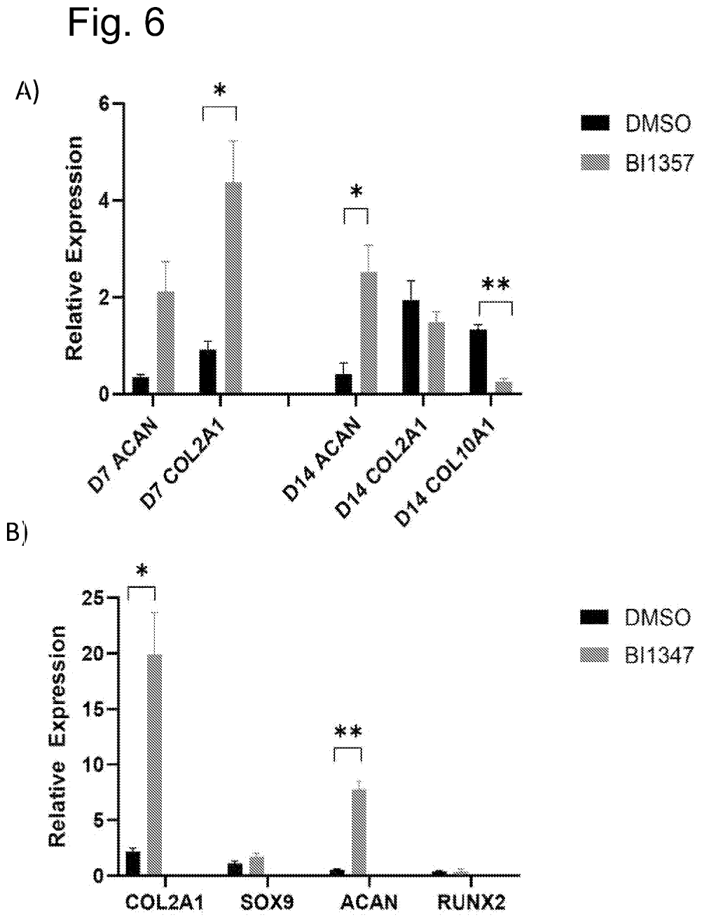

Treatment of ATDC5 micromasses with 1 μM BI1347 for 14 days increased both ACAN (6-fold N.S) and COL2A1 (4.7-fold p≤0.05) expression on day 7 of culture compared to DMSO control (FIG. 6). On day 7, ATDC5 cells are in the peak of anabolic chondrogenesis, thus suggesting that CDK8 inhibition during this stage of differentiation promotes gene expression of essential components of healthy extracellular matrix such as COL2A1 which encodes collagen type 2 and ACAN which encodes the proteoglycan aggrecan. Furthermore, when ATDC5 are pushed into a hypertrophic state via the addition of mineralisation media for a further 7 days, this 6-fold increase in ACAN gene expression is maintained whilst COL2A1 expression resembles that similar to DMSO control on day 14. Most striking is the inhibitory effect CDK8 inhibition via BI1347 has on COL10A1 expression on day 14 with a 5.3-fold (p≤0.01) reduction observed compared to DMSO treated micromasses. It is key to note that throughout the ATDC5 micromass culture, BI1347 treated micromasses were visibly larger, more rounded and resembled the appearance of healthy cartilage. This was not observed in the DMSO control conditions (FIG. 2).

More profound effects on anabolic chondrogenic gene expression were observed when primary equine chondrocytes were treated with 1 μM BI1347. A 9-fold increase in COL2A1 (p≤0.05) and 14-fold increase in ACAN (p≤0.01) is observed at day 14 of culture in the presence of BI1347, indicating CDK8 inhibition dramatically enhances extracellular matrix (ECM) production in primary equine cells in vitro. Furthermore, a 1.5-fold increase (N.S) in SOX9 is observed. SOX9 is required for healthy chondrogenesis and therefore it is essential that its expression is not negatively affected by CDK8 inhibition. Importantly, there is no increase in RUNX2 gene expression, a key transcription factor involved in hypertrophic differentiation.

### CDK8 Inhibition via B11347 or MSC2530818 Treatment Plays a Protective Role in Extracellular Matrix Degradation

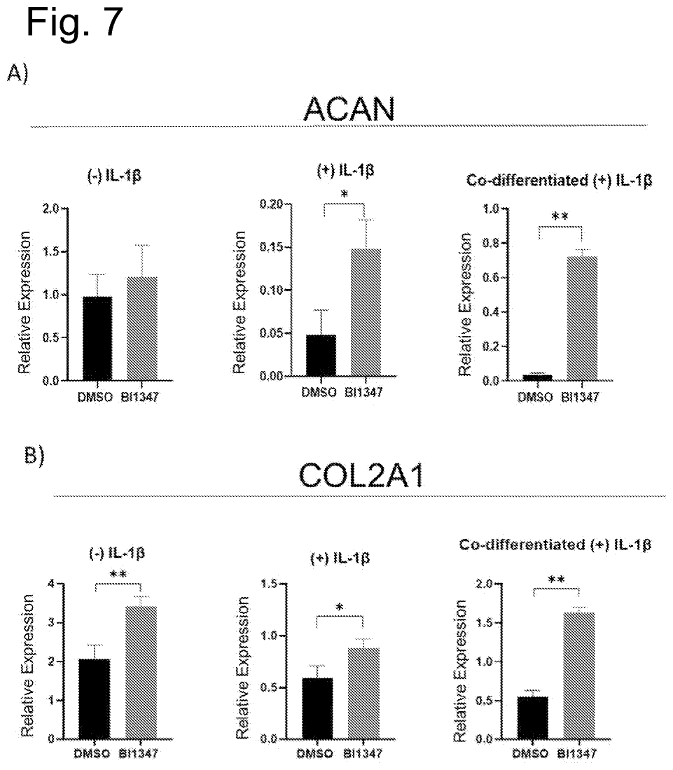

To mimic ECM degradation in vitro, ATDC5 micromasses were differentiated for 11 days to allow for sufficient ECM production before treatment with IL-1β (after serum starvation) for 48 hrs to induce degradation. When 1 μM BI1347 is co-treated with IL-1β on days 12-14, a 3-fold increase in ACAN (p≤0.05) and 1.5-fold increase in COL2A1 (p≤0.05) is observed compared to DMSO control, suggesting CDK8 inhibition via BI1347 may limit ECM degeneration by IL-β induced degradation (FIG. 7). BI1347 treatment alone on day 12-14, for a total of 48 hours, promotes COL2A1 gene expression 1.7-fold (p≤0.05) and ACAN 1.2-fold (N.S) suggesting that BI1347 treatment after a short time frame in pre-differentiated cells is still effective at promoting anabolic chondrogenesis. Most notably, treatment of ATDC5 micromasses with 1 μM BI1347 throughout differentiation and during IL-1ß induced degradation protects ATDC5 cells from cellular responses to IL-1β as both ACAN and COL2A1 expression are maintained. A 3-fold decrease in COL2A1 (p≤0.01) and a 20-fold decrease in ACAN (p≤0.01) is observed in DMSO control compared to micromasses co-differentiated with 1 μM BI1347 in this IL-1β treated condition, indicating that IL-1β alone significantly reduces anabolic chondrogenesis and CDK8 inhibition prevents this.

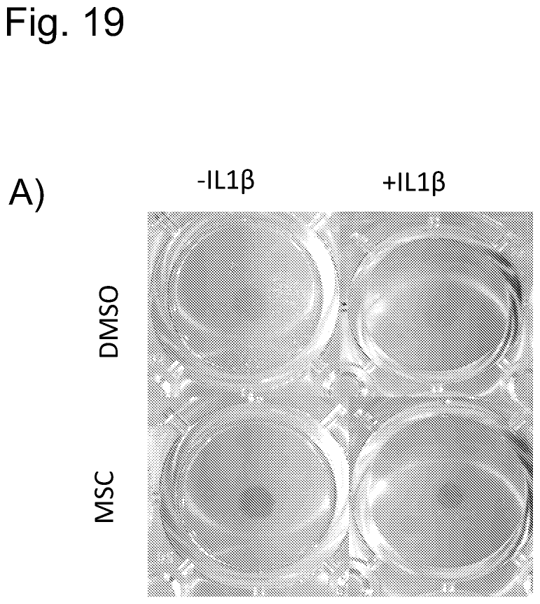

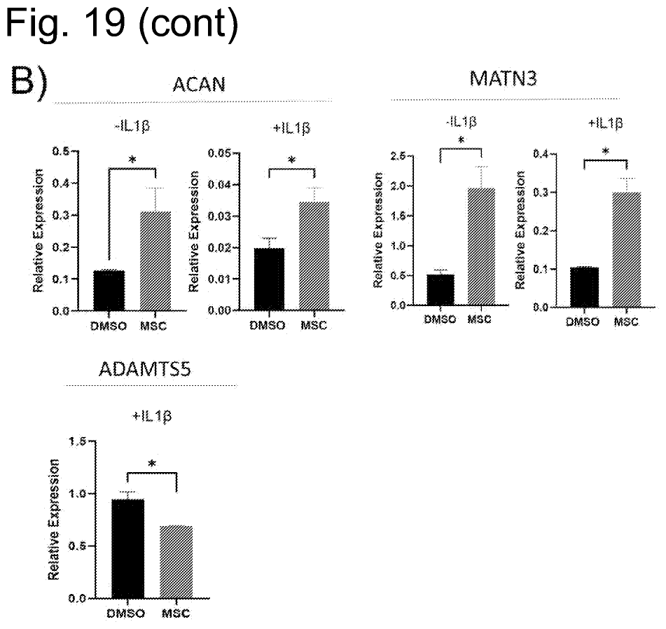

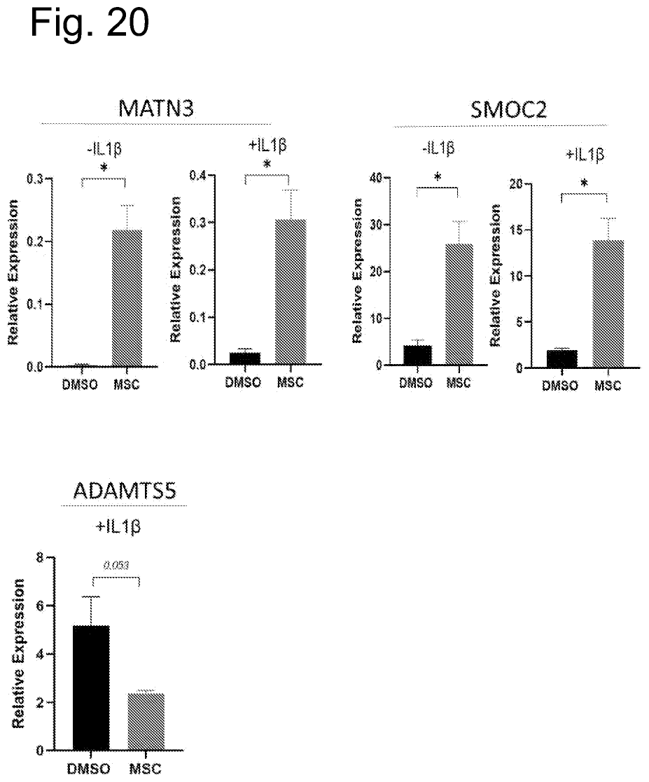

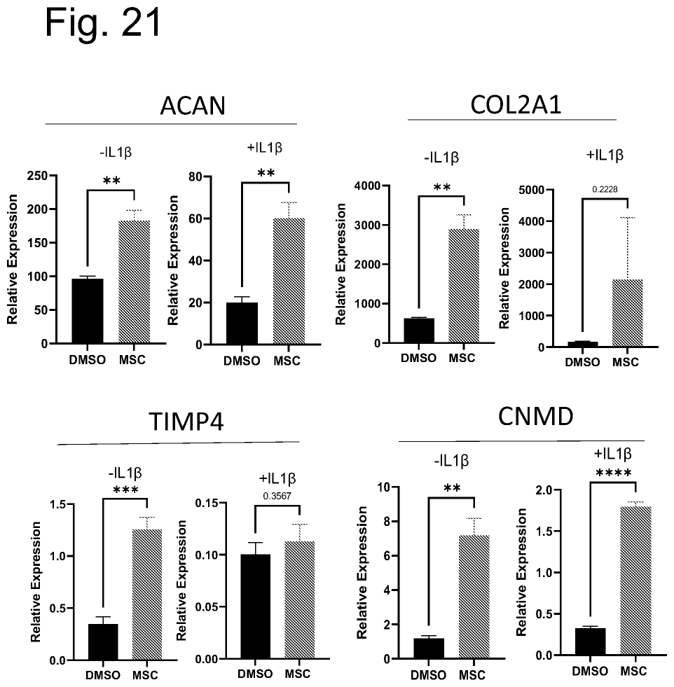

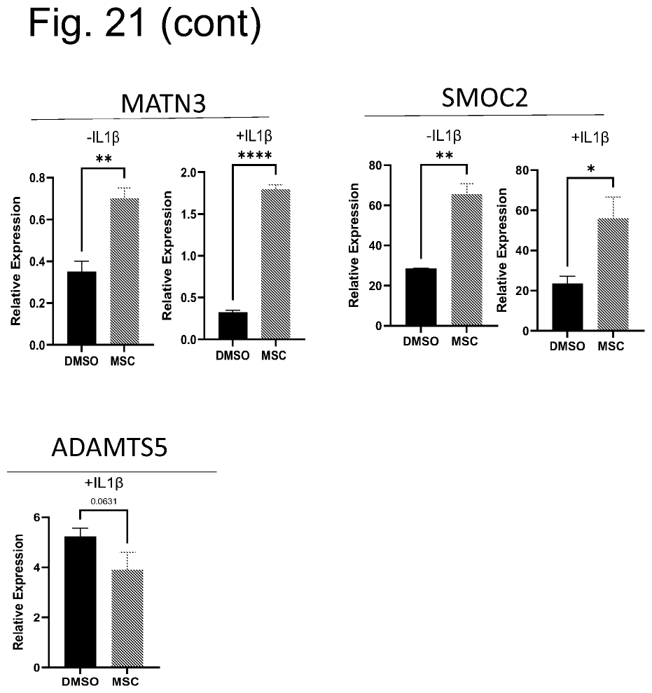

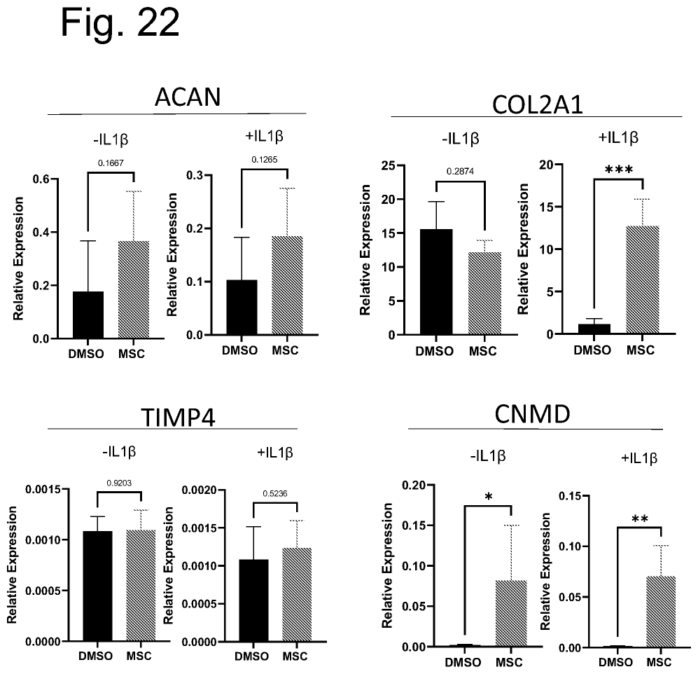

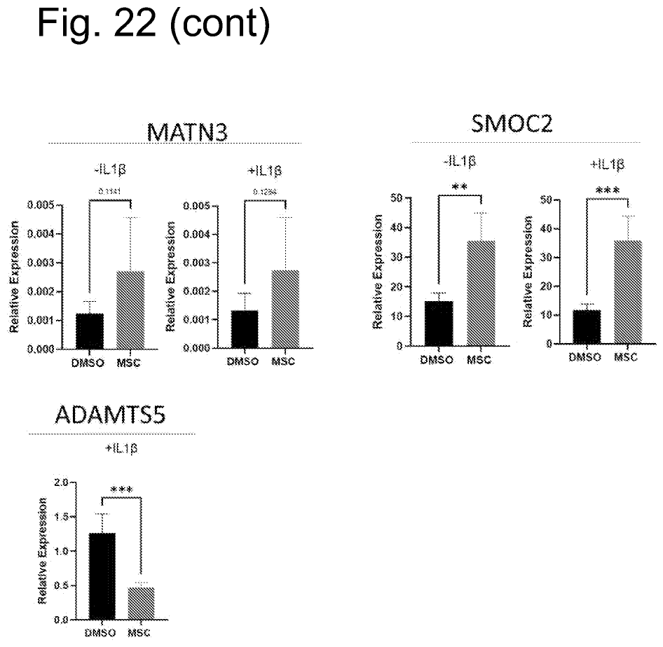

To extend these data immortalized human mesenchymal stromal cells (ihMSCs) were treated with MSC2530818 during chondrogenesis and in an IL1β OA disease degradation model. An increase in glycosaminoglycan (GAG) deposition was apparent with MSC2530818 treatment compared to DMSO control in both conditions, indicating the pro anabolic effect of CDK8i. Gene expression analysis of ihMSC micromasses treated with 100 nM MSC2530818 induced and a significant increase in healthy matrix component gene expression in chondrogenic (−IL1β) and OA disease model (+IL1β) conditions; i.e. ACAN (3-fold, 1.6-fold respectively) and MATN3 (4-fold, 3-fold respectively), and a 1.3-fold reduction gene expression of the aggrecanase ADAMTS5 (FIG. 19). The effect of MSC2530818 was similar in primary chondrocytes from human, canine and equine articular chondrocytes (with and without IL1β treatment). In general, an anabolic and anti-catabolic response is observed across species and in an IL-1β driven OA model (see FIGS. 20-22).

Modulation of Chondrocyte Hypertrophy Observed with BI1347 Treatment is a Class Effect of CDK8 Inhibition

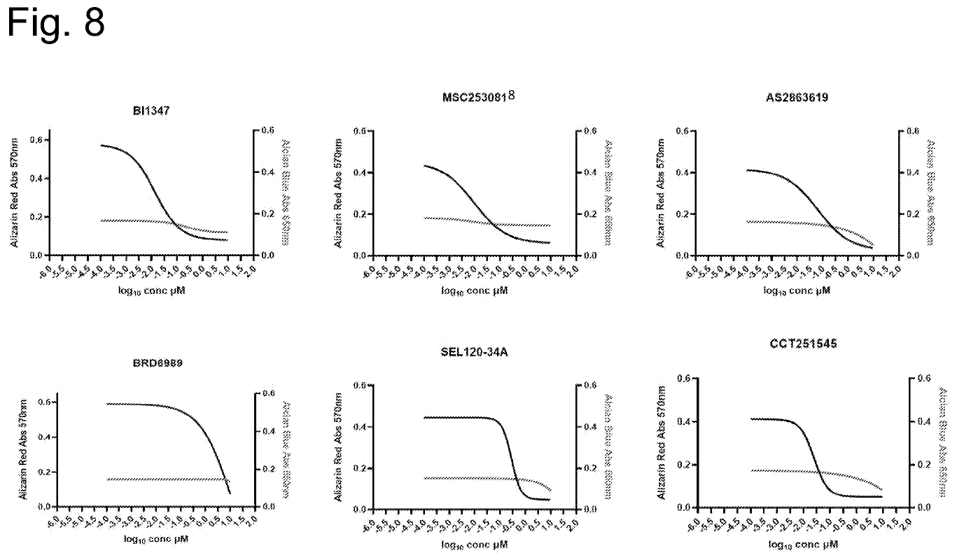

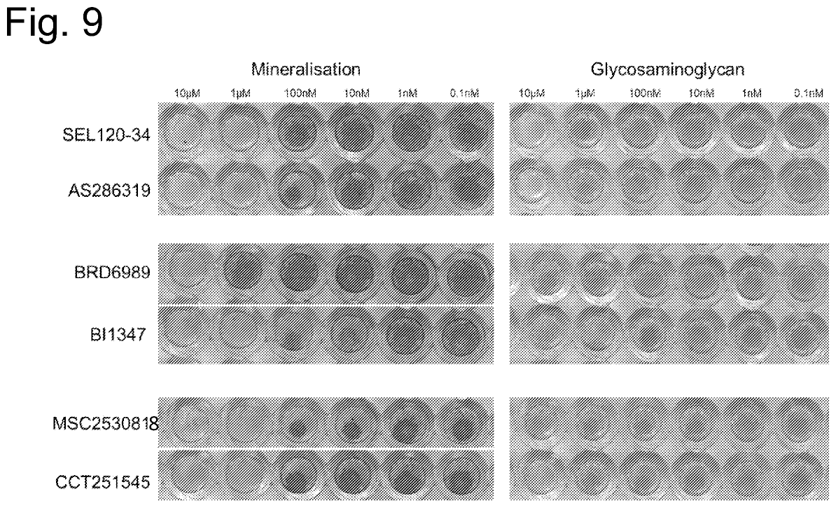

To determine whether the effects of BI1347 on endochondral ossification was specific to BI1347 or a class effect of CDK8 inhibition, several compounds from the drug class of CDK8 inhibitors were selected for a dose response assay on miniaturised ATDC5 micromasses. As shown in FIG. 8, similar trends are observed across the compounds with a distinct decrease in mineral deposition shown by alizarin red staining, and maintenance in GAG deposition shown by alcian blue staining. For compounds AS286319, SEL120-34A and CCT251545 alcian blue staining is modestly reduced at the highest concentration of 10 μM, however for BI1347, MSC2530818 and BRD6989 alcian blue is maintained at all concentrations. Interestingly, micromasses all have a similar appearance when treated with CDK8 inhibitors (FIG. 9). Changes in GAG deposition were not seen when BI1347 and MSC2530818 was provided to a monolayer culture of ATDC5 cells, emphasising the importance of using a micromass for screening.

### CDK8 Inhibition Via B11347 Treatment Displays Anti-Osteoclastic Activity In Vitro

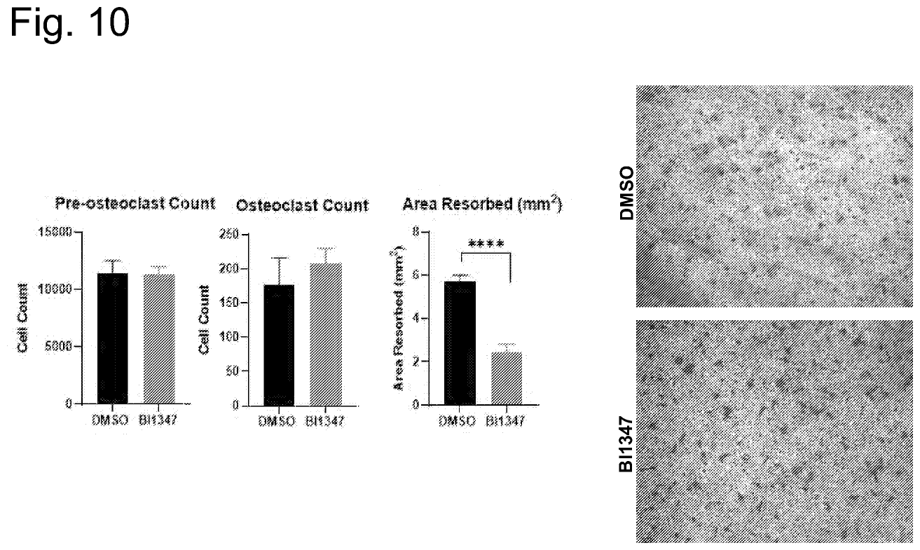

Treating C57/BL-6 murine bone marrow derived cells and differentiating them into osteoclasts in the presence of 1 μM BI1347 showed no effect on pre-osteoclast or mature osteoclast numbers compared to DMSO control (N=8) (FIG. 10). However, CDK8 inhibition via BI1347 treatment significantly reduces osteoclast resorption by 2.3-fold (p≤0.0001), suggesting CDK8 may play a role in osteoclastic activity. This is particularly important as increased osteoclast resorptive activity has been found to correlate with disease progression in OA and degenerative disc disease (Löfvall, Newbould et al. 2018, Zhu, Zhang et al. 2021).

### CDK8 Inhibition via B11347 Treatment Inhibits Mineralisation of MC3T3 Osteoblast-Like Cells and is not Due to Toxicity

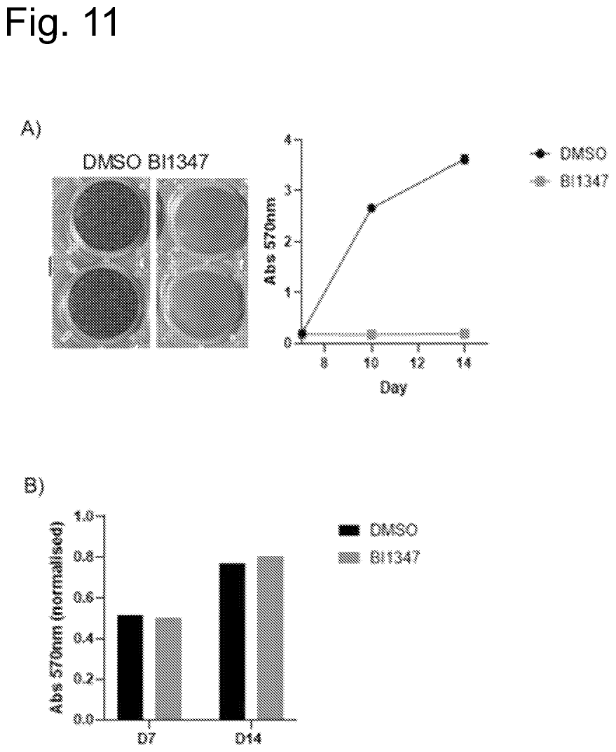

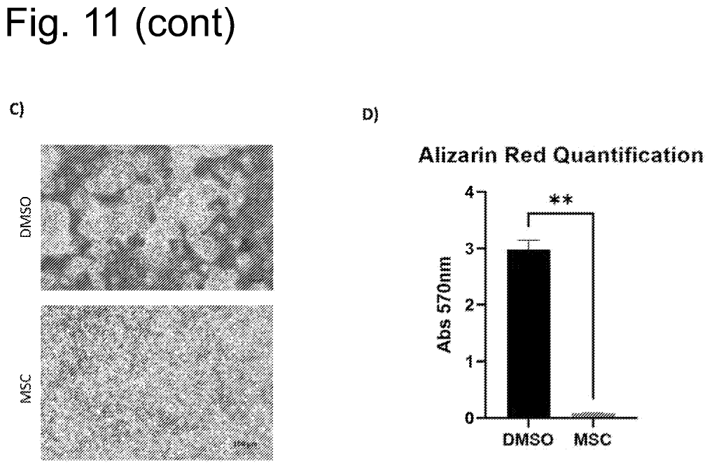

Treating MC3T3 osteoblast-like cells (subclone 14) with 1 μM BI1347 throughout differentiation completely inhibited mineralisation as no alizarin red staining is observed (FIG. 11). This contrasts with DMSO control, where a progressive increase in mineralisation is observed over the 14 day culture period. To ensure that this ablation of mineralisation in the BI1347 treated cells was not due to toxicity from the compound, a cell viability assay was carried out on day 7 and 14 of culture, with no difference in absorbance observed between DMSO control and 1 μM BI1347 treated cells. This indicates that BI1347 inhibits mineralisation of MC3T3 cells and may have an inhibitory role in osteoblast differentiation. In a further optimised MC3T3 (subclone 14) osteogenic assay, where MSC2530818 was present at 100 nM throughout the culture, as similar result was observed where mineralisation was ablated in comparison to DMSO treated cultures (FIG. 11). This may be of benefit to degenerative cartilage diseases as often pathological bone formation in the form of osteophytes is present. Furthermore, subchondral bone remodelling leading to sclerosis is mediated by osteoblast (and osteoclast) activity.

### CDK8 Inhibition Via BI1347 Treatment Promotes Anti-Inflammatory Gene Expression in THP-1 Derived Macrophages and Reduces IL1β Secretion

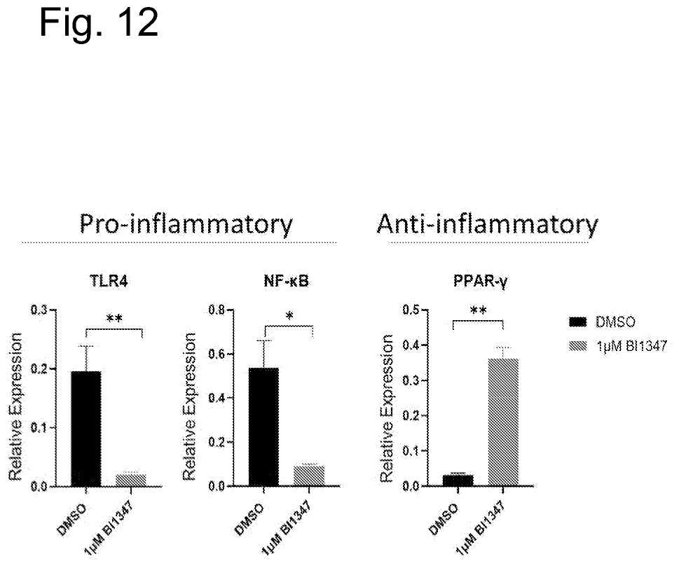

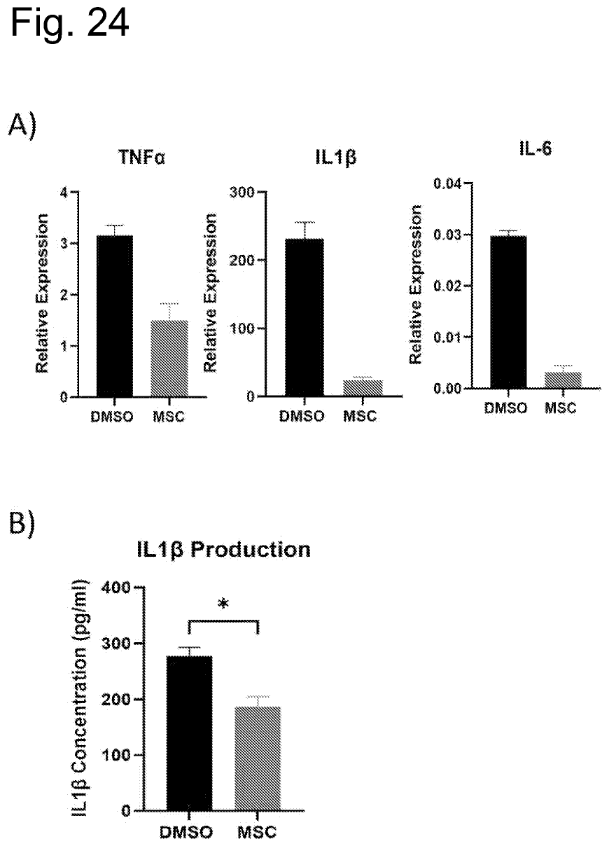

Treating THP-1 derived macrophages with 1 μM BI1347 1 hour prior and throughout the subsequent 24-hour LPS stimulation period, significantly reduces gene expression of pro-inflammatory pathway mediators TLR4 9-fold (p≤0.01) and NF-κB 6-fold (p≤0.05) whilst upregulating expression of anti-inflammatory pathway mediator PPAR-γ 9-fold (p≤0.01) (FIG. 12). Furthermore, MSC2530818 (100 nM) reduced expression of the pro-inflammatory cytokines TNFα (2.1-fold; p≤0.01), IL1β (9.8-fold; p≤0.01) and IL-6 (10-fold; p≤0.0001) from THP-1 derived macrophages compared to DMSO control (FIG. 24). Secretion of IL1β into the culture media from THP-1 derived macrophages was reduced by 1.5-fold (p≤0.05).

This is likely to be of benefit to degenerative cartilage diseases as inflammatory processes are thought to play a key role in progressive cartilage destruction. Inflammatory pathways promote the upregulation of cartilage degradation mediators, in addition to increasing associated joint pain. Importantly, PPAR-γ suppression is widely documented in OA and deemed an important therapeutic target due to its suppressive role in chondrocyte terminal differentiation.

RNA-Seq of BI1347 Treated Micromasses Suggests CDK8 inhibition promotes Anabolic Chondrogenesis and Reduces Hypertrophic and Inflammation Markers

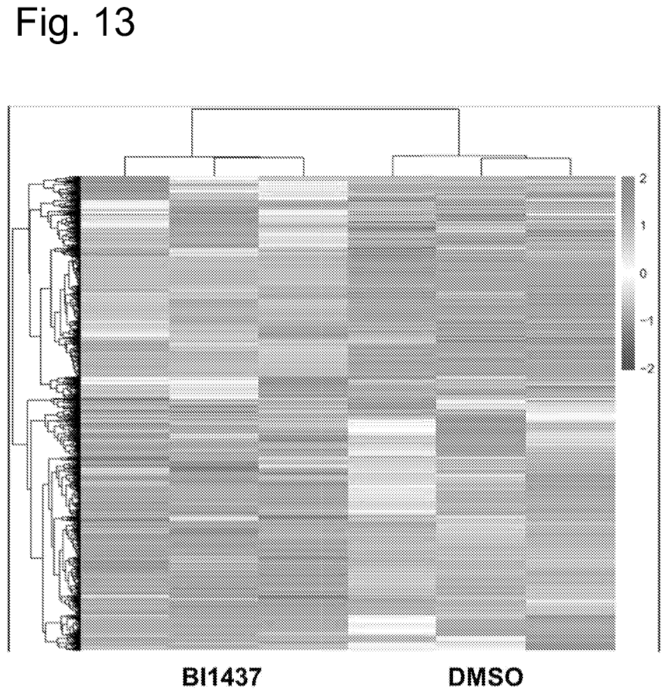

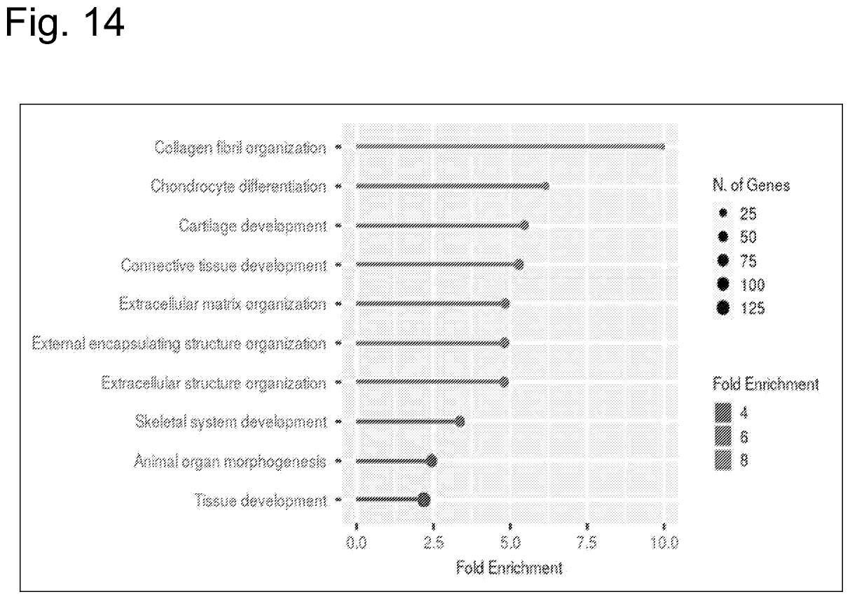

BI1347 treated ATDC5 micromasses on day 7 of differentiation display considerably different gene expression profiles compared to DMSO control suggesting CDK8 inhibition dramatically alters cell phenotype (FIG. 13). Upon analysis of the upregulated differentially expressed genes (DEGs), it is evident that CDK8 inhibition via BI1347 treatment promotes healthy chondrogenesis by significant enrichment of chondrogenic mediators (GDF5, FGFR3, SOX5,6,8 and CNMD) (Table 3 and FIGS. 14 and 16). This is supported by the upregulation of genes encoding key ECM components of healthy cartilage such as Collagen Type 2 and 9, Aggrecan, Matrilin 1 and 3. Furthermore, a number of genes encoding BMP inhibitors were found to be upregulated such as SMOC2, NOG and GREM1. Additionally, the second highest DEG upregulated was TIMP4 (Log₂ Fold increase of 6.85) which encodes an MMP inhibitor implicated in preventing the degradation of cartilage ECM. Interestingly, pathway analysis displayed notable enrichment of healthy ECM components in addition to processes involved in cartilage development.

TABLE 3
RNA-Seq Top 10 Upregulated Differentially Expressed Genes (DEGs.
Table of 10 most upregulated DEGs shows a significant increase in
known chondroprotective genes TIMP4 (Log₂ Fold 6.85) and
Chondromodulin-1 (Log₂ Fold 6.81).
Top 10 Upregulated Protein Encoding Genes

| Log₂ | Gene | |
|---|---|---|
| Fold Change | Name | Protein Encoded |
| 7.57 | CPA5 | Carboxypeptidase A5 |
| 6.85 | TIMP4 | Metalloproteinase Inhibitor 4 |
| 6.81 | CNMD | Chondromodulin |
| 6.76 | F5 | Coagulation Factor V |
| 6.76 | CPM | Carboxypeptidase M |
| 6.62 | PHLDA2 | Pleckstrin Homology-like Domain Family |
| | | A member 2 |
| 6.56 | GALNT17 | Polypeptide N-Acetylgalactosaminyl- |
| | | transferase 17 |
| 6.45 | KLK11 | Kallikrein Related Peptidase 11 |
| 6.42 | BARX2 | BARX Homeobox 2 |
| 6.40 | P2RY10 | Putative P2Y Purinoceptor 10 |

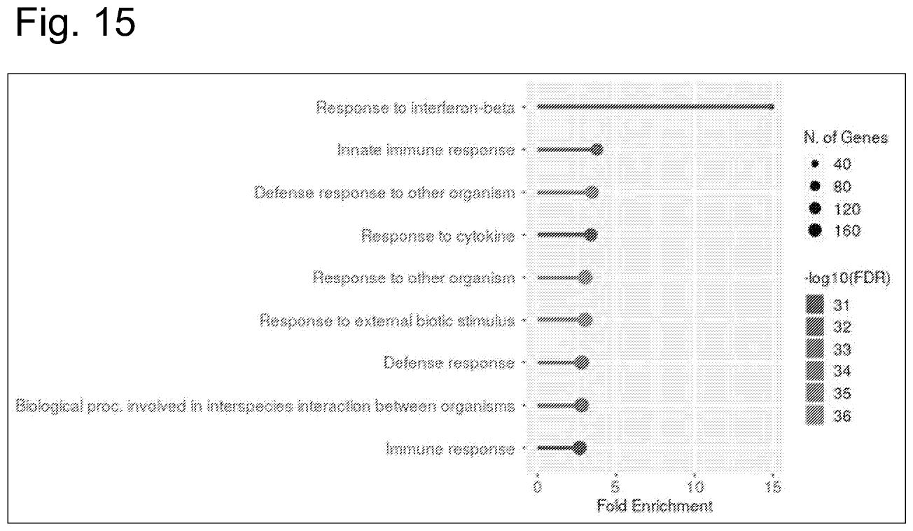

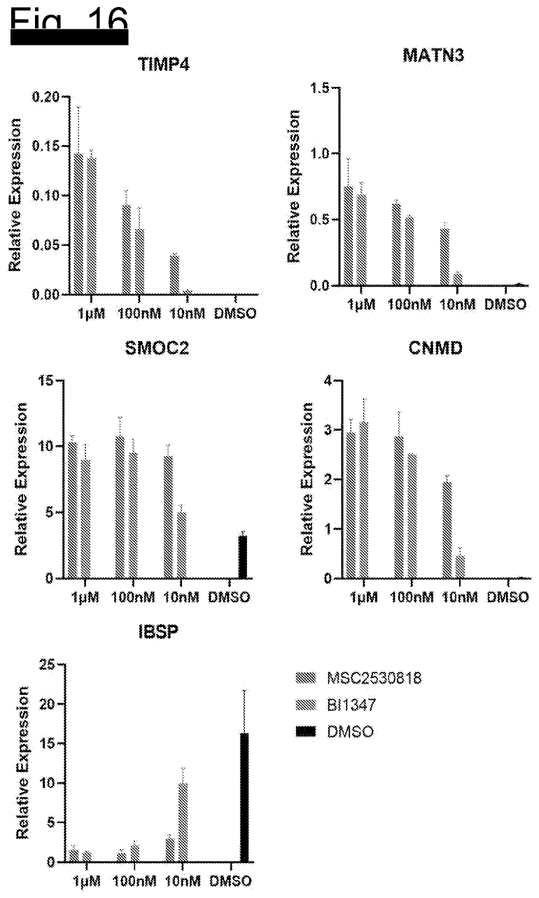

It is evident when looking at the downregulated DEGs that genes encoding proteins involved in inflammation, such as CXCL11, IL-6, ISG-15 and NOSTRIN are dramatically reduced in BI1347 treated cells compared to control (Tables 4 and 5, FIG. 15). In addition, mediators of chondrocyte hypertrophy (IHH, MEF2C, and TMEM119), matrix mineralisation (IBSP, ISG-15 and BGLAP) and canonical Wnt signalling (AXIN2 and ISG-15) are also found to be downregulated. Interestingly, key enzymes involved in cartilage destruction such as ADAMTS4, are significantly downregulated with BI1347 treatment.

TABLE 4
RNA-Seq Top 10 Downregulated Differentially Expressed Genes (DEGs).
Table of 10 most downregulated DEGS shows a significant decrease in
inflammatory mediators related to cartilage destruction such as
CXCL11 (Log₂ Fold −7.89).
Top 10 Downregulated Protein Encoding Genes

| | Gene | |
|---|---|---|
| log₂FoldChange | Name | Protein Encoded |
| −11.27 | OAS2 | 2′-5′-oligoadenylate Synthetase 2 |
| −10.46 | OASL1 | 2′-5′-oligoadenylate Synthetase-like 1 |
| −8.53 | SLC36A2 | Proton-coupled Amino Acid Transporter 2 |
| −7.89 | CXCL11 | C-X-C Motif Chemokine 11 |
| −7.66 | GRHL3 | Grainyhead Like Transcription Factor 3 |
| −7.64 | OAS1A | 2′-5′-oligoadenylate Synthetase 1 Alpha |
| −7.22 | OAS3 | 2′-5′-oligoadenylate Synthetase 3 |
| −7.12 | RSAD2 | Radical S-Adenosyl Methionine Domain |
| | | Containing 2 |
| −7.12 | TRIM30D | Tripartite Motif-containing 30D |
| −7.03 | MX2 | Interferon-induced GTP-binding Protein |
| | | MX2/MXB |

TABLE 5
RNA-Seq Selected DEGs of Interest. Tables of selected interesting DEGs in both the
upregulated (Top) and downregulated (Bottom) data sets including the name of the protein
encoded by the gene and documented function in relation to cartilage health and disease.
Selected Upregulated Genes of Interest

| Log₂ | | | |
|---|---|---|---|
| Fold | Gene | | |
| Change | Name | Protein Encoded | Function In Relation To Cartilage |
| 6.85 | TIMP4 | Metalloproteinase | Inhibitor of MMP-1, -2, -3, -7, -8, -9, -14, -26 and |
| | | Inhibitor 4 | ADAM-17, -28, ADAMT S-4 |
| 6.81 | CNMD | Chondromodulin-1 | Stabilizes chondrocyte phenotype by promoting |
| | | | chondrogenesis and inhibiting hypertrophic differentiation |
| 5.88 | MATN1 | Matrillin 1 | Adapter protein binding to collagen II and IX to form a |
| | | | filamentous network |
| 5.87 | MATN3 | Matrillin 2 | Adapter protein binding to collagen II and IX to form a |
| | | | filamentous network |
| 4.44 | COL9A3 | Collagen Type 9 | Essential component in healthy cartilage contributing |
| | | | to the stabilisation of the fibrillar collagen network |
| | | | in the cartilage matrix |
| 2.71 | SMOC3 | SPARC Related Modular | Modulator of BMP and Wnt Signalling |
| | | Calciium Binding 2 | |
| 2.69 | COL2A1 | Collagen Type 2 | The most abundant matrix component of healthy cartilage |
| | | | and is essential for structural cartilage integrity |
| 2.65 | SOX8 | SRY (sex determining | Co-expressed with SOX9 in articular chondrocytes and |
| | | region Y)-box 8 | contributes to articular cartilage integrity |
| 2.40 | ACAN | Aggrecan | Largest and most abundant proteoglycan in healthy |
| | | | articular cartilage |
| 2.39 | GDF5 | Growth Differentiation | An extracellular matrix signalling molecule involved |
| | | Factor 5 | in stabilising mature chondrocyte phenotype through |
| | | | Wnt modulation |
| 2.08 | NOG | Noggin | Inhibitor of chondrocyte hypertrophythrough BMP/Wnt |
| | | | signalling suppression |
| 1.75 | FGFR3 | Fibroblast Growth | Inhibits chondrocyte hypertrophythrough suppression |
| | | Factor Receptor 3 | of IHH |
| 1.68 | GREM1 | Gremlin 1 | BMP antagonist with negative effects on matrix |
| | | | mineralisation |
| 1.16 | SOX5 | SRY (sex determining | In combination with sox6 and 9, promotes anabolic |
| | | region Y)-box 5 | chondrogenesis |
| 1.11 | SOX6 | SRY (sex determining | In combination with sox5 and 9, promotes anabolic |
| | | region Y)-box 6 | chondrogenesis |
| −7.89 | CXCL11 | C-X-C motif | Stimulates release of inflammatory mediators, |
| | | chemokine 11 | MMPs and induction of chondrocyte apoptosis |
| −6.47 | ISG15 | Interferon Stimulated | Co-expressed with markers of chondrocyte hypertrophy |
| | | Gene 15 | and involved in the positive regulation of bone |
| | | | mineralisation through FRZD9/Wnt signalling |
| −6.06 | IBSP | Integrin binding | Significant component in mineralised tissue matrix |
| | | sialoprotein; Bone | binding lightly to hydroxyapatite |
| | | sialoprotein 2; | |
| −4.99 | COL 13A1 | Collagen Type 13 | Non-fibrillar collagen found to be increased in OA |
| −4.98 | TMEM119 | Transmembrane | Associated with fibrosis associated chondrocyte |
| | | Protein 119 | phenotype located in and around cartilage lesions |
| −3.23 | IL-6 | Interleukin 6 | Proinflammatory cytokine upregulating expression of |
| | | | inflammatory mediators, MMPs and ADAMT Ss |
| −2.65 | IHH | Indian Hedgehog | Inducer of chondrocyte hypertrophy |
| −2.38 | AXIN2 | Axis Inhibition | Transcriptional target of canonical Wnt signalling |
| | | Protein 2 | |
| −2.00 | NOSTRIN | Nitrix Oxide | Involved in nitric oxide (NO) production. NO induces |
| | | Synthasese | inflammation, cartilage destruction and chondrocyte |
| | | Trafficker | apoptosis |
| −1.58 | MEF2C | Myocyte-specific | Pro-hypertrophic transcription marker in chondrocytes |
| | | Enhancer Factor 2C | |
| −1.58 | ADAMT S4 | ADAM Metallopeptidase | Major aggrecanase involved in aggrecan degradation |
| | | with Thrombospondin | |
| | | Type 1 Motif 4 | |
| −1.42 | BGLAP | Osteocalcin | Mediator of matrix mineralisation |

In summary, it is evident that CDK8 inhibition via BI1347 treatment dramatically enhances chondrogenesis and seemingly stabilises chondrocytes by downregulation of hypertrophic mediators. Furthermore, healthy ECM components are enriched whilst matrix mineralisation mediators and catabolic enzymes are supressed, giving rise to the morphological changes observed in culturing chondrogenic micromasses with CDK8 inhibitors.

CDK8i Induces a Dose Dependant Modulation of Gene Biomarkers, with Short-Term Dosing Required for Long-Term Effect.

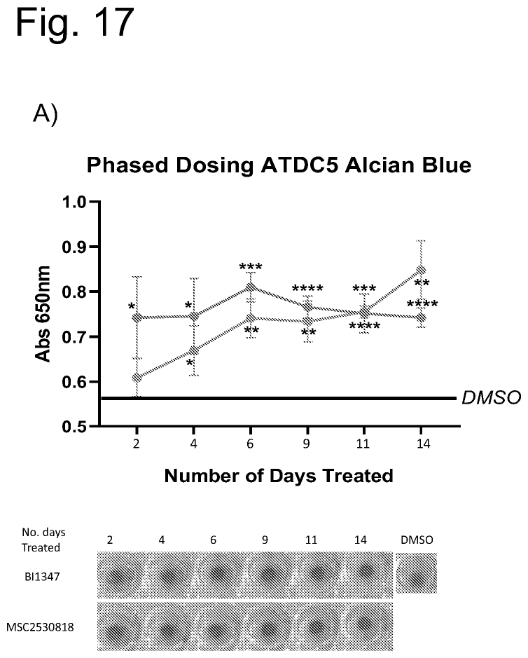

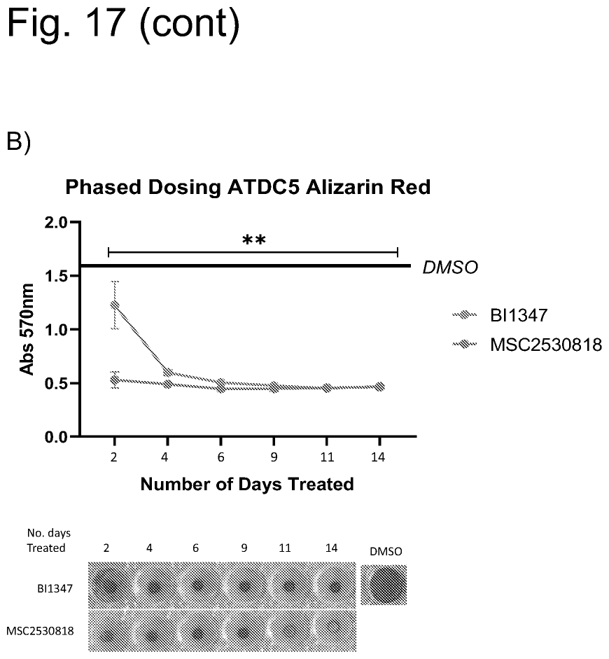

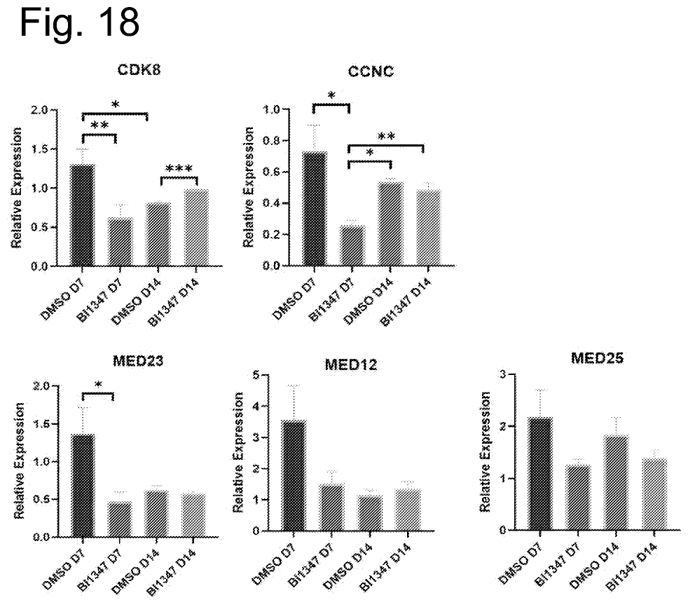

Using these genes as biomarker readouts, a dose response in gene expression was observed with efficacy down to 10 nM with both MSC2530818 and BI1347 (FIG. 16) following 14 days differentiation to hypertrophy. Furthermore, these molecules only require a short exposure time (<48 hrs) in culture to modify long term chondrocyte phenotype equivalent to that when molecules are present for the entire culture period (FIG. 17). Glycosaminoglycan (GAG) deposition increased with CDK8i compared to DMSO control at all dosing durations, conversely mineralization was decreased with CDK8i compared to DMSO at all dosing durations. Interestingly, Mediator complex gene expression were robustly downregulated at day 7 compared to day 14 in hypertrophically differentiated ATDC5s with BI1347 treatment (FIG. 18). This is consistent with the hypothesis that modulation of early mediator complex level through CDK8i has the capacity to alter cell transition towards hypertrophy.

### MSC2530818 Modifies Extracellular Acidification Rate of Hypertrophic ATDC5 Micromasses

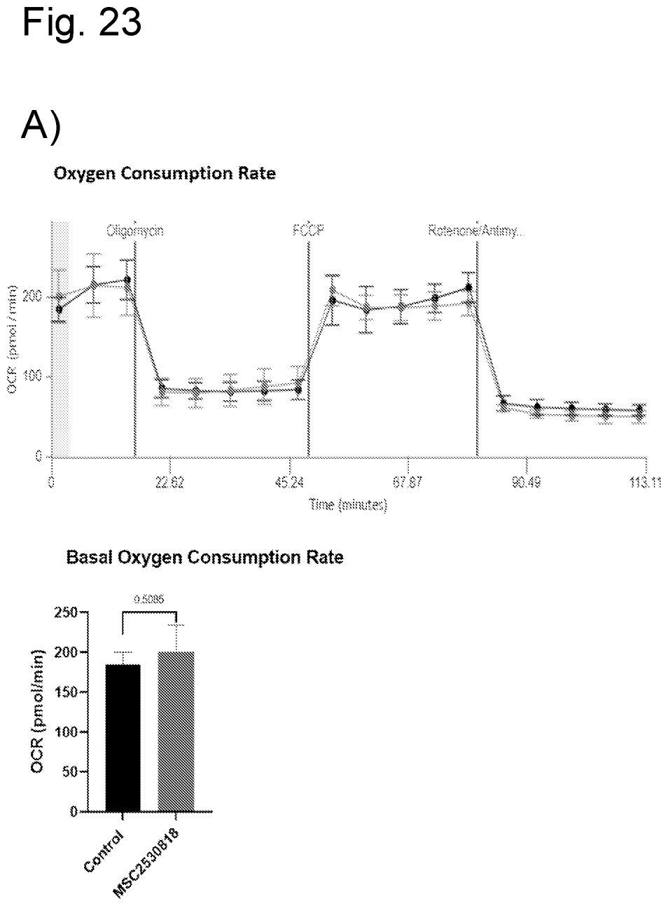

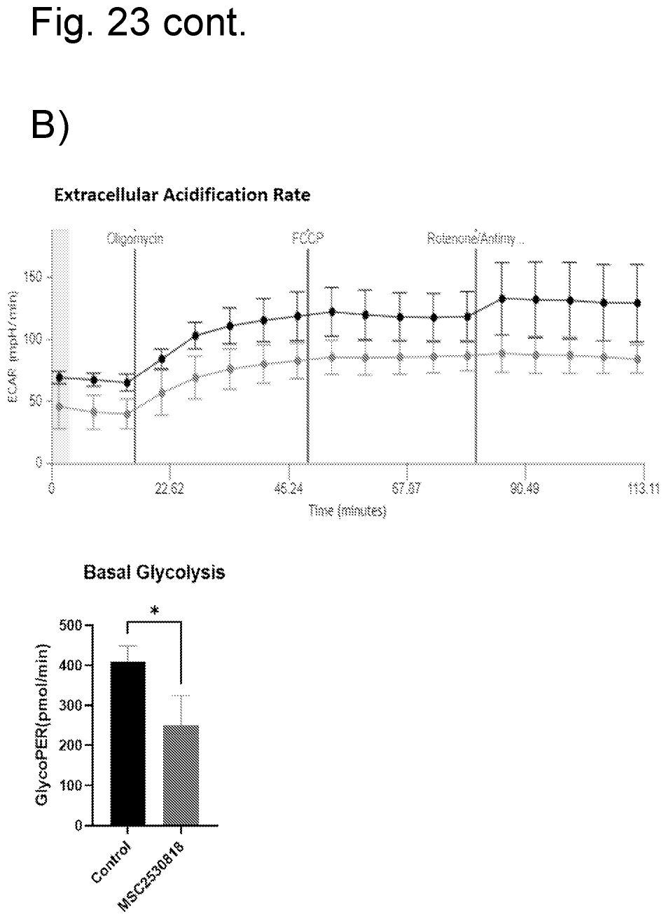

Basal metabolic analysis of MSC2530818 treated hypertrophic ATDC5 micromasses displayed no difference in of basal oxygen consumption rate compared to DMSO control (p=0.5085). Conversely, extracellular acidification rate from MSC2530818 treated micromasses was lower than DMSO control across the time course measured. A 37% decrease (p<0.05) in total basal glycolysis (inferred from OCR/ECAR measurements) was observed with MSC2530818 treatment compared to DMSO control (FIG. 23). Cell metabolism is modulated OA with increased reliance in glycolysis observed in disease. It has been postulated that metabolism alone is a potential disease modifying target for future OA drug development.

### In Vivo Analysis

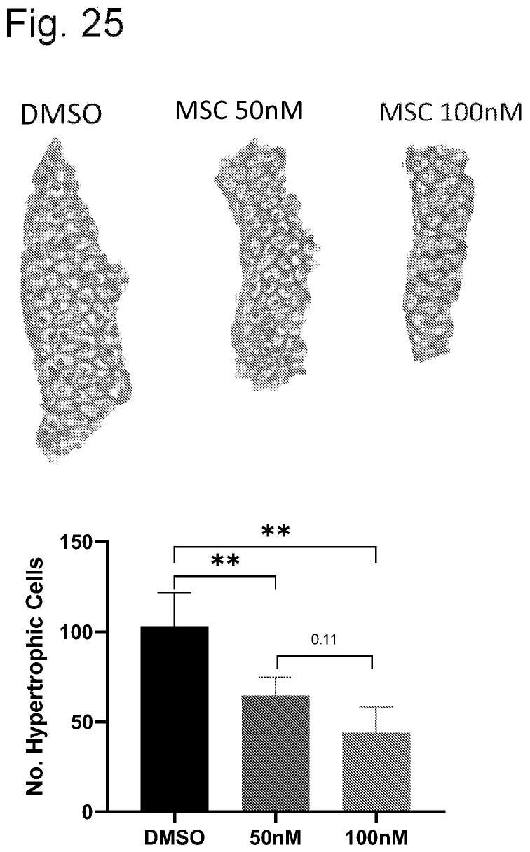

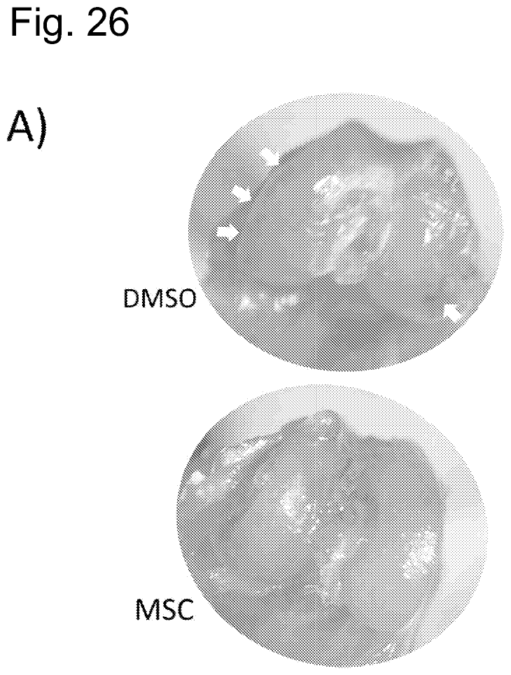

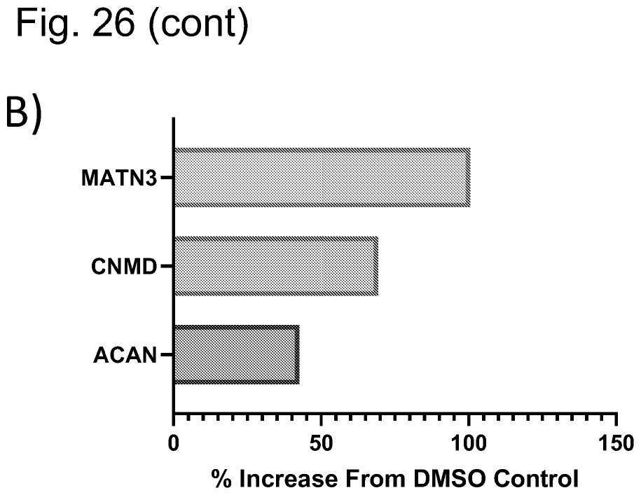

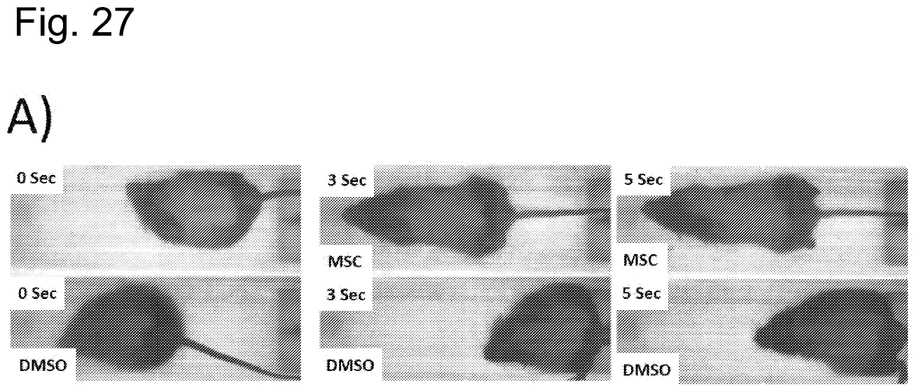

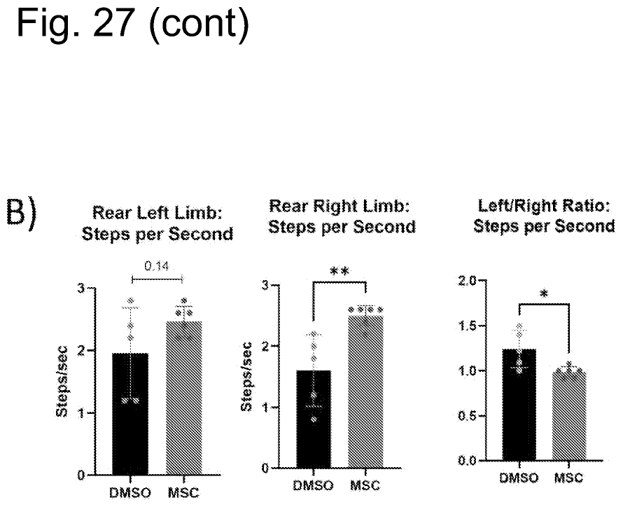

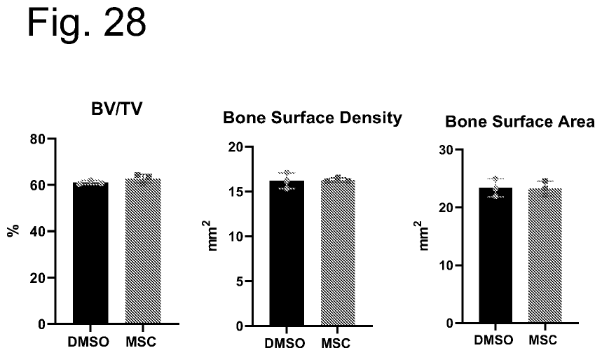

Increasing concentrations of MSC2530818 caused a reduction in enlarged/hypertrophic cells in 5 dpf zebrafish embryos at sites of future ossification in the zebrafish jaw (FIG. 25). Additionally, our biomarker study revealed that blood vessels could be identified in the periphery of the articular cartilage in STR/Ort mice treated with DMSO (in region analogous to osteophyte formation), but absent in mice treated with 5 mg/kg MSC2530818 for 2 weeks (FIG. 26). Furthermore, increased cartilage gene expression associated with anabolic action of chondrocytes was observed with MSC2530818 treatment compared to DMSO (FIG. 26). Critically, MSC2530818 modified step symmetry in STR/Ort mouse model of spontaneous OA, which can be used as a surrogate for correction of limping (FIG. 27). Finally, upon analysis of subchondral bone by micro-computer tomography (FIG. 28) we did not observe an alteration in bone volume fraction (bone volume/total volume; BV/TV), Bone Surface Density or Bone Surface Area, indicating that MSC2530818 does not have an anabolic or catabolic effect on bone.

1. A method of treating a disorder characterised by chondrocyte hypertrophy in an individual, comprising administering a Mediator kinase inhibitor to the individual.

2. A Mediator kinase inhibitor for use in a method of treating a disorder characterised by chondrocyte hypertrophy in an individual, the method comprising administering the Mediator kinase inhibitor to the individual.

3. The method of claim 1, or the Mediator kinase inhibitor for use of claim 2, wherein the Mediator kinase inhibitor is a cyclin-dependent kinase 8 (CDK8) inhibitor and/or a cyclin-dependent kinase 19 (CDK19)

4. The method of claim 1 or 3, or the Mediator kinase inhibitor for use of claim 2 or 3, wherein the Mediator kinase inhibitor
(a) increases expression and/or deposition of a cartilage extracellular matrix component, optionally wherein the Mediator kinase inhibitor increases expression of an anabolic mediator of cartilage extracellular matrix;
(b) increases expression of (i) an inhibitor of a matrix degrading enzyme, and/or (ii) an inhibitor of angiogenesis/hypertrophy;
(c) reduces mineralization of cartilage extracellular matrix, optionally wherein the Mediator kinase inhibitor decreases expression of a promoter of chondrocyte hypertrophy or promoter of matrix mineralization or increases expression of an inhibitor of chondrocyte hypertrophy or matrix mineralisation:
(d) inhibits (i) osteoblast mineralisation and/or (ii) osteoblast differentiation;
(e) reduces osteoclast resorption;
(f) has an anti-inflammatory effect, optionally wherein the Mediator kinase inhibitor (i) reduces expression of a pro-inflammatory mediator and/or (ii) increases expression of an anti-inflammatory mediator; and/or
(g) reduces chondrocyte glycolysis.

5. The method of claims 1, 3 and 4, or the Mediator kinase inhibitor for use of claim 2, 3 or 4, wherein the Mediator kinase inhibitor comprises:
(a) BI1347, BRD6989, AS2863619, SEL120-34A, MSC2530818 or CCT251545; or
(b) BI1347;
(c) MSC2530818.

6. The method of any one of claims 1 and 3 to 5, or the Mediator kinase inhibitor of any one of claims 2 to 5, wherein the individual is a mammal, optionally a human, dog, cat or horse.

7. A method of producing a miniaturised model of endochondral ossification, comprising:
(a) providing a micromass of ATDC5 cells having a volume of about 1 μl to about 7 μl;
(b) culturing the micromass in differentiation medium for at least 3 days; and
(c) culturing the micromass in mineralisation medium for a further 2 days or more.

8. A miniaturised model of endochondral ossification, producible by the method of claim 7.

9. A method of screening for compositions for use in treating (1) a disorder characterised by chondrocyte hypertrophy or (2) a fracture, comprising:
(a) providing a micromass of ATDC5 cells having a volume of about 1 μl to about 7 μl;
(b) culturing the micromass in differentiation medium for at least 3 days, wherein a test composition is provided to the micromass on day 1 or day 2;
(c) culturing the micromass in mineralisation medium comprising the test composition for a further 2 days or more; and
(d) quantifying the amount of proteoglycans and/or glycosaminoglycans, and/or the degree of mineralisation, in the micromass following the culture of step (c).

10. The method of claim 9, wherein (1) an increase or stabilization in the quantified amount of proteoglycans and/or glycosaminoglycans, and/or a decrease in the quantified degree of mineralisation, indicates that the test composition has utility in treating the disorder characterised by chondrocyte hypertrophy, and/or (2) a decrease or stablization in the quantified amount of proteoglycans and/or glycosaminoglycans, and/or an increase in the quantified degree of mineralisation, indicates that the test composition has utility in treating fracture.

11. The method of claim 10, wherein:
(i) the increase in the quantified amount of proteoglycans and/or glycosaminoglycans is relative to the quantified amount of proteoglycans and/or glycosaminoglycans respectively for a negative control composition; and/or
(ii) the decrease in the quantified degree of mineralization is relative to the quantified degree of mineralization for a negative control composition.

12. The method of any one of claims 9 to 11, wherein the amount of proteoglycans and/or glycosaminoglycans is quantified by alcian blue staining, and/or the degree of mineralization is quantified by alizarin red staining.

13. The method of any one of claims 9 to 12, wherein each of steps (a) to (d) are conducted in a single well of a microwell plate, optionally a plate comprising 96 microwells.

14. The method of claim 13, wherein steps (a) to (d) are repeated in parallel, wherein (i) each repeat is conducted in a different single well of the same microwell plate and (ii) a different test composition is provided to the micromass in each repeat.

15. The method of any one of claims 9 to 14, wherein steps (a) to (c) are performed in duplicate, and the amount of proteoglycans and/or glycosaminoglycans is quantified in one replicate and the degree of mineralization is quantified in another replicate.

16. The method any one of claims 7 and 9 to 15, wherein the micromass has a volume of about 1 μl to about 7 μl, optionally about 1 μl to about 5 μl, about 1.5 μl to about 3 μl, or about 2 μl.

17. The method any one of claims 7 and 9 to 16, wherein the micromass comprises about 5.0×10⁴ to about 6.0×10⁴ ATDC5 cells, optionally about 5.4×10⁴ ATDC5 cells.

18. The method any one of claims 7 and 9 to 17, wherein:
(a) the differentiation medium comprises DMEM-F12 supplemented with 5% FBS, 1% Ab/Am, 5 μg/ml human transferrin and 1×ITS; and/or
(b) the mineralisation medium comprises Alpha-MEM is supplemented with 5% FBS, 1% Ab/Am, 5 μg/ml human transferrin, 1×ITS, 7 mM β-glycerophosphate, and 50 μg/ml Ascorbic acid.

19. The method any one of claims 7 and 9 to 18, wherein culturing in step (b) and step (c) is performed in a volume of about 50 μl medium to about 400 μl medium, optionally about 100 μl medium.

20. A composition for use in treating (1) a disorder characterised by chondrocyte hypertrophy or (2) a fracture, identified by the method of any one of claims 9 to 19.

21. The method of any one of claims 1, 3 to 6 and 9 to 15, or the Mediator kinase inhibitor for use of any one of claims 2 to 6, wherein the disorder comprises loss and/or mineralisation of cartilage, optionally wherein the cartilage is articular cartilage.

22. The method or Mediator kinase inhibitor for use of claim 21, wherein the disorder is osteoarthritis or degenerative disc disease.

23. The method any one of claims 1, 3 to 6, 9 to 15 and 21, or the Mediator kinase inhibitor for use of any one of claims 2 to 6 and 21, wherein the disorder comprises abnormal formation of bone, optionally in soft tissue.

24. The method or the Mediator kinase inhibitor for use of claim 23, wherein the disorder is heterotopic ossification.

25. The method any one of claims 1, 3 to 6, 9 to 15, 21 and 23, or the Mediator kinase inhibitor for use of any one of claims 2 to 6, 21 and 23, wherein the disorder comprises formation of an osteocartilaginous mass, optionally wherein the disorder is hereditary multiple exostoses.


SEQUENCE LISTING (100 sequences)

SEQ ID NO: 1 - DNA, 20 bp, synthetic construct
  source 1..20: mol_type = other DNA
ggcaaaacaatgcaaacctt

SEQ ID NO: 2 - DNA, 21 bp, synthetic construct
  source 1..21: mol_type = other DNA
caagggcatatcctacgacaa

SEQ ID NO: 3 - DNA, 20 bp, synthetic construct
  source 1..20: mol_type = other DNA
gaggagatggagggtgaggt

SEQ ID NO: 4 - DNA, 20 bp, synthetic construct
  source 1..20: mol_type = other DNA
gatggtgatgtcctcctcgc

SEQ ID NO: 5 - DNA, 20 bp, synthetic construct
  source 1..20: mol_type = other DNA
ctggagactgctgaacgaga

SEQ ID NO: 6 - DNA, 20 bp, synthetic construct
  source 1..20: mol_type = other DNA
gagatgtgtgtctgctccgt

SEQ ID NO: 7 - DNA, 20 bp, synthetic construct
  source 1..20: mol_type = other DNA
tcctggtgtcaaaggtcaca

SEQ ID NO: 8 - DNA, 20 bp, synthetic construct
  source 1..20: mol_type = other DNA
tcccttagcaccatccagac

SEQ ID NO: 9 - DNA, 19 bp, synthetic construct
  source 1..19: mol_type = other DNA
ataggctcagtcggggctc

SEQ ID NO: 10 - DNA, 16 bp, synthetic construct
  source 1..16: mol_type = other DNA
ccggattgcaagcgcc

SEQ ID NO: 11 - DNA, 20 bp, synthetic construct
  source 1..20: mol_type = other DNA
ggggagcgacaatcacattt

SEQ ID NO: 12 - DNA, 20 bp, synthetic construct
  source 1..20: mol_type = other DNA
tctctcctccagcaaaacgg

SEQ ID NO: 13 - DNA, 20 bp, synthetic construct
  source 1..20: mol_type = other DNA
ttggtacacacggctgtcaa

SEQ ID NO: 14 - DNA, 20 bp, synthetic construct
  source 1..20: mol_type = other DNA
cgcacctgttacgaactgaa

SEQ ID NO: 15 - DNA, 20 bp, synthetic construct
  source 1..20: mol_type = other DNA
gcacctacagtcaggtccag

SEQ ID NO: 16 - DNA, 22 bp, synthetic construct
  source 1..22: mol_type = other DNA
aggcatcatctgtttttcctgt

SEQ ID NO: 17 - DNA, 21 bp, synthetic construct
  source 1..21: mol_type = other DNA
tggctcacgaaattggacatc

SEQ ID NO: 18 - DNA, 26 bp, synthetic construct
  source 1..26: mol_type = other DNA
aggtctagcaaacagttaccatgacc

SEQ ID NO: 19 - DNA, 20 bp, synthetic construct
  source 1..20: mol_type = other DNA
cagaccagcagcactccata

SEQ ID NO: 20 - DNA, 20 bp, synthetic construct
  source 1..20: mol_type = other DNA
gcagcattctggaaggagac

SEQ ID NO: 21 - DNA, 22 bp, synthetic construct
  source 1..22: mol_type = other DNA
ctggtgaaaaggacctctcgaa

SEQ ID NO: 22 - DNA, 28 bp, synthetic construct
  source 1..28: mol_type = other DNA
ctgaagtactcattatagtcaagggcat

SEQ ID NO: 23 - DNA, 24 bp, synthetic construct
  source 1..24: mol_type = other DNA
gaagaacaatccagactagcagca

SEQ ID NO: 24 - DNA, 25 bp, synthetic construct
  source 1..25: mol_type = other DNA
ccttatagggaacttcacatcacag

SEQ ID NO: 25 - DNA, 22 bp, synthetic construct
  source 1..22: mol_type = other DNA
cgcgtctccttcgagctgtttg

SEQ ID NO: 26 - DNA, 24 bp, synthetic construct
  source 1..24: mol_type = other DNA
tgtaaagtcaccaccctggcacat

SEQ ID NO: 27 - DNA, 22 bp, synthetic construct
  source 1..22: mol_type = other DNA
gctgcagtgatctcagaagaag

SEQ ID NO: 28 - DNA, 20 bp, synthetic construct
  source 1..20: mol_type = other DNA
gatggtgagggaagacccta

SEQ ID NO: 29 - DNA, 22 bp, synthetic construct
  source 1..22: mol_type = other DNA
gctggaagtcggagagccgaga

SEQ ID NO: 30 - DNA, 21 bp, synthetic construct
  source 1..21: mol_type = other DNA
agagaacgaaaccggggccac

SEQ ID NO: 31 - DNA, 20 bp, synthetic construct
  source 1..20: mol_type = other DNA
ctgccagtggaaaattaggg

SEQ ID NO: 32 - DNA, 20 bp, synthetic construct
  source 1..20: mol_type = other DNA
ttctcccttgtcaccacgat

SEQ ID NO: 33 - DNA, 20 bp, synthetic construct
  source 1..20: mol_type = other DNA
cctggttcatgggatgtttt

SEQ ID NO: 34 - DNA, 20 bp, synthetic construct
  source 1..20: mol_type = other DNA
caggaatgccttgttctcct

SEQ ID NO: 35 - DNA, 20 bp, synthetic construct
  source 1..20: mol_type = other DNA
tctcccgcatcatcgacact

SEQ ID NO: 36 - DNA, 21 bp, synthetic construct
  source 1..21: mol_type = other DNA
gtcggaataggtgttgagctg

SEQ ID NO: 37 - DNA, 21 bp, synthetic construct
  source 1..21: mol_type = other DNA
cctgaggacgttgagttttgc

SEQ ID NO: 38 - DNA, 20 bp, synthetic construct
  source 1..20: mol_type = other DNA
cagctcctaccttgagcagc

SEQ ID NO: 39 - DNA, 21 bp, synthetic construct
  source 1..21: mol_type = other DNA
cactcggctctagtgatacgg

SEQ ID NO: 40 - DNA, 21 bp, synthetic construct
  source 1..21: mol_type = other DNA
cttggccttctcgaacccttt

SEQ ID NO: 41 - DNA, 22 bp, synthetic construct
  source 1..22: mol_type = other DNA
aagatattgcctcacgctaccc

SEQ ID NO: 42 - DNA, 21 bp, synthetic construct
  source 1..21: mol_type = other DNA
tcctcaagagctgactgatgc

SEQ ID NO: 43 - DNA, 20 bp, synthetic construct
  source 1..20: mol_type = other DNA
ggagcgaggccatttacaac

SEQ ID NO: 44 - DNA, 22 bp, synthetic construct
  source 1..22: mol_type = other DNA
cgtagacaaggtagcccacttt

SEQ ID NO: 45 - DNA, 20 bp, synthetic construct
  source 1..20: mol_type = other DNA
atggagacggcgatagttcc

SEQ ID NO: 46 - DNA, 21 bp, synthetic construct
  source 1..21: mol_type = other DNA
ctagctgttacacccgagagt

SEQ ID NO: 47 - DNA, 19 bp, synthetic construct
  source 1..19: mol_type = other DNA
cgggtcgaggacctgtttg

SEQ ID NO: 48 - DNA, 23 bp, synthetic construct
  source 1..23: mol_type = other DNA
tgccgacatagaaattccagttc

SEQ ID NO: 49 - DNA, 23 bp, synthetic construct
  source 1..23: mol_type = other DNA
gtgaatgatacctacaggacgga

SEQ ID NO: 50 - DNA, 21 bp, synthetic construct
  source 1..21: mol_type = other DNA
gcaaaccactgtctagcatct

SEQ ID NO: 51 - DNA, 20 bp, synthetic construct
  source 1..20: mol_type = other DNA
ttgagccaacagcctttctt

SEQ ID NO: 52 - DNA, 20 bp, synthetic construct
  source 1..20: mol_type = other DNA
tcatgcattagctgctttgg

SEQ ID NO: 53 - DNA, 19 bp, synthetic construct
  source 1..19: mol_type = other DNA
attacaagggtgttcgaga

SEQ ID NO: 54 - DNA, 22 bp, synthetic construct
  source 1..22: mol_type = other DNA
tataacctctcttgctgccaga

SEQ ID NO: 55 - DNA, 21 bp, synthetic construct
  source 1..21: mol_type = other DNA
ctttgggggagactatggtgg

SEQ ID NO: 56 - DNA, 20 bp, synthetic construct
  source 1..20: mol_type = other DNA
tcgagccaggtgacaaactc

SEQ ID NO: 57 - DNA, 21 bp, synthetic construct
  source 1..21: mol_type = other DNA
tccggattctcaccaatcgaa

SEQ ID NO: 58 - DNA, 23 bp, synthetic construct
  source 1..23: mol_type = other DNA
cgatttccagcatcatctgtctc

SEQ ID NO: 59 - DNA, 20 bp, synthetic construct
  source 1..20: mol_type = other DNA
agcttgctggtgaaaaggac

SEQ ID NO: 60 - DNA, 20 bp, synthetic construct
  source 1..20: mol_type = other DNA
ttatagtcaagggcatatcc

SEQ ID NO: 61 - DNA, 20 bp, synthetic construct
  source 1..20: mol_type = other DNA
tccacagcctatccagaaca

SEQ ID NO: 62 - DNA, 20 bp, synthetic construct
  source 1..20: mol_type = other DNA
ctgctgctgttgtctctgct

SEQ ID NO: 63 - DNA, 17 bp, synthetic construct
  source 1..17: mol_type = other DNA
gcagcaagagcaaggac

SEQ ID NO: 64 - DNA, 17 bp, synthetic construct
  source 1..17: mol_type = other DNA
ttctgagagccctcggt

SEQ ID NO: 65 - DNA, 21 bp, synthetic construct
  source 1..21: mol_type = other DNA
ggacactccttgcaatttgag

SEQ ID NO: 66 - DNA, 21 bp, synthetic construct
  source 1..21: mol_type = other DNA
gtcattccactctcccttctc

SEQ ID NO: 67 - DNA, 18 bp, synthetic construct
  source 1..18: mol_type = other DNA
gatgggtgaggcgtgcag

SEQ ID NO: 68 - DNA, 20 bp, synthetic construct
  source 1..20: mol_type = other DNA
ggatctggactggcaggaac

SEQ ID NO: 69 - DNA, 20 bp, synthetic construct
  source 1..20: mol_type = other DNA
cggaggagcaaagtgctaca

SEQ ID NO: 70 - DNA, 20 bp, synthetic construct
  source 1..20: mol_type = other DNA
gttgtccttcacaggctggt

SEQ ID NO: 71 - DNA, 23 bp, synthetic construct
  source 1..23: mol_type = other DNA
ggcacaggaaaaacagatgatgc

SEQ ID NO: 72 - DNA, 20 bp, synthetic construct
  source 1..20: mol_type = other DNA
cttgttcggtccaaagcgtg

SEQ ID NO: 73 - DNA, 20 bp, synthetic construct
  source 1..20: mol_type = other DNA
actgtgaatgctacgagggc

SEQ ID NO: 74 - DNA, 20 bp, synthetic construct
  source 1..20: mol_type = other DNA
aacagtcgcagtgataggcg

SEQ ID NO: 75 - DNA, 20 bp, synthetic construct
  source 1..20: mol_type = other DNA
tgtctgaccaagaagctgcc

SEQ ID NO: 76 - DNA, 20 bp, synthetic construct
  source 1..20: mol_type = other DNA
gcaaactgtactcctccccc

SEQ ID NO: 77 - DNA, 20 bp, synthetic construct
  source 1..20: mol_type = other DNA
tgaggatttggaaagggtgt

SEQ ID NO: 78 - DNA, 20 bp, synthetic construct
  source 1..20: mol_type = other DNA
gagcacacagagggctacaa

SEQ ID NO: 79 - DNA, 20 bp, synthetic construct
  source 1..20: mol_type = other DNA
ccgccggctgtttaacttcg

SEQ ID NO: 80 - DNA, 20 bp, synthetic construct
  source 1..20: mol_type = other DNA
gcgctggaactcgtctcact

SEQ ID NO: 81 - DNA, 20 bp, synthetic construct
  source 1..20: mol_type = other DNA
actggcgagcactgtaacat

SEQ ID NO: 82 - DNA, 22 bp, synthetic construct
  source 1..22: mol_type = other DNA
agcatgtgaaagagtcgatggc

SEQ ID NO: 83 - DNA, 21 bp, synthetic construct
  source 1..21: mol_type = other DNA
tctcccggataatcgacactc

SEQ ID NO: 84 - DNA, 20 bp, synthetic construct
  source 1..20: mol_type = other DNA
caagggtgtgattcgaccca

SEQ ID NO: 85 - DNA, 20 bp, synthetic construct
  source 1..20: mol_type = other DNA
atggcagacgttgggaccat

SEQ ID NO: 86 - DNA, 21 bp, synthetic construct
  source 1..21: mol_type = other DNA
aatcgtcatgggagaggccaa

SEQ ID NO: 87 - DNA, 19 bp, synthetic construct
  source 1..19: mol_type = other DNA
atgacgacggcacctacag

SEQ ID NO: 88 - DNA, 21 bp, synthetic construct
  source 1..21: mol_type = other DNA
tcgcgttggggtaacttttca

SEQ ID NO: 89 - DNA, 18 bp, synthetic construct
  source 1..18: mol_type = other DNA
aggcggtgcttgttcctc

SEQ ID NO: 90 - DNA, 24 bp, synthetic construct
  source 1..24: mol_type = other DNA
gttcgagaagatgatctgactgcc

SEQ ID NO: 91 - DNA, 20 bp, synthetic construct
  source 1..20: mol_type = other DNA
agctacgaatctccgaccac

SEQ ID NO: 92 - DNA, 22 bp, synthetic construct
  source 1..22: mol_type = other DNA
cgttatcccatgtgtcgaagaa

SEQ ID NO: 93 - DNA, 23 bp, synthetic construct
  source 1..23: mol_type = other DNA
actcacctcttcagaacgaattg

SEQ ID NO: 94 - DNA, 23 bp, synthetic construct
  source 1..23: mol_type = other DNA
ccatctttggaaggttcaggttg

SEQ ID NO: 95 - DNA, 21 bp, synthetic construct
  source 1..21: mol_type = other DNA
atagcgagccacgcattcaca

SEQ ID NO: 96 - DNA, 22 bp, synthetic construct
  source 1..22: mol_type = other DNA
tgccagctcttctgtgtggttt

SEQ ID NO: 97 - DNA, 20 bp, synthetic construct
  source 1..20: mol_type = other DNA
aagaggaggtttcgccaccg

SEQ ID NO: 98 - DNA, 20 bp, synthetic construct
  source 1..20: mol_type = other DNA
aagggcaggggaagctgtca

SEQ ID NO: 99 - DNA, 21 bp, synthetic construct
  source 1..21: mol_type = other DNA
agcctgcatttctgcattctg

SEQ ID NO: 100 - DNA, 20 bp, synthetic construct
  source 1..20: mol_type = other DNA
tggcatctctgtgtcaacca